(19) United States
(12) Patent Application Publication
(10) Pub. No.: US 2025/0263705 A2
(43) Pub. Date: Aug. 21, 2025

(54) PHARMACEUTICAL COMBINATIONS FOR TREATMENT OF HBV

(71) Applicants: Souphalone LUANGSAY, Basel (CH); Henrik MUELLER, Basel (CH); Johanna Marie POSE VICENTE, Basel (CH); Malika AIT-GOUGHOULTE, Basel (CH); Wouter Hendrik Pieter DRIESSEN, Basel (CH)

(72) Inventors: Souphalone LUANGSAY, Basel (CH); Henrik MUELLER, Basel (CH); Johanna Marie POSE VICENTE, Basel (CH); Malika AIT-GOUGHOULTE, Basel (CH); Wouter Hendrik Pieter DRIESSEN, Basel (CH)

(73) Assignee: Hoffmann-La Roche Inc., Little Falls, NJ (US)

(21) Appl. No.: 18/659,831

(22) Filed: May 9, 2024

Related U.S. Application Data
(63) Continuation of application No. PCT/EP2022/081355, filed on Nov. 9, 2022.

(30) Foreign Application Priority Data
Nov. 11, 2021 (EP) 21207715.0

(51) Int. Cl. C12N 15/113; A61K 45/06; A61P 31/20

(52) U.S. Cl. CPC C12N 15/1131; A61K 45/06; A61P 31/20; C12N 15/1138; C12N 2310/11; C12N 2310/14; C12N 2310/3125; C12N 2310/315; C12N 2310/321; C12N 2310/322; C12N 2310/3341; C12N 2310/351

(57) ABSTRACT

The present invention is directed to pharmaceutical combinations for treating hepatitis B virus (HBV) infection comprising administering at least two, preferably two or three, different HBV therapeutics. In particular, the present invention relates to pharmaceutical combinations comprising an RNAi oligonucleotide targeting HBV and an anti-PDL1 antisense oligonucleotide.

### SEQUENCE LISTING

The instant application contains a Sequence Listing which has been submitted electronically in XML format and is hereby incorporated by reference in its entirety. Said XML copy, created on Aug. 16, 2024, is named 51551-019001_Sequence_Listing_8_16_24.xml and is 75,111 bytes in size.

### FIELD OF INVENTION

The present invention is directed to pharmaceutical combinations for treating hepatitis B virus (HBV) infection comprising administering at least two, preferably two or three, different HBV therapeutics. In particular, the present invention relates to pharmaceutical combinations comprising an RNAi oligonucleotide targeting HBV and an anti-PDL1 antisense oligonucleotide.

### BACKGROUND

HBV infection remains a major health problem worldwide which concerns an estimated 350 million chronic carriers. Approximately 25% of carriers can be predicted to die from chronic hepatitis, cirrhosis, or liver cancer. Hepatitis B virus is the second most significant carcinogen behind tobacco, causing from 60% to 80% of all primary liver cancer.

The outer envelope proteins of HBV are collectively known as hepatitis B surface antigen (HBsAg). HBsAg consists of three related polypeptides called S, M, and L encoded by overlapping open reading frames (ORF). The smallest envelope protein is S with 226 amino acids, called the S-ORF. M and L are produced from upstream translation initiation sites and add 55 and 108 amino acids, respectively, to S. HBV S, M, and L glycoproteins are found in the viral envelope of intact, infectious HBV virions, named Dane particles, and all three are produced and secreted in a vast excess that forms non-infectious subviral spherical and filamentous particles (both referred to as decoy particles) found in the blood of chronic HBV patients. The abundance of HBsAg on the surface of decoy particles is believed to inhibit humoral immunity and spontaneous clearance in patients with chronic HBV infection (CHB). The current standard of care for chronic HBV infection is treatment with oral nucleos(t)ide analogues such as entecavir or tenofovir which provide suppression of HBV replication by inhibiting HBV DNA synthesis but do not act directly on viral antigens, such as HBsAg. Nucleos(t)ide analogs, even with prolonged therapy, only show low levels of HBsAg clearance. In this respect, patients with chronic hepatitis B exhibit very weak HBV T-cell responses and lack anti-HBs antibodies, which is believed to be one of the reasons that these patients are not able to clear the virus.

A clinically important goal is to achieve a functional cure of chronic HBV infection, defined as HBsAg seroconversion and serum HBV-DNA elimination. This is expected to result in a durable response thereby preventing development of cirrhosis and liver cancer, and prolonging survival. Currently, chronic HBV infection cannot be eradicated completely due to the long term or permanent persistence of the viral genome as a covalently closed circular DNA (cccDNA) in the nuclei of infected hepatocytes. A complete cure from chronic HBV infection would require the elimination of this cccDNA from infected hepatocytes.

The review article Soriano et al 2017 Expert Opinion on Investigational Drugs Vol. 26, pp 843 describes the current status in drug development aiming to achieve either a functional cure of HBV or a complete cure. This article highlights some of the more than 30 drugs that are currently being tested in HBV therapy, also mentioning that any effective treatment leading to a cure will probably require a combination of a virus targeting therapy and an immunotherapy. Antisense oligonucleotides are essentially single stranded oligonucleotides capable of modulating expression of a target gene by hybridizing to a target nucleic acid. Target modulation can be down-regulation via RNase H mediated degradation or by blockage of the transcription. Antisense oligonucleotides can also up-regulate a target e.g. via splice switching or micro RNA repression. For targets in the liver GalNAc conjugation has proven very effective for delivering antisense oligonucleotides. WO 2014/179627 and WO2015/173208 describe HBV treatment through degradation of HBV mRNA in hepatocytes using single stranded antisense oligonucleotides in combination with GalNAc conjugation. Various combination therapies, including TLR7 agonist GS-9620, are briefly mentioned in WO2015/173208.

WO2016/077321 describes HBV treatment through degradation of HBV mRNA in hepatocytes using double stranded siRNA in combination with GalNAc conjugation on the sense strand. Various combination therapies including TLR7 agonists are briefly mentioned.

WO2017/157899 describes single-stranded LNA oligonucleotide conjugates for reducing PD-L1 expression. WO2019/079781 describes RNAi therapeutics targeting HBsAg.

To our knowledge no specific combinations of therapeutic oligonucleotides against HBV have been tested in vitro or in vivo in the art.

### OBJECTIVE OF THE INVENTION

The present invention identifies novel pharmaceutical combinations of HBV therapeutics, which provide an advantage over monotherapy treatments. In particular, the present invention identifies a novel pharmaceutical combination of an RNAi oligonucleotide targeting HBV and an anti-PDL1 antisense oligonucleotide, and advantageous dosage regimes thereof. The specific combination of an RNAi oligonucleotide targeting HBV and an anti-PDL1 antisense oligonucleotide obtains a surprising, synergistic effect on HBV serum markers, beyond that which could be expected for these individual therapeutics alone.

### SUMMARY OF INVENTION

The present invention is defined by the claims. The description provides further illustration of embodiments and alternatives according to the present invention.

In one aspect, the present invention provides pharmaceutical combinations comprising at least two HBV therapeutics. Herein, a HBV therapeutic is any drug or treatment that is useful against HBV infection. A HBV therapeutic may be in the form of an active ingredient, a prodrug, a composition, a conjugate or any other form that results in the realisation of the therapeutic effect of the drug when that form is administered to a patient.

In a preferred embodiment, the pharmaceutical combination comprises an RNAi oligonucleotide targeting HBV and an anti-PDL1 oligonucleotide.

In an embodiment, the pharmaceutical combination comprises the RNAi oligonucleotide targeting HBV defined herein as Therapeutic T1, and the anti-PDL1 antisense oligonucleotide defined herein as Therapeutic T2.

In a further aspect, the present invention provides a composition comprising the pharmaceutical combination of the invention. Preferably, in the pharmaceutical combination the RNAi oligonucleotide targeting HBV is comprised in a first composition and the anti-PDL1 antisense oligonucleotide is comprised in a second composition, optionally wherein any additional HBV therapeutic is comprised in a third composition.

In a further aspect, the present invention provides a kit of parts comprising a first HBV therapeutic comprised in the pharmaceutical combination as defined herein and instructions for administration with a second HBV therapeutic comprised in the pharmaceutical combination as defined herein to treat a hepatitis B virus infection. In some embodiments, the kit comprises both or all HBV therapeutics comprised in the combination.

In a further aspect, the present invention provides a use of the pharmaceutical combination, composition or kit of the invention for treating a hepatitis B virus infection.

In a further aspect, the present invention provides the pharmaceutical combination, composition or kit of the invention, for use in medicine.

In a further aspect, the present invention provides the pharmaceutical combination, composition or kit of the invention, for use in treatment of a hepatitis B virus infection.

In a further aspect, the present invention provides a use of the pharmaceutical combination, composition or kit of the invention in the manufacture of a medicament.

In a further aspect, the present invention provides a use of the pharmaceutical combination, composition or kit of the invention in the manufacture of a medicament for treating a hepatitis B virus infection.

In a further aspect, the present invention provides a method for treating a hepatitis B virus infection comprising administering a therapeutically effective amount of the pharmaceutical combination, composition or kit of the invention to a subject infected with a hepatitis B virus infection.

In a further aspect, the present invention provides a method of reducing expression of hepatitis B virus surface antigen in a cell, the method comprising delivering to the cell the pharmaceutical combination or composition of the invention.

The present invention further provides advantageous dosage regimes for administering the pharmaceutical combinations of the present invention.

### BRIEF DESCRIPTION OF FIGURES

FIG. 1 shows the serum levels of HBsAg (panel A) and the change in HBsAg serum levels (panel B) during the course of the study herein.

FIG. 2 shows the serum levels of HBeAg (panel A) and the change in HBeAg serum levels (panel B) during the course of the study herein.

FIG. 3 shows the serum levels of HBV-DNA (panel A) and the change in HBV-DNA serum levels (panel B) during the course of the study herein.

FIG. 4 shows the change in HBsAg, HBeAg and HBV-DNA serum levels during the course of the study herein, relative to Day 0 of the study. HBV siRNA surrogate=sT1, PDL1 LNA=sT2. The results for a study group that was administered a pharmaceutical combination of the invention (G10) are shown, in contrast with study groups that were administered a vehicle control (G1), an equivalent dose of the RNAi oligonucleotide targeting HBV as a monotherapy (G03) and an equivalent dose of the anti-PDL1 antisense oligonucleotide as a monotherapy (G06).

FIG. 5 shows a particular, specific definition of Therapeutic T1, the RNAi oligonucleotide targeting HBV to be used in preferred pharmaceutical combinations of the present invention.

FIG. 6 shows the results of Example 2, including the change in serum HBsAg and HBV-DNA levels upon administration of an equivalent surrogate Therapeutic T1 and Therapeutic T3 alone and in combination. A significant decrease in HBsAg and HBV-DNA is seen with the inventive combination of T1 and T3.

FIGS. 7 and 8 show the results of Example 3, including the change in serum HBsAg, HBV-DNA and HBeAg levels upon administration of an equivalent surrogate Therapeutic T1 and T5 as defined in Example 3. A significant decrease in serum markers is seen with the inventive combination of T1 and T5, in particular the combination significantly decreased serum levels of HBsAg and HBV-DNA during the T5 treatment period Day 21-33.

### DEFINITIONS

### Oligonucleotide

The term “oligonucleotide” as used herein is defined as it is generally understood by the skilled person as a molecule comprising two or more covalently linked nucleosides. Such covalently bound nucleosides may also be referred to as nucleic acid molecules or oligomers.

Oligonucleotides are commonly made in the laboratory by solid-phase chemical synthesis followed by purification and isolation. When referring to a sequence of the oligonucleotide, reference is made to the sequence or order of nucleobase moieties, or modifications thereof, of the covalently linked nucleotides or nucleosides. The oligonucleotide of the invention is man-made, and is chemically synthesized, and is typically purified or isolated. The oligonucleotide of the invention may comprise one or more modified nucleosides or nucleotides, such as 2′ sugar modified nucleosides.

Further, an oligonucleotide is a short nucleic acid, e.g., of less than 100 nucleotides in length. An oligonucleotide may be single-stranded or double-stranded. An oligonucleotide may or may not have duplex regions. As a set of non-limiting examples, an oligonucleotide may be, but is not limited to, a small interfering RNA (siRNA), microRNA (miRNA), short hairpin RNA (shRNA), dicer substrate interfering RNA (dsiRNA), antisense oligonucleotide, short siRNA, or single-stranded siRNA. In some embodiments, a double-stranded oligonucleotide is an RNAi oligonucleotide.

### Synthetic

As used herein, the term “synthetic” refers to a nucleic acid or other molecule that is artificially synthesized (e.g., using a machine (e.g., a solid state nucleic acid synthesizer)) or that is otherwise not derived from a natural source (e.g., a cell or organism) that normally produces the molecule.

### Double-Stranded Oligonucleotide

As used herein, the term “double-stranded oligonucleotide” refers to an oligonucleotide that is substantially in a duplex form. In some embodiments, complementary base-pairing of duplex region(s) of a double-stranded oligonucleotide is formed between antiparallel sequences of nucleotides of covalently separate nucleic acid strands. In some embodiments, complementary base-pairing of duplex region(s) of a double-stranded oligonucleotide is formed between antiparallel sequences of nucleotides of nucleic acid strands that are covalently linked. In some embodiments, complementary base-pairing of duplex region(s) of a double-stranded oligonucleotide is formed from a single nucleic acid strand that is folded (e.g., via a hairpin) to provide complementary antiparallel sequences of nucleotides that base pair together. In some embodiments, a double-stranded oligonucleotide comprises two covalently separate nucleic acid strands that are fully duplexed with one another. However, in some embodiments, a double-stranded oligonucleotide comprises two covalently separate nucleic acid strands that are partially duplexed, e.g., having overhangs at one or both ends. In some embodiments, a double-stranded oligonucleotide comprises antiparallel sequences of nucleotides that are partially complementary, and thus, may have one or more mismatches, which may include internal mismatches or end mismatches.

### Strand

As used herein, the term “strand” refers to a single contiguous sequence of nucleotides linked together through internucleotide linkages (e.g., phosphodiester linkages, phosphorothioate linkages). In some embodiments, a strand has two free ends, e.g., a 5′-end and a 3′-end.

### Duplex

As used herein, the term “duplex,” in reference to nucleic acids (e.g., oligonucleotides), refers to a structure formed through complementary base-pairing of two antiparallel sequences of nucleotides.

### Overhang

As used herein, the term “overhang” refers to terminal non-base pairing nucleotide(s) resulting from one strand or region extending beyond the terminus of a complementary strand with which the one strand or region forms a duplex. In some embodiments, an overhang comprises one or more unpaired nucleotides extending from a duplex region at the 5′ terminus or 3′ terminus of a double-stranded oligonucleotide. In certain embodiments, the overhang is a 3′ or 5′ overhang on the antisense strand or sense strand of a double-stranded oligonucleotide.

### Loop

As used herein, the term “loop” refers to a unpaired region of a nucleic acid (e.g., oligonucleotide) that is flanked by two antiparallel regions of the nucleic acid that are sufficiently complementary to one another, such that under appropriate hybridization conditions (e.g., in a phosphate buffer, in a cells), the two antiparallel regions, which flank the unpaired region, hybridize to form a duplex (referred to as a “stem”).

### RNAi Oligonucleotide

As used herein, the term “RNAi oligonucleotide” refers to either (a) a double stranded oligonucleotide having a sense strand (passenger) and antisense strand (guide), in which the antisense strand or part of the antisense strand is used by the Argonaute 2 (Ago2) endonuclease in the cleavage of a target mRNA or (b) a single stranded oligonucleotide having a single antisense strand, where that antisense strand (or part of that antisense strand) is used by the Ago2 endonuclease in the cleavage of a target mRNA.

### RNAi Agent

The terms ‘iRNA,” “RNAi agent,” ‘iRNA agent,” and “RNA interference agent” as used interchangeably herein, refer to an agent, e.g. an RNAi oligonucleotide, that contains RNA nucleosides herein and which mediates the targeted cleavage of an RNA transcript via an RNA-induced silencing complex (RISC) pathway. iRNA directs the sequence-specific degradation of mRNA through a process known as RNA interference (RNAi). The iRNA modulates, e.g. inhibits, the expression of the target nucleic acid in a cell, e.g. a cell within a subject, such as a mammalian subject. RNAi agents include single stranded RNAi agents and double stranded siRNAs, as well as short hairpin RNAs (shRNAs). The oligonucleotide of the invention or contiguous nucleotide sequence thereof may be in the form of an RNAi agent, or form part of an RNAi agent, such as a siRNA or shRNA. In some embodiments of the invention, the oligonucleotide of the invention or contiguous nucleotide sequence thereof is an RNAi agent, such as a siRNA.

siRNAs

The term siRNA refers to small interfering ribonucleic acid RNAi agents and is a class of double-stranded RNA molecules, also known in the art as short interfering RNA or silencing RNA. siRNAs typically comprise a sense strand (also referred to as a passenger strand) and an antisense strand (also referred to as the guide strand), wherein each strand are of 17-30 nucleotides in length, typically 19-25 nucleosides in length, wherein the antisense strand is complementary, such as fully complementary, to the target nucleic acid (suitably a mature mRNA sequence), and the sense strand is complementary to the antisense strand so that the sense strand and antisense strand form a duplex or duplex region. siRNA strands may form a blunt ended duplex, or advantageously the sense and antisense strand 3′ ends may form a 3′ overhang of e.g. 1, 2 or 3 nucleosides. In some embodiments, both the sense strand and antisense strand have a 2nt 3′ overhang. The duplex region may therefore be, for example 17-25 nucleotides in length, such as 21-23 nucleotide in length.

Once inside a cell the antisense strand is incorporated into the RISC complex which mediates target degradation or target inhibition of the target nucleic acid. siRNAs typically comprise modified nucleosides in addition to RNA nucleosides, or in some embodiments all of the nucleotides of an siRNA strand may be modified (the sense 2′ sugar modified nucleosides such as LNA (see WO2004083430, WO2007085485 for example), 2′-fluoro, 2′-O-methyl or 2′-O-methoxyethyl may be incorporated into siRNAs). In some embodiments the passenger stand of the siRNA may be discontinuous (see WO2007107162 for example). The incorporation of thermally destabilizing nucleotides occurring at a seed region of the antisense strand of siRNAs have been reported as useful in reducing off-target activity of siRNAs (see WO18098328 for example).

In some embodiments, the dsRNA agent, such as the siRNA of the invention, comprises at least one modified nucleotide. In some embodiments, substantially all of the nucleotides of the sense strand comprise a modification; substantially all of the nucleotides of the antisense strand comprise a modification; or substantially all of the nucleotides of the sense strand and substantially all of the nucleotides of the antisense strand comprise a modification. In yet other embodiments, all of the nucleotides of the sense strand comprise a modification; all of the nucleotides of the antisense strand comprise a modification; or all of the nucleotides of the sense strand and all of the nucleotides of the antisense strand comprise a modification. In some embodiments, the modified nucleotides may be independently selected from the group consisting of a deoxy-nucleotide, a 3′-terminal deoxy-thymine (dT) nucleotide, a 2′-O-methyl modified nucleotide, a 2′-fluoro modified nucleotide, a 2′-deoxy-modified nucleotide, a locked nucleotide, an unlocked nucleotide, a conformationally restricted nucleotide, a constrained ethyl nucleotide, an abasic nucleotide, a 2′-amino-modified nucleotide, a 2′-O-allyl-modified nucleotide, 2′-C-alkyl-modified nucleotide, 2′-hydroxyl-modified nucleotide, a 2′-methoxyethyl modified nucleotide, a 2′-O-alkyl-modified nucleotide, a morpholino nucleotide, a phosphoramidate, a non-natural base comprising nucleotide, an unlinked nucleotide, a tetrahydropyran modified nucleotide, a 1,5-anhydrohexitol modified nucleotide, a cyclohexenyl modified nucleotide, a nucleotide comprising a phosphorothioate group, a nucleotide comprising a methylphosphonate group, a nucleotide comprising a 5′-phosphate, a nucleotide comprising a 5′-phosphate mimic, a glycol modified nucleotide, and a 2-O—(N-methylacetamide) modified nucleotide, and combinations thereof. Suitably the siRNA comprises a 5′ phosphate group or a 5′-phosphate mimic at the 5′ end of the antisense strand. In some embodiments the 5′ end of the antisense strand is a RNA nucleoside.

In one embodiment, the dsRNA agent further comprises at least one phosphorothioate or methylphosphonate internucleotide linkage. The phosphorothioate or methylphosphonate internucleotide linkage may be at the 3′-terminus one or both strand (e.g., the antisense strand; or the sense strand); or the phosphorothioate or methylphosphonate internucleoside linkage may be at the 5′-terminus of one or both strands (e.g., the antisense strand; or the sense strand); or the phosphorothioate or methylphosphonate internucleoside linkage may be at the both the 5′- and 3′-terminus of one or both strands (e.g., the antisense strand; or the sense strand). In some embodiments the remaining internucleoside linkages are phosphodiester linkages.

The dsRNA agent may further comprise a ligand. In some embodiments, the ligand is conjugated to the 3′ end of the sense strand. For biological distribution, siRNAs may be conjugated to a targeting ligand, and/or be formulated into lipid nanoparticles, for example. Other aspects of the invention relate to pharmaceutical compositions comprising these dsRNA, such as siRNA molecules suitable for therapeutic use, and methods of inhibiting the expression of the target gene by administering the dsRNA molecules such as siRNAs of the invention, e.g., for the treatment of various disease conditions as disclosed herein.

### Tetraloop

As used herein, the term “tetraloop” refers to a loop that increases stability of an adjacent duplex formed by hybridization of flanking sequences of nucleotides. The increase in stability is detectable as an increase in melting temperature (Tₘ) of an adjacent stem duplex that is higher than the Tₘ of the adjacent stem duplex expected, on average, from a set of loops of comparable length consisting of randomly selected sequences of nucleotides. For example, a tetraloop can confer a melting temperature of at least 50° C., at least 55° C., at least 56° C., at least 58° C., at least 60° C., at least 65° C. or at least 75° C. in 10 mM NaHPO₄ to a hairpin comprising a duplex of at least 2 base pairs in length. In some embodiments, a tetraloop may stabilize a base pair in an adjacent stem duplex by stacking interactions. In addition, interactions among the nucleotides in a tetraloop include but are not limited to non-Watson-Crick base-pairing, stacking interactions, hydrogen bonding, and contact interactions (Cheong et al., Nature 1990 Aug. 16; 346(6285):680-2; Heus and Pardi, Science 1991 Jul. 12; 253(5016):191-4). In some embodiments, a tetraloop comprises 4 to 5 nucleotides. In certain embodiments, a tetraloop comprises or consists of three, four, five, or six nucleotides, which may or may not be modified (e.g., which may or may not be conjugated to a targeting moiety). In one embodiment, a tetraloop consists of four nucleotides. Any nucleotide may be used in the tetraloop and standard IUPAC-IUB symbols for such nucleotides may be used as described in Cornish-Bowden (1985) Nucl. Acids Res. 13: 3021-3030. For example, the letter “N” may be used to mean that any base may be in that position, the letter “R” may be used to show that A (adenine) or G (guanine) may be in that position, and “B” may be used to show that C (cytosine), G (guanine), or T (thymine) may be in that position. Examples of tetraloops include the UNCG family of tetraloops (e.g., UUCG), the GNRA family of tetraloops (e.g., GAAA), and the CUUG tetraloop (Woese et al., Proc Natl Acad Sci USA. 1990 November; 87(21):8467-71; Antao et al., Nucleic Acids Res. 1991 Nov. 11; 19(21):5901-5). Examples of DNA tetraloops include the d(GNNA) family of tetraloops (e.g., d(GTTA)), the d(GNRA) family of tetraloops, the d(GNAB) family of tetraloops, the d(CNNG) family of tetraloops, and the d(TNCG) family of tetraloops (e.g., d(TTCG)). See, for example: Nakano et al. Biochemistry, 41 (48), 14281-14292, 2002. SHINJI et al. Nippon Kagakkai Koen Yokoshu VOL. 78th; NO. 2; PAGE. 731 (2000), which are incorporated by reference herein for their relevant disclosures. In some embodiments, the tetraloop is contained within a nicked tetraloop structure.

### Nicked Tetraloop Structure

A “nicked tetraloop structure” is a structure of an RNAi oligonucleotide characterized by the presence of separate sense (passenger) and antisense (guide) strands, in which the sense strand has a region of complementarity with the antisense strand, and in which at least one of the strands, generally the sense strand, has a tetraloop configured to stabilize an adjacent stem region formed within the at least one strand.

### Antisense Oligonucleotides

The term “Antisense oligonucleotides” as used herein is defined as oligonucleotides capable of modulating expression of a target gene by hybridizing to a target nucleic acid, in particular to a contiguous sequence on a target nucleic acid. The antisense oligonucleotides are not essentially double stranded and are therefore not siRNAs or shRNAs. Preferably, the antisense oligonucleotides of the present invention are single stranded. It is understood that single stranded oligonucleotides of the present invention can form hairpins or intermolecular duplex structures (duplex between two molecules of the same oligonucleotide), as long as the degree of intra or inter self complementarity is less than 50% across of the full length of the oligonucleotide.

Advantageously, the single stranded antisense oligonucleotide of the invention does not contain RNA nucleosides, since this will decrease nuclease resistance.

Advantageously, the antisense oligonucleotide of the invention comprises one or more modified nucleosides or nucleotides, such as 2′ sugar modified nucleosides. Furthermore, it is advantageous that the nucleosides which are not modified are DNA nucleosides.

### Contiguous Nucleotide Sequence

The term “contiguous nucleotide sequence” refers to the region of the oligonucleotide which is complementary to the target nucleic acid. The term is used interchangeably herein with the term “contiguous nucleobase sequence” and the term “oligonucleotide motif sequence”. In some embodiments all the nucleotides of the oligonucleotide constitute the contiguous nucleotide sequence. In some embodiments the oligonucleotide comprises the contiguous nucleotide sequence, such as an F-G-F′ gapmer region, and may optionally comprise further nucleotide(s), for example a nucleotide linker region which may be used to attach a functional group to the contiguous nucleotide sequence. The nucleotide linker region may or may not be complementary to the target nucleic acid. It is understood that the contiguous nucleotide sequence of the oligonucleotide cannot be longer than the oligonucleotide as such and that the oligonucleotide cannot be shorter than the contiguous nucleotide sequence.

### Nucleotides

Nucleotides are the building blocks of oligonucleotides and polynucleotides, and for the purposes of the present invention include both naturally occurring and non-naturally occurring nucleotides. In nature, nucleotides, such as DNA and RNA nucleotides comprise a ribose sugar moiety, a nucleobase moiety and one or more phosphate groups (which is absent in nucleosides). Nucleosides and nucleotides may also interchangeably be referred to as “units” or “monomers”.

### Deoxyribonucleotide

As used herein, the term “deoxyribonucleotide” refers to a nucleotide having a hydrogen in place of a hydroxyl at the 2′ position of its pentose sugar as compared with a ribonucleotide. A modified deoxyribonucleotide is a deoxyribonucleotide having one or more modifications or substitutions of atoms other than at the 2′ position, including modifications or substitutions in or of the sugar, phosphate group or base.

### Ribonucleotide

As used herein, the term “ribonucleotide” refers to a nucleotide having a ribose as its pentose sugar, which contains a hydroxyl group at its 2′ position. A modified ribonucleotide is a ribonucleotide having one or more modifications or substitutions of atoms other than at the 2′ position, including modifications or substitutions in or of the ribose, phosphate group or base.

### Modified Nucleoside

The term “modified nucleoside” or “nucleoside modification” as used herein refers to nucleosides modified as compared to the equivalent DNA or RNA nucleoside by the introduction of one or more modifications of the sugar moiety or the (nucleo)base moiety. In a preferred embodiment the modified nucleoside comprise a modified sugar moiety. The term modified nucleoside may also be used herein interchangeably with the term “nucleoside analogue” or modified “units” or modified “monomers”. Nucleosides with an unmodified DNA or RNA sugar moiety are termed DNA or RNA nucleosides herein. Nucleosides with modifications in the base region of the DNA or RNA nucleoside are still generally termed DNA or RNA if they allow Watson Crick base pairing.

### Modified Nucleotide

As used herein, the term “modified nucleotide” refers to a nucleotide having one or more chemical modifications compared with a corresponding reference nucleotide selected from: adenine ribonucleotide, guanine ribonucleotide, cytosine ribonucleotide, uracil ribonucleotide, adenine deoxyribonucleotide, guanine deoxyribonucleotide, cytosine deoxyribonucleotide and thymidine deoxyribonucleotide. In some embodiments, a modified nucleotide is a non-naturally occurring nucleotide. In some embodiments, a modified nucleotide has one or more chemical modification in its sugar, nucleobase and/or phosphate group. In some embodiments, a modified nucleotide has one or more chemical moieties conjugated to a corresponding reference nucleotide. Typically, a modified nucleotide confers one or more desirable properties to a nucleic acid in which the modified nucleotide is present. For example, a modified nucleotide may improve thermal stability, resistance to degradation, nuclease resistance, solubility, bioavailability, bioactivity, reduced immunogenicity, etc.

### Modified Internucleoside Linkage

The term “modified internucleoside linkage” is defined as generally understood by the skilled person as linkages other than phosphodiester (PO) linkages, that covalently couples two nucleosides together. The oligonucleotides of the invention may therefore comprise modified internucleoside linkages. In some embodiments, the modified internucleoside linkage increases the nuclease resistance of the oligonucleotide compared to a phosphodiester linkage. For naturally occurring oligonucleotides, the internucleoside linkage includes phosphate groups creating a phosphodiester bond between adjacent nucleosides. Modified internucleoside linkages are particularly useful in stabilizing oligonucleotides for in vivo use, and may serve to protect against nuclease cleavage at regions of DNA or RNA nucleosides in the oligonucleotide of the invention, for example within the gap region G of a gapmer oligonucleotide, as well as in regions of modified nucleosides, such as region F and F′.

In an embodiment, the oligonucleotide comprises one or more internucleoside linkages modified from the natural phosphodiester, such as one or more modified internucleoside linkages that is for example more resistant to nuclease attack. Nuclease resistance may be determined by incubating the oligonucleotide in blood serum or by using a nuclease resistance assay (e.g. snake venom phosphodiesterase (SVPD)), both are well known in the art. Internucleoside linkages which are capable of enhancing the nuclease resistance of an oligonucleotide are referred to as nuclease resistant internucleoside linkages. In some embodiments at least 50% of the internucleoside linkages in the oligonucleotide, or contiguous nucleotide sequence thereof, are modified, such as at least 60%, such as at least 70%, such as at least 75%, such as at least 80% or such as at least 90% of the internucleoside linkages in the oligonucleotide, or contiguous nucleotide sequence thereof, are modified. In some embodiments all of the internucleoside linkages of the oligonucleotide, or contiguous nucleotide sequence thereof, are modified. It will be recognized that, in some embodiments the nucleosides which link the oligonucleotide of the invention to a non-nucleotide functional group, such as a conjugate, may be phosphodiester. In some embodiments all of the internucleoside linkages of the oligonucleotide, or contiguous nucleotide sequence thereof, are nuclease resistant internucleoside linkages.

With the oligonucleotides of the invention it is advantageous to use phosphorothioate internucleoside linkages.

Phosphorothioate internucleoside linkages are particularly useful due to nuclease resistance, beneficial pharmacokinetics and ease of manufacture. In some embodiments at least 50% of the internucleoside linkages in the oligonucleotide, or contiguous nucleotide sequence thereof, are phosphorothioate, such as at least 60%, such as at least 70%, such as at least 75%, such as at least 80% or such as at least 90% of the internucleoside linkages in the oligonucleotide, or contiguous nucleotide sequence thereof, are phosphorothioate. In some embodiments all of the internucleoside linkages of the oligonucleotide, or contiguous nucleotide sequence thereof, are phosphorothioate.

In some embodiments, the oligonucleotide of the invention comprises both phosphorothioate internucleoside linkages and at least one phosphodiester linkage, such as 2, 3 or 4 phosphodiester linkages, in addition to the phosphorodithioate linkage(s). In a gapmer oligonucleotide, phosphodiester linkages, when present, are suitably not located between contiguous DNA nucleosides in the gap region G.

Nuclease resistant linkages, such as phosphorothioate linkages, are particularly useful in oligonucleotide regions capable of recruiting nuclease when forming a duplex with the target nucleic acid, such as region G for gapmers. Phosphorothioate linkages may, however, also be useful in non-nuclease recruiting regions and/or affinity enhancing regions such as regions F and F′ for gapmers. Gapmer oligonucleotides may, in some embodiments comprise one or more phosphodiester linkages in region F or F′, or both region F and F′, where all the internucleoside linkages in region G may be phosphorothioate.

Advantageously, all the internucleoside linkages of the contiguous nucleotide sequence of the oligonucleotide are phosphorothioate, or all the internucleoside linkages of the oligonucleotide are phosphorothioate linkages. In particular, all the internucleoside linkages of the contiguous nucleotide sequence of the antisense oligonucleotide are phosphorothioate, or all the internucleoside linkages of the antisense oligonucleotide are phosphorothioate linkages. It is recognized that, as disclosed in EP 2 742 135, therapeutic oligonucleotides may comprise other internucleoside linkages (other than phosphodiester and phosphorothioate), for example alkyl phosphonate/methyl phosphonate internucleoside, which according to EP 2 742 135 may for example be tolerated in an otherwise DNA phosphorothioate the gap region.

### Nucleobase

The term nucleobase includes the purine (e.g. adenine and guanine) and pyrimidine (e.g. uracil, thymine and cytosine) moiety present in nucleosides and nucleotides which form hydrogen bonds in nucleic acid hybridization. In the context of the present invention the term nucleobase also encompasses modified nucleobases which may differ from naturally occurring nucleobases, but are functional during nucleic acid hybridization. In this context “nucleobase” refers to both naturally occurring nucleobases such as adenine, guanine, cytosine, thymidine, uracil, xanthine and hypoxanthine, as well as non-naturally occurring variants. Such variants are for example described in Hirao et al (2012) Accounts of Chemical Research vol 45 page 2055 and Bergstrom (2009) Current Protocols in Nucleic Acid Chemistry Suppl. 37 1.4.1.

In some embodiments the nucleobase moiety is modified by changing the purine or pyrimidine into a modified purine or pyrimidine, such as substituted purine or substituted pyrimidine, such as a nucleobase selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine and 2-chloro-6-aminopurine.

The nucleobase moieties may be indicated by the letter code for each corresponding nucleobase, e.g. A, T, G, C or U, wherein each letter may optionally include modified nucleobases of equivalent function. For example, in the exemplified oligonucleotides, the nucleobase moieties are selected from A, T, G, C, and 5-methyl cytosine. Optionally, for LNA gapmers, 5-methyl cytosine LNA nucleosides may be used.

### Modified Oligonucleotide

The term modified oligonucleotide describes an oligonucleotide comprising one or more sugar-modified nucleosides and/or modified internucleoside linkages. The term “chimeric” oligonucleotide is a term that has been used in the literature to describe oligonucleotides with modified nucleosides.

### Complementarity

As used herein, “complementary” refers to a structural relationship between two nucleotides (e.g., on two opposing nucleic acids or on opposing regions of a single nucleic acid strand), or between two sequences of nucleotides, that permits the two nucleotides, or two sequences of nucleotides, to form base pairs with one another. For example, a purine nucleotide of one nucleic acid that is complementary to a pyrimidine nucleotide of an opposing nucleic acid may base pair together by forming hydrogen bonds with one another. In some embodiments, complementary nucleotides can base pair in the Watson-Crick manner or in any other manner that allows for the formation of stable duplexes. Watson-Crick base pairs are guanine (G)-cytosine (C) and adenine (A)-thymine (T)/uracil (U). It will be understood that oligonucleotides may comprise nucleosides with modified nucleobases, for example 5-methyl cytosine is often used in place of cytosine, and as such the term complementarity encompasses Watson Crick base-paring between non-modified and modified nucleobases (see for example Hirao et al (2012) Accounts of Chemical Research vol 45 page 2055 and Bergstrom (2009) Current Protocols in Nucleic Acid Chemistry Suppl. 37 1.4.1).

The term “% complementary” as used herein, refers to the proportion of nucleotides (in percent) of a contiguous nucleotide sequence in a nucleic acid molecule (e.g. oligonucleotide) which across the contiguous nucleotide sequence, is complementary to a reference sequence (e.g. a target sequence or sequence motif). The percentage of complementarity is thus calculated by counting the number of aligned nucleobases that are complementary (from e.g. Watson Crick base pair) between the two sequences (when aligned with the target sequence 5′-3′ and the oligonucleotide sequence from 3′-5′), dividing that number by the total number of nucleotides in the oligonucleotide and multiplying by 100. In such a comparison a nucleobase/nucleotide which does not align (e.g. form a base pair) is termed a mismatch. Insertions and deletions are not allowed in the calculation of % complementarity of a contiguous nucleotide sequence. It will be understood that in determining complementarity, chemical modifications of the nucleobases are disregarded as long as the functional capacity of the nucleobase to form e.g. Watson Crick base pairing is retained (e.g. 5′-methyl cytosine is considered identical to a cytosine for the purpose of calculating % identity).

The term “fully complementary”, refers to 100% complementarity.

In some embodiments, two nucleic acids may have regions of multiple nucleotides that are complementary with each other so as to form regions of complementarity, as described herein.

### Region of Complementarity

As used herein, the term “region of complementarity” refers to a sequence of nucleotides of a nucleic acid (e.g., a double-stranded oligonucleotide) that is sufficiently complementary to an antiparallel sequence of nucleotides to permit hybridization between the two sequences of nucleotides under appropriate hybridization conditions, e.g., in a phosphate buffer, in a cell, etc.

### Identity

The term “Identity” as used herein, refers to the proportion of nucleotides (expressed in percent) of a contiguous nucleotide sequence in a nucleic acid molecule (e.g. oligonucleotide) which across the contiguous nucleotide sequence, is identical to a reference sequence (e.g. a sequence motif). The percentage of identity is thus calculated by counting the number of aligned nucleobases that are identical (a Match) between two sequences (in the contiguous nucleotide sequence of the compound of the invention and in the reference sequence), dividing that number by the total number of nucleotides in the oligonucleotide and multiplying by 100. Therefore, Percentage of Identity=(Matches×100)/Length of aligned region (e.g. the contiguous nucleotide sequence). Insertions and deletions are not allowed in the calculation of the percentage of identity of a contiguous nucleotide sequence. It will be understood that in determining identity, chemical modifications of the nucleobases are disregarded as long as the functional capacity of the nucleobase to form Watson Crick base pairing is retained (e.g. 5-methyl cytosine is considered identical to a cytosine for the purpose of calculating % identity).

### Hybridization

The term “hybridizing” or “hybridizes” as used herein is to be understood as two nucleic acid strands (e.g. an oligonucleotide and a target nucleic acid) forming hydrogen bonds between base pairs on opposite strands thereby forming a duplex. The affinity of the binding between two nucleic acid strands is the strength of the hybridization. It is often described in terms of the melting temperature (Tₘ) defined as the temperature at which half of the oligonucleotides are duplexed with the target nucleic acid. At physiological conditions Tₘ is not strictly proportional to the affinity (Mergny and Lacroix, 2003, Oligonucleotides 13:515-537). The standard state Gibbs free energy ΔG° is a more accurate representation of binding affinity and is related to the dissociation constant (K_{d}) of the reaction by ΔG°=−RT ln(K_{d}), where R is the gas constant and T is the absolute temperature. Therefore, a very low ΔG° of the reaction between an oligonucleotide and the target nucleic acid reflects a strong hybridization between the oligonucleotide and target nucleic acid. ΔG° is the energy associated with a reaction where aqueous concentrations are 1M, the pH is 7, and the temperature is 37° C. The hybridization of oligonucleotides to a target nucleic acid is a spontaneous reaction and for spontaneous reactions ΔG° is less than zero. ΔG° can be measured experimentally, for example, by use of the isothermal titration calorimetry (ITC) method as described in Hansen et al., 1965, Chem. Comm. 36-38 and Holdgate et al., 2005, Drug Discov Today. The skilled person will know that commercial equipment is available for ΔG° measurements. ΔG° can also be estimated numerically by using the nearest neighbor model as described by SantaLucia, 1998, Proc Natl Acad Sci USA. 95: 1460-1465 using appropriately derived thermodynamic parameters described by Sugimoto et al., 1995, Biochemistry 34:11211-11216 and McTigue et al., 2004, Biochemistry 43:5388-5405. In order to have the possibility of modulating its intended nucleic acid target by hybridization, oligonucleotides of the present invention hybridize to a target nucleic acid with estimated ΔG° values below −10 kcal for oligonucleotides that are 10-30 nucleotides in length. In some embodiments the degree or strength of hybridization is measured by the standard state Gibbs free energy ΔG°. The oligonucleotides may hybridize to a target nucleic acid with estimated ΔG° values below the range of −10 kcal, such as below −15 kcal, such as below −20 kcal and such as below −25 kcal for oligonucleotides that are 8-30 nucleotides in length. In some embodiments the oligonucleotides hybridize to a target nucleic acid with an estimated ΔG° value of −10 to −60 kcal, such as −12 to −40, such as from −15 to −30 kcal or −16 to −27 kcal such as −18 to −25 kcal.

### Target Nucleic Acid

According to the present invention, the target nucleic acid may for example be a gene, a RNA, mRNA, viral mRNA or a cDNA sequence.

For in vivo or in vitro application, the oligonucleotide of the invention is typically capable of inhibiting the expression of the HBV target nucleic acid in a cell which is expressing the HBV target nucleic acid. The contiguous sequence of nucleobases of the oligonucleotide of the invention is typically complementary to the HBV target nucleic acid, as measured across the length of the oligonucleotide, optionally with the exception of one or two mismatches, and optionally excluding nucleotide based linker regions which may link the oligonucleotide to an optional functional group such as a conjugate, or other non-complementary terminal nucleotides (e.g. region D′ or D″).

### Target Sequence

The term “target sequence” as used herein refers to a sequence of nucleotides present in the target nucleic acid which comprises the nucleobase sequence which is complementary to the oligonucleotide of the invention. In some embodiments, the target sequence consists of a region on the target nucleic acid with a nucleobase sequence that is complementary to the contiguous nucleotide sequence of the oligonucleotide of the invention. This region of the target nucleic acid may interchangeably be referred to as the target nucleotide sequence, target sequence or target region. In some embodiments the target sequence is longer than the complementary sequence of a single oligonucleotide, and may, for example represent a preferred region of the target nucleic acid which may be targeted by several oligonucleotides of the invention.

### Target Cell

The term a “target cell” as used herein refers to a cell which is expressing the target nucleic acid. In some embodiments the target cell may be in vivo or in vitro. In some embodiments the target cell is a HBV infected mammalian cell such as a rodent cell, such as a mouse cell or a human cell, in particular a HBV infected hepatocyte.

In preferred embodiments the target cell expresses HBV mRNA and secretes HBsAg and HBeAg.

### Hepatocyte

As used herein, the term “hepatocyte” or “hepatocytes” refers to cells of the parenchymal tissues of the liver. These cells make up approximately 70-85% of the liver's mass and manufacture serum albumin, fibrinogen, and the prothrombin group of clotting factors (except for Factors 3 and 4). Markers for hepatocyte lineage cells may include, but are not limited to: transthyretin (Ttr), glutamine synthetase (GluI), hepatocyte nuclear factor 1a (Hnf1a), and hepatocyte nuclear factor 4a (Hnf4a). Markers for mature hepatocytes may include, but are not limited to: cytochrome P450 (Cyp3a11), fumarylacetoacetate hydrolase (Fah), glucose 6-phosphate (G6p), albumin (Alb), and OC2-2F8. See, e.g., Huch et al., (2013), Nature, 494(7436): 247-250, the contents of which relating to hepatocyte markers is incorporated herein by reference.

### Reduced Expression

As used herein, the term “reduced expression” of a gene refers to a decrease in the amount of RNA transcript or protein encoded by the gene and/or a decrease in the amount of activity of the gene in a cell or subject, as compared to an appropriate reference cell or subject. For example, the act of treating a cell with a pharmaceutical combination or a double-stranded oligonucleotide (e.g., one having an antisense strand that is complementary to an HBsAg mRNA sequence) may result in a decrease in the amount of RNA transcript, protein and/or enzymatic activity (e.g., encoded by the S gene of an HBV genome) compared to a cell that is not treated with the pharmaceutical combination or double-stranded oligonucleotide respectively. Similarly, “reducing expression” as used herein refers to an act that results in reduced expression of a gene (e.g., the S gene of an HBV genome).

### Naturally Occurring Variant

The term “naturally occurring variant thereof” refers to variants of the target nucleic acid which exist naturally within the defined taxonomic group, such as HBV genotypes A-H. Typically, when referring to “naturally occurring variants” of a polynucleotide the term may also encompass any allelic variant of the target sequence encoding genomic DNA which are found by chromosomal translocation or duplication, and the RNA, such as mRNA derived therefrom. “Naturally occurring variants” may also include variants derived from alternative splicing of the target sequence mRNA. When referenced, e.g. to a specific polypeptide sequence, the term also includes naturally occurring forms of the protein which may therefore be processed, e.g. by co- or post-translational modifications, such as signal peptide cleavage, proteolytic cleavage, glycosylation, etc.

### High Affinity Modified Nucleosides

A high affinity modified nucleoside is a modified nucleotide which, when incorporated into the oligonucleotide, enhances the affinity of the oligonucleotide for its complementary target, for example as measured by the melting temperature (Tₘ). A high affinity modified nucleoside of the present invention preferably result in an increase in melting temperature between +0.5 to +12° C., more preferably between +1.5 to +10° C. and most preferably between +3 to +8° C. per modified nucleoside. Numerous high affinity modified nucleosides are known in the art and include for example, many 2′ substituted nucleosides as well as locked nucleic acids (LNA) (see e.g. Freier & Altmann; Nucl. Acid Res., 1997, 25, 4429-4443 and Uhlmann; Curr. Opinion in Drug Development, 2000, 3(2), 293-213).

### Sugar Modifications

The oligomer of the invention may comprise one or more nucleosides which have a modified sugar moiety, i.e. a modification of the sugar moiety when compared to the ribose sugar moiety found in DNA and RNA.

Numerous nucleosides with modification of the ribose sugar moiety have been made, primarily with the aim of improving certain properties of oligonucleotides, such as affinity and/or nuclease resistance.

Such modifications include those where the ribose ring structure is modified, e.g. by replacement with a hexose ring (HNA), or a bicyclic ring, which typically have a biradicle bridge between the C2 and C4 carbons on the ribose ring (LNA), or an unlinked ribose ring which typically lacks a bond between the C2 and C3 carbons (e.g. UNA). Other sugar modified nucleosides include, for example, bicyclohexose nucleic acids (WO2011/017521) or tricyclic nucleic acids (WO2013/154798). Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′-OH group naturally found in DNA and RNA nucleosides. Substituents may, for example be introduced at the 2′, 3′, 4′ or 5′ positions.

### 2′ Sugar Modified Nucleosides

A 2′ sugar modified nucleoside is a nucleoside which has a substituent other than H or —OH at the 2′ position (2′ substituted nucleoside) or comprises a 2′ linked biradicle capable of forming a bridge between the 2′ carbon and a second carbon in the ribose ring, such as LNA (2′-4′ biradicle bridged) nucleosides.

Indeed, much focus has been spent on developing 2′ sugar substituted nucleosides, and numerous 2′ substituted nucleosides have been found to have beneficial properties when incorporated into oligonucleotides. For example, the 2′ modified sugar may provide enhanced binding affinity and/or increased nuclease resistance to the oligonucleotide. Examples of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleoside. For further examples, please see e.g. Freier & Altmann; Nucl. Acid Res., 1997, 25, 4429-4443 and Uhlmann; Curr. Opinion in Drug Development, 2000, 3(2), 293-213, and Deleavey and Damha, Chemistry and Biology 2012, 19, 937. Below are illustrations of some 2′ substituted modified nucleosides.

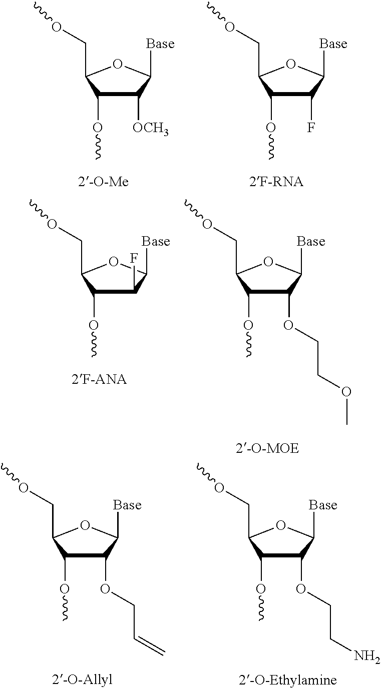

In relation to the present invention 2′ substituted sugar modified nucleosides does not include 2′ bridged nucleosides like LNA.

### Locked Nucleic Acid Nucleosides (LNA Nucleoside)

A “LNA nucleoside” is a 2′-modified nucleoside which comprises a biradical linking the C2′ and C4′ of the ribose sugar ring of said nucleoside (also referred to as a “2′-4′ bridge”), which restricts or locks the conformation of the ribose ring. These nucleosides are also termed bridged nucleic acid or bicyclic nucleic acid (BNA) in the literature. The locking of the conformation of the ribose is associated with an enhanced affinity of hybridization (duplex stabilization) when the LNA is incorporated into an oligonucleotide for a complementary RNA or DNA molecule. This can be routinely determined by measuring the melting temperature of the oligonucleotide/complement duplex.

Non limiting, exemplary LNA nucleosides are disclosed in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med. Chem. Lett. 12, 73-76, Seth et al. J. Org. Chem. 2010, Vol 75(5) pp. 1569-81, and Mitsuoka et al., Nucleic Acids Research 2009, 37(4), 1225-1238, and Wan and Seth, J. Medical Chemistry 2016, 59, 9645-9667. Further non limiting, exemplary LNA nucleosides are disclosed in Scheme 1.

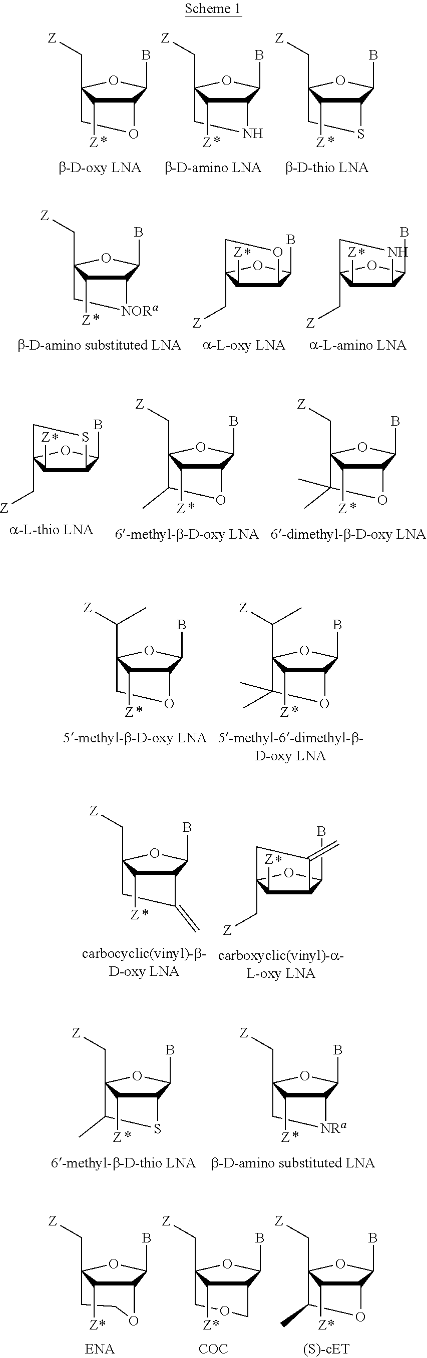

Particular LNA nucleosides are beta-D-oxy-LNA, 6′-methyl-beta-D-oxy LNA such as (S)-6′-methyl-beta-D-oxy-LNA (ScET) and ENA. A particularly advantageous LNA is beta-D-oxy-LNA.

### Phosphate Analog

As used herein, the term “phosphate analog” refers to a chemical moiety that mimics the electrostatic and/or steric properties of a phosphate group. In some embodiments, a phosphate analog is positioned at the 5′ terminal nucleotide of an oligonucleotide in place of a 5′-phosphate, which is often susceptible to enzymatic removal. In some embodiments, a 5′ phosphate analog contains a phosphatase-resistant linkage. Examples of phosphate analogs include 5′ phosphonates, such as 5′ methylenephosphonate (5′-MP) and 5′-(E)-vinylphosphonate (5′-VP). In some embodiments, an oligonucleotide has a phosphate analog at a 4′-carbon position of the sugar (referred to as a “4′-phosphate analog”) at a 5′-terminal nucleotide. An example of a 4′-phosphate analog is oxymethylphosphonate, in which the oxygen atom of the oxymethyl group is bound to the sugar moiety (e.g., at its 4′-carbon) or analog thereof. See, for example, U.S. Provisional Application Nos. 62/383,207, filed on Sep. 2, 2016, and 62/393,401, filed on Sep. 12, 2016, the contents of each of which relating to phosphate analogs are incorporated herein by reference. Other modifications have been developed for the 5′ end of oligonucleotides (see, e.g., WO 2011/133871; U.S. Pat. No. 8,927,513; and Prakash et al. (2015), Nucleic Acids Res., 43(6):2993-3011, the contents of each of which relating to phosphate analogs are incorporated herein by reference).

### Nuclease Mediated Degradation

Nuclease mediated degradation refers to an oligonucleotide capable of mediating degradation of a complementary nucleotide sequence when forming a duplex with such a sequence.

In some embodiments, the antisense oligonucleotide may function via nuclease mediated degradation of the target nucleic acid, where the oligonucleotides of the invention are capable of recruiting a nuclease, particularly an endonuclease, preferably an endoribonuclease (RNase), such as RNase H. Examples of oligonucleotide designs which operate via nuclease mediated mechanisms are oligonucleotides which typically comprise a region of at least 5 or 6 consecutive DNA nucleosides and are flanked on one side or both sides by affinity enhancing nucleosides, for example gapmers, headmers and tailmers.

### RNase H Activity and Recruitment

In one embodiment, the therapeutic oligonucleotide is an antisense oligonucleotide capable of recruiting RNase H. The RNase H activity of an antisense oligonucleotide refers to its ability to recruit RNase H when in a duplex with a complementary RNA molecule. WO01/23613 provides in vitro methods for determining RNase H activity, which may be used to determine the ability to recruit RNase H. Typically an oligonucleotide is deemed capable of recruiting RNase H if it, when provided with a complementary target nucleic acid sequence, has an initial rate, as measured in pmol/l/min, of at least 5%, such as at least 10% or more than 20% of the of the initial rate determined when using a oligonucleotide having the same base sequence as the modified oligonucleotide being tested, but containing only DNA monomers with phosphorothioate linkages between all monomers in the oligonucleotide, and using the methodology provided by Example 91-95 of WO01/23613 (hereby incorporated by reference).

For use in determining RNase H activity, recombinant human RNase H1 is available from Lubio Science GmbH, Lucerne, Switzerland.

### Gapmer

In some embodiments where the therapeutic oligonucleotide of the present invention is an antisense oligonucleotide, the nucleic acid molecule of the invention, or contiguous nucleotide sequence thereof are gapmer antisense oligonucleotides. The antisense gapmers are commonly used to inhibit a target nucleic acid via RNase H mediated degradation. In an embodiment of the invention the antisense oligonucleotide of the invention is capable of recruiting RNase H.

A gapmer antisense oligonucleotide comprises at least three distinct structural regions: a 5′-flank, a gap and a 3′-flank, F-G-F′ in the '5->3′ orientation. The “gap” region (G) comprises a stretch of contiguous DNA nucleotides which enable the oligonucleotide to recruit RNase H. The gap region is flanked by a 5′ flanking region (F) comprising one or more sugar modified nucleosides, advantageously high affinity sugar modified nucleosides, and by a 3′ flanking region (F′) comprising one or more sugar modified nucleosides, advantageously high affinity sugar modified nucleosides. The one or more sugar modified nucleosides in region F and F′ enhance the affinity of the oligonucleotide for the target nucleic acid (i.e. are affinity enhancing sugar modified nucleosides). In some embodiments, the one or more sugar modified nucleosides in region F and F′ are 2′ sugar modified nucleosides, such as high affinity 2′ sugar modifications, such as independently selected from LNA and 2′-MOE.

In a gapmer design, the 5′ and 3′ most nucleosides of the gap region are DNA nucleosides, and are positioned adjacent to a sugar modified nucleoside of the 5′ (F) or 3′ (F′) region respectively.

The flanks may further be defined by having at least one sugar modified nucleoside at the end most distant from the gap region, i.e. at the 5′ end of the 5′ flank and at the 3′ end of the 3′ flank.

Regions F-G-F′ form a contiguous nucleotide sequence. Antisense oligonucleotides of the invention, or the contiguous nucleotide sequence thereof, may comprise a gapmer region of formula F-G-F′.

The overall length of the gapmer design F-G-F′ may be, for example 12 to 30 nucleosides, such as 13 to 24, such as 14 to 22 nucleosides, Such as from 13 to 17, such as 14 to 16 nucleosides.

By way of example, the gapmer oligonucleotide of the present invention can be represented by the following formulae:

F₁₋₆-G₆₋₁₆-F′₁₋₆, such as

F₁₋₄-G₇₋₁₀-F′₂₋₄

with the proviso that the overall length of the gapmer regions F-G-F′ is at least 12, such as at least 13 nucleotides in length.

In an aspect of the invention the antisense oligonucleotide or contiguous nucleotide sequence thereof consists of or comprises a gapmer of formula 5‘-F-G-F’-3′, where region F and F′ independently comprise or consist of 1-8 nucleosides, of which 1-4 are 2′ sugar modified and defines the 5′ and 3′ end of the F and F′ region, and G is a region of between 6 and 16 nucleosides which are capable of recruiting RNase H.

In one embodiment of the invention the contiguous nucleotide sequence is a gapmer of formula 5‘-F-G-F’-3′, where region F and F′ independently consist of 2-4 2′ sugar modified nucleotides and defines the 5′ and 3′ end of the F and F′ region, and G is a region between 6 and 10 DNA nucleosides which are capable of recruiting RNase H.

In some embodiments the gap region G may consist of 6, 7, 8, 9, 10, 11, 12, 13, 14, 15 or 16 contiguous phosphorothioate linked DNA nucleosides. In some embodiments the gap region G consist of 7 to 10 DNA nucleosides. In some embodiments, all internucleoside linkages in the gap are phosphorothioate linkages.

In some embodiments, region F and F′ independently consists of or comprises a contiguous sequence of sugar modified nucleosides. In some embodiments, the sugar modified nucleosides of region F may be independently selected from 2′-O-alkyl-RNA units, 2′-O-methyl-RNA, 2′-amino-DNA units, 2′-fluoro-DNA units, 2′-alkoxy-RNA, MOE units, LNA units, arabino nucleic acid (ANA) units and 2′-fluoro-ANA units.

In some embodiments, all the nucleosides of region F or F′, or F and F′ are LNA nucleosides, such as independently selected from beta-D-oxy LNA, ENA or ScET nucleosides. In some embodiments region F consists of 1-5, such as 2-4, such as 3-4 such as 1, 2, 3, 4 or 5 contiguous LNA nucleosides. In some embodiments, all the nucleosides of region F and F′ are beta-D-oxy LNA nucleosides.

In some embodiments, all the nucleosides of region F or F′, or F and F′ are 2′ substituted nucleosides, such as OMe or MOE nucleosides. In some embodiments region F consists of 1, 2, 3, 4, 5, 6, 7, or 8 contiguous OMe or MOE nucleosides. In some embodiments only one of the flanking regions can consist of 2′ substituted nucleosides, such as OMe or MOE nucleosides. In some embodiments it is the 5′ (F) flanking region that consists 2′ substituted nucleosides, such as OMe or MOE nucleosides whereas the 3′ (F′) flanking region comprises at least one LNA nucleoside, such as beta-D-oxy LNA nucleosides or cET nucleosides. In some embodiments it is the 3′ (F′) flanking region that consists 2′ substituted nucleosides, such as OMe or MOE nucleosides whereas the 5′ (F) flanking region comprises at least one LNA nucleoside, such as beta-D-oxy LNA nucleosides or cET nucleosides.

Further gapmer designs are disclosed in WO2004/046160, WO2007/146511 and WO2008/113832, hereby incorporated by reference.

### LNA Gapmer

An LNA gapmer is a gapmer wherein either one or both of region F and F′ comprises or consists of LNA nucleosides. A beta-D-oxy gapmer is a gapmer wherein either one or both of region F and F′ comprises or consists of beta-D-oxy LNA nucleosides.

In some embodiments the LNA gapmer is of formula: [LNA]₁₋₅-[region G]₆₋₁₀-[LNA]₁₋₅, wherein region G is as defined in the Gapmer region G definition.

### MOE Gapmers

A MOE gapmers is a gapmer wherein regions F and F′ consist of MOE nucleosides. In some embodiments the MOE gapmer is of design [MOE]₁₋₈-[Region G]₅₋₁₆-[MOE]₁₋₈, such as [MOE]₂-7-[Region G]₆₋₁₄-[MOE]₂₋₇, such as [MOE]₃₋₆-[Region G]₈₋₁₂-[MOE]₃₋₆, wherein region G is as defined in the Gapmer definition. MOE gapmers with a 5-10-5 design (MOE-DNA-MOE) have been widely used in the art.

### Mixed Wing Gapmer

A mixed wing gapmer is an LNA gapmer wherein one or both of region F and F′ comprise a 2′ substituted nucleoside, such as a 2′ substituted nucleoside independently selected from the group consisting of 2′-O-alkyl-RNA units, 2′-O-methyl-RNA, 2′-amino-DNA units, 2′-fluoro-DNA units, 2′-alkoxy-RNA, MOE units, arabino nucleic acid (ANA) units and 2′-fluoro-ANA units, such as MOE nucleosides. In some embodiments wherein at least one of region F and F′, or both region F and F′ comprise at least one LNA nucleoside, the remaining nucleosides of region F and F′ are independently selected from the group consisting of MOE and LNA. In some embodiments wherein at least one of region F and F′, or both region F and F′ comprise at least two LNA nucleosides, the remaining nucleosides of region F and F′ are independently selected from the group consisting of MOE and LNA. In some mixed wing embodiments, one or both of region F and F′ may further comprise one or more DNA nucleosides.

Mixed wing gapmer designs are disclosed in WO2008/049085 and WO2012/109395, both of which are hereby incorporated by reference.

### Region D′ or D″ in an Oligonucleotide

The oligonucleotide of the invention may in some embodiments comprise or consist of the contiguous nucleotide sequence of the oligonucleotide which is complementary to the target nucleic acid, such as the gapmer F-G-F′, and further 5′ and/or 3′ nucleosides. The further 5′ and/or 3′ nucleosides may or may not be fully complementary to the target nucleic acid. Such further 5′ and/or 3′ nucleosides may be referred to as region D′ and D″ herein.

The addition of region D′ or D″ may be used for the purpose of joining the contiguous nucleotide sequence, such as the gapmer, to a conjugate moiety or another functional group. When used for joining the contiguous nucleotide sequence with a conjugate moiety it can serve as a biocleavable linker. Alternatively, it may be used to provide exonuclease protection or for ease of synthesis or manufacture.

Region D′ and D″ can be attached to the 5′ end of region F or the 3′ end of region F′, respectively to generate designs of the following formulas D′-F-G-F′, F-G-F′-D″ or D′-F-G-F′-D″. In this instance the F-G-F′ is the gapmer portion of the oligonucleotide and region D′ or D″ constitute a separate part of the oligonucleotide. The transition between region D′ and F region and between region F′ and D″ region is characterized by a nucleoside with a phosphodiester linkage towards the D′ or D″ region and a phosphorothioate linkage towards the F or F′ region, and the nucleoside is considered to be a part of the gapmer (contiguous nucleotide sequence which is complementary to the target nucleic acid).

Region D′ or D″ may independently comprise or consist of 1, 2, 3, 4 or 5 additional nucleotides, which may be complementary or non-complementary to the target nucleic acid. The nucleotide adjacent to the F or F′ region is not a sugar-modified nucleotide, such as a DNA or RNA or base modified versions of these. The D′ or D″ region may serve as a nuclease susceptible biocleavable linker (see definition of linkers). In some embodiments the additional 5′ and/or 3′ end nucleotides are linked with phosphodiester linkages, and are DNA or RNA. Nucleotide based biocleavable linkers suitable for use as region D′ or D″ are disclosed in WO2014/076195, which include by way of example a phosphodiester linked DNA dinucleotide. In some embodiments region D′ or D″ is not complementary to or comprises at least 50% mismatches to the target nucleic acid.

In some embodiments region D′ or D″ comprises or consists of a dinucleotide of sequence AA, AT, AC, AG, TA, TT, TC, TG, CA, CT, CC, CG, GA, GT, GC, or GG, wherein C may be 5-methylcytosine, and/or T may be replaced with U. The internucleoside linkage in the dinucleotide is a phosphodiester linkage. In some embodiments region D′ or D″ comprises or consists of a trinucleotide of sequence AAA, AAT, AAC, AAG, ATA, ATT, ATC, ATG, ACA, ACT, ACC, ACG, AGA, AGT, AGC, AGG, TAA, TAT, TAC, TAG, TTA, TTT, TTC, TAG, TCA, TCT, TCC, TCG, TGA, TGT, TGC, TGG, CAA, CAT, CAC, CAG, CTA, CTG, CTC, CTT, CCA, CCT, CCC, CCG, CGA, CGT, CGC, CGG, GAA, GAT, GAC, CAG, GTA, GTT, GTC, GTG, GCA, GCT, GCC, GCG, GGA, GGT, GGC, and GGG wherein C may be 5-methylcytosine and/or T may be replaced with U. The internucleoside linkages are phosphodiester linkages. It will be recognized that when referring to (naturally occurring) nucleobases A (adenine, T (thymine), U (uracil), G (guanine), C (cytosine), these may be substituted with nucleobase analogues which function as the equivalent natural nucleobase (e.g. base pair with the complementary nucleoside).

In one embodiment the antisense oligonucleotide of the invention comprises a region D′ and/or D″ in addition to the contiguous nucleotide sequence which constitutes the gapmer.

In some embodiments, the antisense oligonucleotide of the present invention can be represented by the following formulae:

D′-F-G-F′, in particular D′₁₋₃-F₁₋₄-G₆₋₁₀-F′₂₋₄

F-G-F′-D″, in particular F₁₋₄-G₆₋₁₀-F′₂₋₄-D″₁₋₃

D′-F-G-F′-D″, in particular D′₁₋₃-F₁₋₄-G₆₋₁₀-F′₂₋₄-D″₁₋₃

In some embodiments the internucleoside linkage positioned between region D′ and region F is a phosphodiester linkage. In some embodiments the internucleoside linkage positioned between region F′ and region D″ is a phosphodiester linkage.

### Conjugate

The term conjugate as used herein refers to a non-nucleotide moiety (conjugate), such as a GalNAc cluster, which can be covalently linked to a therapeutic oligonucleotide. The term conjugate and cluster or conjugate moiety may be used interchangeably. In some instances the conjugated therapeutic oligonucleotide may also be termed an oligonucleotide conjugate. In an embodiment, the conjugate is a targeting ligand.

### Targeting Ligand

As used herein, the term “targeting ligand” refers to a molecule (e.g., a carbohydrate, amino sugar, cholesterol, polypeptide or lipid) that selectively binds to a cognate molecule (e.g., a receptor) of a tissue or cell of interest and that is conjugatable to another substance for purposes of targeting the other substance to the tissue or cell of interest. For example, in some embodiments, a targeting ligand may be conjugated to an oligonucleotide for purposes of targeting the oligonucleotide to a specific tissue or cell of interest. In some embodiments, a targeting ligand selectively binds to a cell surface receptor. Accordingly, in some embodiments, a targeting ligand when conjugated to an oligonucleotide facilitates delivery of the oligonucleotide into a particular cell through selective binding to a receptor expressed on the surface of the cell and endosomal internalization by the cell of the complex comprising the oligonucleotide, targeting ligand and receptor. In some embodiments, a targeting ligand is conjugated to an oligonucleotide via a linker that is cleaved following or during cellular internalization such that the oligonucleotide is released from the targeting ligand in the cell.

### Oligonucleotide Linkers

A linkage or linker is a connection between two atoms that links one chemical group or segment of interest to another chemical group or segment of interest via one or more covalent bonds. Conjugate groups can be attached to the oligonucleotide directly or through a linking moiety (e.g. linker or tether). Linkers serve to covalently connect a conjugate group, to an oligonucleotide or contiguous nucleotide sequence complementary to the target nucleic acid. In some embodiments of the invention the therapeutic oligonucleotide may optionally comprise a linker region which is positioned between the oligonucleotide or contiguous nucleotide sequence complementary to the target nucleic acid and the conjugate.

Such linkers can be biocleavable linkers comprising or consisting of a physiologically labile bond that is cleavable under conditions normally encountered or analogous to those encountered within a mammalian body. In one embodiment the biocleavable linker is susceptible to S1 nuclease cleavage.

For biocleavable linkers placed between the conjugate and the therapeutic oligonucleotide, it is preferred that the cleavage rate seen in the target tissue (for example muscle, liver, kidney or a tumor) is greater than that found in blood serum. In some embodiments, the biocleavable linker is at least about 20% cleaved, such as at least about 30% cleaved, such as at least about 40% cleaved, such as at least about 50% cleaved, such as at least about 60% cleaved, such as at least about 70% cleaved, such as at least about 75% cleaved when compared against a standard.

In a preferred embodiment the nuclease susceptible linker comprises between 1 and 10 nucleosides, such as 1, 2, 3, 4, 5, 6, 7, 8, 9 or 10 nucleosides, more preferably between 2 and 6 nucleosides and most preferably between 2 and 4 linked nucleosides comprising at least two consecutive phosphodiester linkages, such as at least 3 or 4 or 5 consecutive phosphodiester linkages. Preferably the nucleosides are DNA or RNA. Phosphodiester containing biocleavable linkers (PO linkers) are described in more detail in WO 2014/076195 (hereby incorporated by reference).

Additional or alternative linkers that are not necessarily biocleavable but primarily serve to covalently connect a conjugate to the oligonucleotide may also be used either alone or in combination with PO linkers. The non-cleavable linkers may comprise a chain structure or an oligomer of repeating units such as ethylene glycol, amino acid units or amino alkyl groups. In some embodiments the non-cleavable linker is an amino alkyl, such as a C2-C36 amino alkyl group, including, for example C6 to C12 amino alkyl groups. In a preferred embodiment the linker is a C6 amino alkyl group.

### Hepatitis B Virus

As used herein, “hepatitis B virus” or “HBV” refers to a member of the Hepadnaviridae family having a small double-stranded DNA genome of approximately 3,200 base pairs and a tropism for liver cells. “HBV” includes hepatitis B virus that infects any of a variety of mammalian (e.g., human, non-human primate, etc.) and avian (duck, etc.) hosts. “HBV” includes any known HBV genotype, e.g., serotype A, B, C, D, E, F, and G; any HBV serotype or HBV subtype; any HBV isolate; HBV variants, e.g., HBeAg-negative variants, drug-resistant HBV variants (e.g., lamivudine-resistant variants; adefovir-resistant mutants; tenofovir-resistant mutants; entecavir-resistant mutants; etc.); and the like.

“HBV” is a small DNA virus belonging to the Hepadnaviridae family and classified as the type species of the genus Orthohepadnavirus. HBV virus particles (virions) comprise an outer lipid envelope and an icosahedral nucleocapsid core composed of protein. The nucleocapsid generally encloses viral DNA and a DNA polymerase that has reverse transcriptase activity similar to retroviruses. The HBV outer envelope contains embedded proteins which are involved in viral binding of, and entry into, susceptible cells. HBV, which attacks the liver, has been classified according to at least ten genotypes (A-J) based on sequence. In general, there are four genes encoded by the genome, which genes are referred to as C, P, S, and X. The core protein is encoded by gene C (HBcAg), and its start codon is preceded by an upstream in-frame AUG start codon from which the pre-core protein is produced. HBeAg is produced by proteolytic processing of the pre-core protein. The DNA polymerase is encoded by gene P. Gene S encodes surface antigen (HBsAg). The HBsAg gene is one long open reading frame but contains three in frame “start” (ATG) codons that divide the gene into three sections, pre-S1, pre-S2, and S. Because of the multiple start codons, polypeptides of three different sizes called large, middle, and small (pre-S1+pre-S2+S, pre-S2+S, or S) are produced. These may have a ratio of 1:1:4 (Heermann et al, 1984).

Hepatitis B Virus (HBV) proteins can be organized into several categories and functions. Polymerases function as a reverse transcriptase (RT) to make viral DNA from pregenomic RNA (pgRNA), and also as a DNA-dependent polymerase to make covalently closed circular DNA (cccDNA) from viral DNA. They are covalently attached to the 5′ end of the minus strand. Core proteins make the viral capsid and the secreted E antigen. Surface antigens are the hepatocyte internalization ligands, and also the primary component of aviral spherical and filamentous particles. Aviral particles are produced >1000-fold over Dane particles (infectious virions) and may act as immune decoys.

### Hepatitis B Virus Surface Antigen

As used herein, the term “hepatitis B virus surface antigen” or “HBsAg” refers to an S-domain protein encoded by gene S (e.g., ORF S) of an HBV genome. Hepatitis B virus particles carry viral nucleic acid in core particles enveloped by three proteins encoded by gene S, which are the large surface, middle surface, and major surface proteins. Among these proteins, the major surface protein is generally about 226 amino acids and contains just the S-domain.

### Hepatitis B e Antigen (HBeAg)

As used herein, the term “Hepatitis B e antigen” or “HBeAg” is an indicator of viral replication, although some variant forms of the virus do not express HBeAg. Active infection can be described as HBeAg-positive or HBeAg-negative according to whether HBeAg is secreted.

### Infection

As used herein, the term “infection” refers to the pathogenic invasion and/or expansion of microorganisms, such as viruses, in a subject. An infection may be lysogenic, e.g., in which viral DNA lies dormant within a cell. Alternatively, an infection may be lytic, e.g., in which the virus actively proliferates and causes destruction of infected cells. An infection may or may not cause clinically apparent symptoms. An infection may remain localized, or it may spread, e.g., through a subject's blood or lymphatic system. An individual having, for example, an HBV infection can be identified by detecting one or more of viral load, surface antigen (HBsAg), e-antigen (HBeAg), and various other assays for detecting HBV infection known in the art. Assays for detection of HBV infection can involve testing serum or blood samples for the presence of HBsAg and/or HBeAg, and optionally further screening for the presence of one or more viral antibodies (e.g., IgM and/or IgG) to compensate for any periods in which an HBV antigen may be at an undetectable level.

### HBV Infection

The term “hepatitis B virus infection” or “HBV infection” is commonly known in the art and refers to an infectious disease that is caused by the hepatitis B virus (HBV) and affects the liver. A HBV infection can be an acute or a chronic infection. Some infected persons have no symptoms during the initial infection and some develop a rapid onset of sickness with vomiting, yellowish skin, tiredness, dark urine and abdominal pain (“Hepatitis B Fact sheet No 204”. who.int. July 2014. Retrieved 4 Nov. 2014). Often these symptoms last a few weeks and can result in death. It may take 30 to 180 days for symptoms to begin. In those who get infected around the time of birth 90% develop a chronic hepatitis B infection while less than 10% of those infected after the age of five do (“Hepatitis B FAQs for the Public-Transmission”, U.S. Centers for Disease Control and Prevention (CDC), retrieved 2011-11-29). Most of those with chronic disease have no symptoms; however, cirrhosis and liver cancer may eventually develop (Chang, 2007, Semin Fetal Neonatal Med, 12: 160-167). These complications result in the death of 15 to 25% of those with chronic disease (“Hepatitis B Fact sheet No 204”. who.int. July 2014, retrieved 4 Nov. 2014). Herein, the term “HBV infection” includes the acute and chronic hepatitis B infection. The term “HBV infection” also includes the asymptotic stage of the initial infection, the symptomatic stages, as well as the asymptotic chronic stage of the HBV infection.

### Liver Inflammation

As used herein, the term “liver inflammation” or “hepatitis” refers to a physical condition in which the liver becomes swollen, dysfunctional, and/or painful, especially as a result of injury or infection, as may be caused by exposure to a hepatotoxic agent. Symptoms may include jaundice (yellowing of the skin or eyes), fatigue, weakness, nausea, vomiting, appetite reduction, and weight loss. Liver inflammation, if left untreated, may progress to fibrosis, cirrhosis, liver failure, or liver cancer.

### Liver Fibrosis

As used herein, the term “liver fibrosis” or “fibrosis of the liver” refers to an excessive accumulation in the liver of extracellular matrix proteins, which could include collagens (I, III, and IV), fibronectin, undulin, elastin, laminin, hyaluronan, and proteoglycans resulting from inflammation and liver cell death. Liver fibrosis, if left untreated, may progress to cirrhosis, liver failure, or liver cancer.

### TLR7

As used herein, “TLR7” refers to the Toll-like receptor 7 of any species of origin (e.g., human, murine, woodchuck etc.).

### TLR7 Agonist

As used herein, “TLR7 agonist” refers to a compound that acts as an agonist of TLR7. Unless otherwise indicated, a TLR7 agonist can include the compound in any pharmaceutically acceptable form, including any isomer (e.g., diastereomer or enantiomer), salt, solvate, polymorph, and the like. The TLR agonism for a particular compound may be determined in any suitable manner. For example, assays for detecting TLR agonism of test compounds are described, for example, in U.S. Provisional Patent Application Ser. No. 60/432,650, filed Dec. 11, 2002, and recombinant cell lines suitable for use in such assays are described, for example, in U.S. Provisional Patent Application Ser. No. 60/432,651, filed Dec. 11, 2002. A further assay for evaluating TLR7 agonists is the HEK293-Blue-hTLR-7 cell assay described in Example 43 of WO2016/091698 (the assay is hereby incorporated by reference).

### Diastereomer

As used herein, the term “diastereomer” refers to a stereoisomer with two or more centers of chirality and whose molecules are not mirror images of one another. Diastereomers have different physical properties, e.g. melting points, boiling points, spectral properties, activities and reactivities.

Compounds of the general formulas (I)-(V) which contain one or several chiral centers can either be present as racemates, diastereomeric mixtures, or optically active single isomers. The racemates can be separated according to known methods into the enantiomers. Particularly, diastereomeric salts which can be separated by crystallization are formed from the racemic mixtures by reaction with an optically active acid such as e.g. D- or L-tartaric acid, mandelic acid, malic acid, lactic acid or camphorsulfonic acid.

### Pharmaceutically Acceptable Salts

The compounds according to the present invention may exist in the form of their pharmaceutically acceptable salts.

The term “pharmaceutically acceptable salts” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid, particularly hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein.

Alternatively, these salts may be prepared form addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins. The compound of formula (I) can also be present in the form of zwitterions. Particularly preferred pharmaceutically acceptable salts of compounds of formula (I) are the salts of hydrochloric acid, hydrobromic acid, sulfuric acid, phosphoric acid and methanesulfonic acid.

The chemical modification of a pharmaceutical compound into a salt is a technique well known to pharmaceutical chemists in order to obtain improved physical and chemical stability, hygroscopicity, flowability and solubility of compounds. It is for example described in Bastin, Organic Process Research & Development 2000, 4, 427-435 or in Ansel, In: Pharmaceutical Dosage Forms and Drug Delivery Systems, 6th ed. (1995), pp. 196 and 1456-1457. For example, the pharmaceutically acceptable salt of the compounds provided herein may be a sodium salt.

### Pharmaceutical Combination

As used herein a pharmaceutical combination is understood as the combination at least two different HBV therapeutics, e.g. active compounds or prodrugs (medical compounds or medicaments), for treatment of a disease. A pharmaceutical combination can involve compounds that are physically, chemically, or otherwise combined (e.g., in the same vial); compounds that are packaged together (e.g., as two separate objects in the same package (kit of parts) either for simultaneous administration or separate administration); or compounds that are provided separately but intended to be used together (e.g. the combination is expressly stated on the compound label, instructions or package insert). In one embodiment the pharmaceutical combination consists of a medical compound formulated for oral administration and a medical compound formulated for subcutaneous injection.

### Approximately

As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

### Administering

As used herein, the terms “administering” or “administration” means to provide a substance (e.g., a pharmaceutical combination or an oligonucleotide) to a subject in a manner that is pharmacologically useful (e.g., to treat a condition in the subject).

### Asialoglycoprotein Receptor (ASGPR)

As used herein, the term “Asialoglycoprotein receptor” or “ASGPR” refers to a bipartite C-type lectin formed by a major 48 kDa (ASGPR-1) and minor 40 kDa subunit (ASGPR-2). ASGPR is primarily expressed on the sinusoidal surface of hepatocyte cells and has a major role in binding, internalization, and subsequent clearance of circulating glycoproteins that contain terminal galactose or N-acetylgalactosamine residues (asialoglycoproteins).

### Prodrug

As used herein, the term “prodrug” refers to a form or derivative of a compound which is metabolized in vivo, e.g., by biological fluids or enzymes by a subject after administration, into a pharmacologically active form of the compound in order to produce the desired pharmacological effect. Prodrugs are described e.g. in the Organic Chemistry of Drug Design and Drug Action by Richard B. Silverman, Academic Press, San Diego, 2004, Chapter 8 Prodrugs and Drug Delivery Systems, pp. 497-558.

### Subject

As used herein, the term “subject” means any mammal, including mice, rabbits, and humans. In one embodiment, the subject is a human or non-human primate. The terms “individual” or “patient” may be used interchangeably with “subject.”

### Treatment

The terms “treatment”, “treating”, “treats” or the like are used herein generally mean obtaining a desired pharmacological and/or physiological effect. This effect is therapeutic in terms of partially or completely curing a disease and/or adverse effect attributed to the disease. The effect is provided through the administration a therapeutic agent (e.g., a pharmaceutical combination or an oligonucleotide) to the subject, for purposes of improving the health and/or well-being of the subject with respect to an existing condition (e.g., an existing HBV infection) or to prevent or decrease the likelihood of the occurrence of a condition (e.g., preventing liver fibrosis, hepatitis, liver cancer or other condition associated with an HBV infection). The term “treatment” as used herein covers any treatment of HBV infection in a subject and includes: (a) inhibiting the disease, i.e. arresting its development like the inhibiting of increase of HBsAg and/or HBeAg; or (b) ameliorating (i.e. relieving) the disease, i.e. causing regression of the disease, like the repression of HBsAg and/or HBeAg production. Thus, a compound or compound combination that ameliorates and/or inhibits a HBV infection is a compound or compound combination that treats a HBV invention. Preferably, the term “treatment” as used herein relates to medical intervention of an already manifested disorder, like the treatment of an already defined and manifested HBV infection, in particular a chronic HBV infection. In some embodiments, treatment involves reducing the frequency or severity of at least one sign, symptom or contributing factor of a condition (e.g., HBV infection or related condition) experienced by a subject. During an HBV infection, a subject may exhibit symptoms such as yellowing of the skin and eyes (jaundice), dark urine, extreme fatigue, nausea, vomiting and abdominal pain. Accordingly, in some embodiments, a treatment, e.g. a pharmaceutical combination, provided herein may result in a reduction in the frequency or severity of one or more of such symptoms. However, HBV infection can develop into one or more liver conditions, such as cirrhosis, liver fibrosis, liver inflammation or liver cancer. Accordingly, in some embodiments, a treatment, e.g. pharmaceutical combination, provided herein may result in a reduction in the frequency or severity of, or prevent or attenuate, one or more of such conditions.

### Therapeutic Effective Amount

The term “therapeutically effective amount” denotes an amount of a compound the pharmaceutical combination of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the compound, the disease state being treated, the severity of the disease treated, the age and relative health of the subject, the route and form of administration, the judgement of the attending medical or veterinary practitioner, and other factors.

### Excipient

As used herein, the term “excipient” refers to a non-therapeutic agent that may be included in one or more of the compositions comprising a medicament which is part of a pharmaceutical combination, for example, to provide or contribute to a desired consistency or stabilizing effect.

### DETAILED DESCRIPTION OF THE INVENTION

The present invention relates to pharmaceutical combinations comprising at least two HBV therapeutics. More particularly, the present invention relates to a pharmaceutical combination comprising an RNAi oligonucleotide targeting HBV and an anti-PDL1 antisense oligonucleotide as defined herein.

The HBV therapeutics and dosage regimes to be used in the pharmaceutical combinations of the present invention will now be described in detail.

### RNAi Oligonucleotide Targeting HBV

In an embodiment, a therapeutic used in the pharmaceutical combination of the present invention is an RNAi oligonucleotide targeting HBV that can be used to achieve a therapeutic benefit. This RNAi oligonucleotide is capable of reducing the expression of HBsAg mRNA. In an embodiment, the RNAi oligonucleotide in the pharmaceutical combination of the present invention is an oligonucleotide targeting HBsAg mRNA.

In an embodiment, the RNAi oligonucleotide in the pharmaceutical combination of the present invention is an oligonucleotide which targets HBsAg mRNA, thereby reducing the expression of HBsAg mRNA.

Through examination of HBV surface antigen mRNA and testing of different oligonucleotides, potent oligonucleotides have been developed for reducing expression of HBV surface antigen (HBsAg) to treat HBV infection. RNAi oligonucleotides provided herein, in some embodiments, are designed to target HBsAg mRNA sequences covering >95% of known HBV genomes across all known genotypes. In some embodiments, such oligonucleotides when used as part of a pharmaceutical combination of the invention result in more than 90% reduction of HBV pre-genomic RNA (pgRNA) and HBsAg mRNAs in liver. In some embodiments, the reduction in HBsAg expression persists for an extended period of time following a treatment regimen of the pharmaceutical combination.

Accordingly, in some embodiments, RNAi oligonucleotides provided herein are designed so as to have regions of complementarity to HBsAg mRNA for purposes of targeting the transcripts in cells and inhibiting their expression. The region of complementarity is generally of a suitable length and base content to enable annealing of the oligonucleotide (or a strand thereof) to HBsAg mRNA for purposes of inhibiting its expression. In some embodiments, the region of complementarity is at least 12, at least 13, at least 14, at least 15, at least 16, at least 17, at least 18, at least 19 or at least 20 nucleotides in length. In some embodiments, an oligonucleotide provided herein has a region of complementarity to HBsAg mRNA that is in the range of 12 to 30 (e.g., 12 to 30, 12 to 22, 15 to 25, 17 to 21, 18 to 27, 19 to 27, or 15 to 30) nucleotides in length. In some embodiments, an RNAi oligonucleotide provided herein has a region of complementarity to HBsAg mRNA that is 12, 13, 14, 15, 16, 17, 18, 19, 20, 21, 22, 23, 24, 25, 26, 27, 28, 29 or 30 nucleotides in length.

In some embodiments, RNAi oligonucleotides provided herein are designed to target mRNA sequences encoding HBsAg. For example, in some embodiments, an RNAi oligonucleotide is provided that has an antisense strand having a region of complementarity to a sequence set forth as: ACAANAAUCCUCACAAUA (SEQ ID NO: 1), which N refers to any nucleotide (A, G, T/U, or C). In some embodiments, the oligonucleotide further comprises a sense strand that forms a duplex region with the antisense strand. In some embodiments, the sense strand has a region of complementarity to a sequence set forth as: UUNUUGUGAGGAUUN (SEQ ID NO: 2). In some embodiments, the sense strand comprises a region of complementarity to a sequence as set forth in (shown 5′ to 3′): UUAUUGUGAGGAUUNUUGUC (SEQ ID NO: 3).

In some embodiments, the antisense strand comprises, or consists of, a sequence set forth as: UUAUUGUGAGGAUUNUUGUCGG (SEQ ID NO: 4). In some embodiments, the antisense strand comprises, or consists of, a sequence set forth as: UUAUUGUGAGGAUUCUUGUCGG (SEQ ID NO: 5). In some embodiments, the antisense strand comprises, or consists of, a sequence set forth as: UUAUUGUGAGGAUUUUUGUCGG (SEQ ID NO: 6). In some embodiments, the sense strand comprises, or consists of, a sequence set forth as: ACAANAAUCCUCACAAUAA (SEQ ID NO: 7). In some embodiments, the sense strand comprises, or consists of, a sequence set forth as: GACAANAAUCCUCACAAUAAGCAGCCGAAAGGCUGC (SEQ ID NO: 8). In some embodiments, the sense strand comprises, or consists of, a sequence set forth as: GACAAAAAUCCUCACAAUAAGCAGCCGAAAGGCUGC (SEQ ID NO: 9). In some embodiments, the sense strand comprises, or consists of, a sequence set forth as: GACAAGAAUCCUCACAAUAAGCAGCCGAAAGGCUGC (SEQ ID NO: 10).

In some embodiments, the RNAi oligonucleotide for reducing expression of HBsAg mRNA comprises a sense strand forming a duplex region with an antisense strand, where the sense strand comprises a sequence as set forth in any one of SEQ ID NOs: 7-10, and the antisense strand comprises a sequence as set forth in any one of SEQ ID NOs: 4-6. In some embodiments, the sense strand comprises 2′-fluoro and 2′-O-methyl modified nucleotides and at least one phosphorothioate internucleotide linkage. In some embodiments, the sense strand is conjugated to an N-acetylgalactosamine (GalNAc) moiety. In some embodiments, the antisense strand comprises 2′-fluoro and 2′-O-methyl modified nucleotides and at least one phosphorothioate internucleotide linkage. In some embodiments, the 4′-carbon of the sugar of the 5′-nucleotide of the antisense strand comprises a phosphate analog. In some embodiments, each of the antisense strand and the sense strand comprises 2′-fluoro and 2′-O-methyl modified nucleotides and at least one phosphorothioate internucleotide linkage, where the 4′-carbon of the sugar of the 5′-nucleotide of the antisense strand comprises a phosphate analog, and the sense strand is conjugated to an N-acetylgalactosamine (GalNAc) moiety. In some embodiments, a sense strand comprising a sequence as set forth in any one of SEQ ID NOs: 8-10 comprises 2′-fluoro modified nucleotides at positions 3, 8-10, 12, 13, and 17. In some embodiments, the sense strand comprises 2′-O-methyl modified nucleotides at positions 1, 2, 4-7, 11, 14-16, 18-26, and 31-36. In some embodiments, the sense strand comprises one phosphorothioate internucleotide linkage. In some embodiments, the sense strand comprises a phosphorothioate internucleotide linkage between nucleotides at positions 1 and 2. In some embodiments, the sense strand is conjugated to an N-acetylgalactosamine (GalNAc) moiety.

In some embodiments, an antisense strand comprising a sequence as set forth in any one of SEQ ID NOs: 4-6 comprises 2′-fluoro modified nucleotides at positions 2, 3, 5, 7, 8, 10, 12, 14, 16, and 19. In some embodiments, the antisense strand comprises 2′-O-methyl modified nucleotides at positions 1, 4, 6, 9, 11, 13, 15, 17, 18, and 20-22. In some embodiments, the antisense strand comprises three phosphorothioate internucleotide linkages. In some embodiments, the antisense strand comprises phosphorothioate internucleotide linkages between nucleotides at positions 1 and 2, between nucleotides at positions 2 and 3, between nucleotides at positions 3 and 4, between nucleotides at positions 20 and 21, and between nucleotides at positions 21 and 22. In some embodiments, the 4′-carbon of the sugar of the 5′-nucleotide of the antisense strand comprises a phosphate analog.

In an embodiment in the pharmaceutical combination of the present invention the RNAi oligonucleotide is an oligonucleotide comprising an antisense strand of 19 to 30 nucleotides in length, wherein the antisense strand comprises a region of complementarity to a sequence of HBsAg mRNA set forth as ACAANAAUCCUCACAAUA (SEQ ID NO: 1) (N can refer to any nucleotide A, G, C or T/U). In some embodiments, the oligonucleotide further comprises a sense strand that forms a duplex region with the antisense strand. In some embodiments, the sense strand has a region of complementarity to the sequence set forth as UUNUUGUGAGGAUUN (SEQ ID NO: 2). In some embodiments, the sense strand comprises a region of complementarity to a sequence as set forth as (shown 5′ to 3′) UUAUUGUGAGGAUUNUUGUC (SEQ ID NO: 3).

In an embodiment, the RNAi oligonucleotide in the pharmaceutical combination of the present invention is an oligonucleotide for reducing expression of hepatitis B virus surface antigen (HBsAg) mRNA, the oligonucleotide comprising a sense strand forming a duplex region with an antisense strand, wherein:
the sense strand consists of a sequence GACAAAAAUCCUCACAAUAAGCAGCCGAAAGGCUGC (SEQ ID NO: 9) and comprising 2′-fluoro modified nucleotides at positions 3, 8-10, 12, 13, and 17, 2′-O-methyl modified nucleotides at positions 1, 2, 4-7, 11, 14-16, 18-26, and 31-36, and a phosphorothioate linkage between the nucleotides at positions 1 and 2, wherein each of the nucleotides of the -GAAA-sequence on the sense strand is conjugated to a monovalent GalNac moiety; and
the antisense strand consists of a sequence UUAUUGUGAGGAUUUUUGUCGG (SEQ ID NO: 6) and comprising 2′-fluoro modified nucleotides at positions 2, 3, 5, 7, 8, 10, 12, 14, 16, and 19, 2′-O-methyl modified nucleotides at positions 1, 4, 6, 9, 11, 13, 15, 17, 18, and 20-22, and phosphorothioate linkages between nucleotides at positions 1 and 2, between nucleotides at positions 2 and 3, between nucleotides at positions 3 and 4, between nucleotides at positions 20 and 21, and between nucleotides at positions 21 and 22,
wherein the 4′-carbon of the sugar of the 5′-nucleotide of the antisense strand comprises a methoxy phosphonate (MOP).

In a preferred embodiment, the RNAi oligonucleotide in the pharmaceutical combination of the present invention is an oligonucleotide comprising a sense strand forming a duplex region with an antisense strand, wherein:
the sense strand comprises a sequence GACAAAAAUCCUCACAAUAAGCAGCCGAAAGGCUGC (SEQ ID NO: 9) and comprising 2′-fluoro modified nucleotides at positions 3, 8-10, 12, 13, and 17, 2′-O-methyl modified nucleotides at positions 1, 2, 4-7, 11, 14-16, 18-26, and 31-36, and one phosphorothioate internucleotide linkage between the nucleotides at positions 1 and 2, wherein each of the nucleotides of the -GAAA- sequence on the sense strand is conjugated to a monovalent GalNac moiety, wherein the -GAAA- sequence comprises the structure:

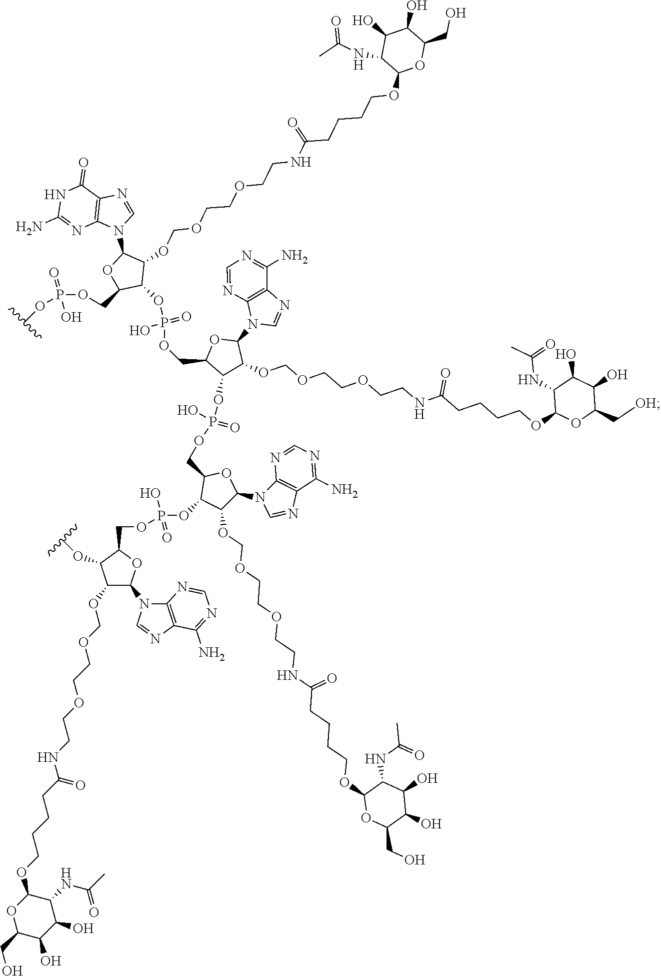 [SMILES: *O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(=O)[nH]c(N)nc32)O[C@@H]1COP(=O)(O)O*]

and the antisense strand comprises a sequence as set forth in UUAUUGUGAGGAUUUUUGUCGG (SEQ ID NO: 6) and comprising 2′-fluoro modified nucleotides at positions 2, 3, 5, 7, 8, 10, 12, 14, 16, and 19, 2′-O-methyl modified nucleotides at positions 1, 4, 6, 9, 11, 13, 15, 17, 18, and 20-22, and five phosphorothioate internucleotide linkages between nucleotides 1 and 2, 2 and 3, 3 and 4, 20 and 21, and 21 and 22, wherein the 5′-nucleotide of the antisense strand has the following structure:

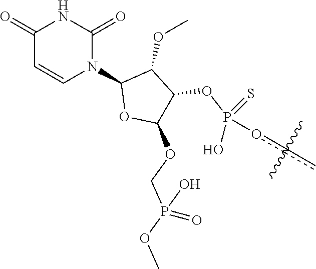

or a pharmaceutically acceptable salt thereof. This definition of the RNAi oligonucleotide targeting HBV used in the pharmaceutical combinations of the present invention is referred to herein as “T1” or “Therapeutic T1”.

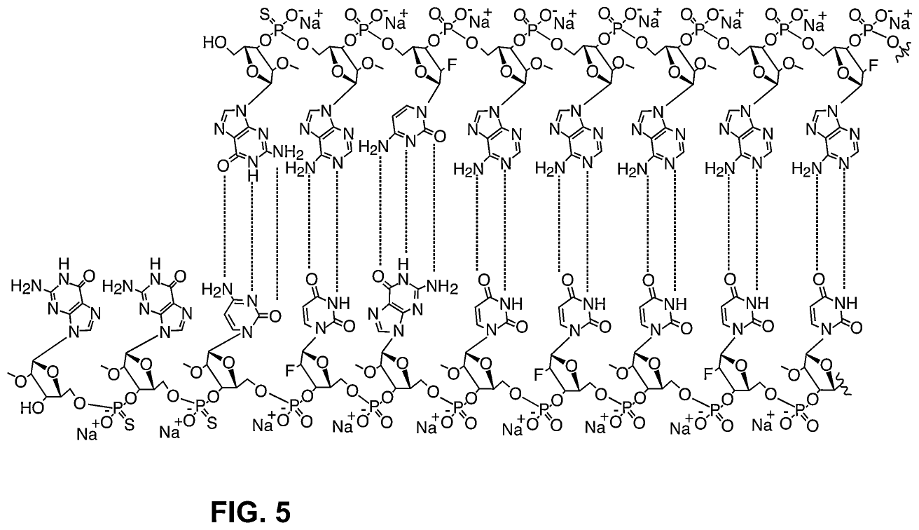

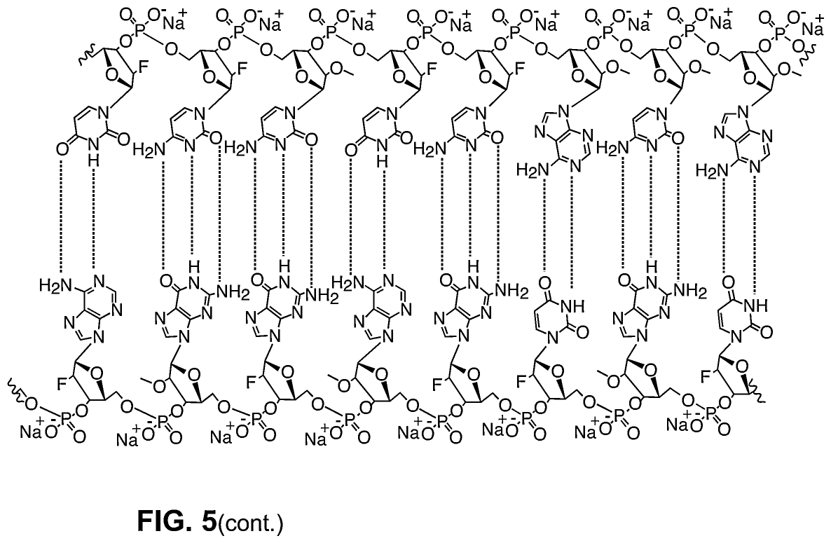

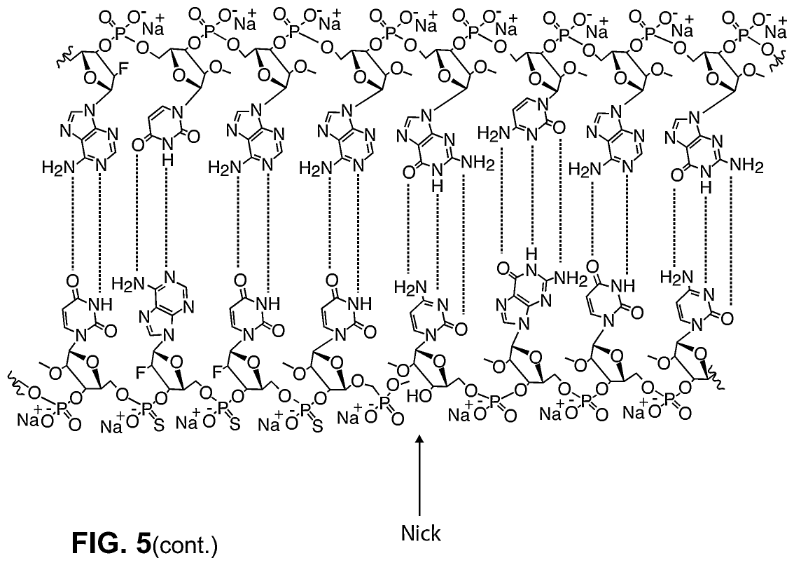

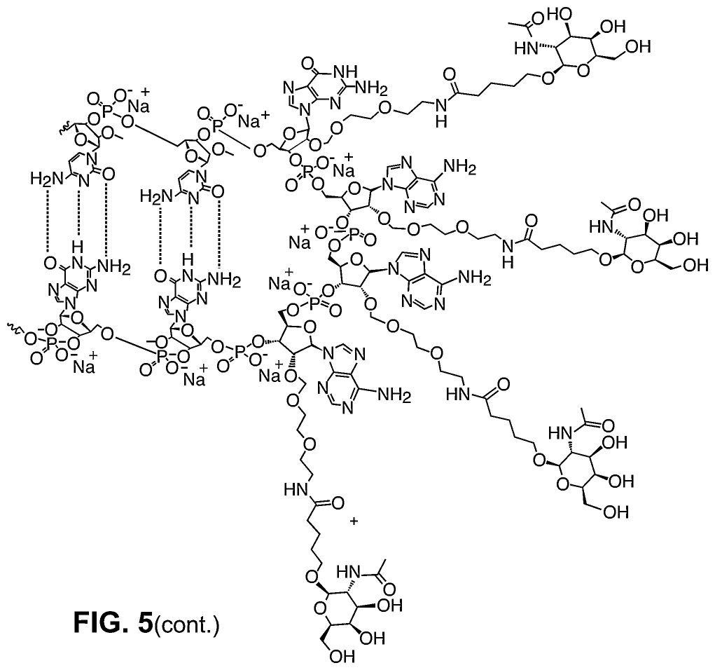

In a particular specific embodiment, T1 may be further defined as the molecule in FIG. 5.

In an embodiment, the RNAi oligonucleotide is administered subcutaneously.

In an embodiment, the RNAi oligonucleotide is administered at an initial dose of about 0.1 mg/kg to about 12 mg/kg, preferably of about 0.1 mg/kg to about 9 mg/kg, more preferably of about 0.5 mg/kg to about 7 mg/kg, more preferably of about 0.5 mg/kg to about 6.5 mg/kg, more preferably of about 1 mg/kg to about 6 mg/kg, more preferably of about 1.5 mg/kg to about 6 mg/kg, more preferably of about 2 mg/kg to about 6 mg/kg, most preferably of about 3 mg/kg or about 6 mg/kg.

In an embodiment, the RNAi oligonucleotide is administered at an initial dose of from about 6 to about 800 mg, preferably about 100 mg, about 200 mg or about 400 mg.

In an embodiment, the initial dose is a single dose or is the only dose administered.

In an embodiment one or more subsequent doses of the RNAi oligonucleotide in an amount that is from about 0.1 mg/kg to about 12 mg/kg are administered. In an embodiment, the subsequent dose(s) is about 1.5 mg/kg, about 3 mg/kg or about 6 mg/kg.

In an embodiment, one or more subsequent doses of the oligonucleotide in an amount that is from about 6 mg to about 800 mg is administered. In an embodiment, the subsequent dose(s) is about 100 mg, about 200 mg or about 400 mg.

In an embodiment, each dose is administered at least about once every two weeks, at least about once every three weeks, at least about once every four weeks, at least about once every five weeks, at least about once every six weeks, at least about once every seven weeks, or at least about once every eight weeks. In an embodiment, the doses are separated in time from each other by at least about four weeks. In an embodiment, doses of about 1 mg/kg to 6 mg/kg are administered, each separated by at least about four weeks.

In an embodiment, the doses are separated in time from each other by about four weeks and are administered over a period of about 48 weeks, about 24 weeks, about three months or about 12 weeks.

In an embodiment, the period of time between each of the doses is independently selected from the group consisting of: about four weeks, about one month, about two months, about three months or about six months.

Further useful definitions and permutations of the RNAi oligonucleotide targeting HBV in the pharmaceutical combinations of the present invention are provided below.

I. Double-Stranded Oligonucleotides for Targeting HBsAg mRNA

There are a variety of structures of oligonucleotides that are useful for targeting HBsAg mRNA expression in the pharmaceutical combinations of the present disclosure, including RNAi, miRNA, etc. Any of the structures described herein or elsewhere may be used as a framework to incorporate or target a sequence described herein. Double-stranded oligonucleotides for targeting HBV antigen expression (e.g., via the RNAi pathway) generally have a sense strand and an antisense strand that form a duplex with one another. In some embodiments, the sense and antisense strands are not covalently linked. However, in some embodiments, the sense and antisense strands are covalently linked.

In some embodiments of the present invention, double-stranded oligonucleotides for reducing the expression of HBsAg mRNA expression engage RNA interference (RNAi). For example, RNAi oligonucleotides have been developed with each strand having sizes of 19-25 nucleotides with at least one 3′ overhang of 1 to 5 nucleotides (see, e.g., U.S. Pat. No. 8,372,968). Longer oligonucleotides have also been developed that are processed by Dicer to generate active RNAi products (see, e.g., U.S. Pat. No. 8,883,996). Further work produced extended double-stranded oligonucleotides where at least one end of at least one strand is extended beyond a duplex targeting region, including structures where one of the strands includes a thermodynamically-stabilizing tetraloop structure (see, e.g., U.S. Pat. Nos. 8,513,207 and 8,927,705, as well as WO2010033225, which are incorporated by reference herein for their disclosure of these oligonucleotides). Such structures may include single-stranded extensions (on one or both sides of the molecule) as well as double-stranded extensions.

In some embodiments, oligonucleotides provided herein are cleavable by Dicer enzymes. Such oligonucleotides may have an overhang (e.g., of 1, 2, or 3 nucleotides in length) in the 3′ end of the sense strand. Such oligonucleotides (e.g., siRNAs) may comprise a 21 nucleotide guide strand that is antisense to a target RNA and a complementary passenger strand, in which both strands anneal to form a 19-bp duplex and 2 nucleotide overhangs at either or both 3′ ends. Longer oligonucleotide designs are also available including oligonucleotides having a guide strand of 23 nucleotides and a passenger strand of 21 nucleotides, where there is a blunt end on the right side of the molecule (3′-end of passenger strand/5′-end of guide strand) and a two nucleotide 3′-guide strand overhang on the left side of the molecule (5′-end of the passenger strand/3′-end of the guide strand). In such molecules, there is a 21 base pair duplex region. See, for example, U.S. Pat. Nos. 9,012,138, 9,012,621, and 9,193,753, each of which are incorporated herein for their relevant disclosures.

In some embodiments, oligonucleotides as disclosed herein may comprise sense and antisense strands that are both in the range of 17 to 26 (e.g., 17 to 26, 20 to 25, 19 to 21 or 21-23) nucleotides in length. In some embodiments, the sense and antisense strands are of equal length. In some embodiments, for oligonucleotides that have sense and antisense strands that are both in the range of 21-23 nucleotides in length, a 3′ overhang on the sense, antisense, or both sense and antisense strands is 1 or 2 nucleotides in length. In some embodiments, the oligonucleotide has a guide strand of 23 nucleotides and a passenger strand of 21 nucleotides, where there is a blunt end on the right side of the molecule (3′-end of passenger strand/5′-end of guide strand) and a two nucleotide 3′-guide strand overhang on the left side of the molecule (5′-end of the passenger strand/3′-end of the guide strand). In such molecules, there is a 21 base pair duplex region. In some embodiments, an oligonucleotide comprises a 25 nucleotide sense strand and a 27 nucleotide antisense strand that when acted upon by a dicer enzyme results in an antisense strand that is incorporated into the mature RISC.

Other oligonucleotide designs for use with the compositions and methods disclosed herein include: 16-mer siRNAs (see, e.g., Nucleic Acids in Chemistry and Biology. Blackburn (ed.), Royal Society of Chemistry, 2006), shRNAs (e.g., having 19 bp or shorter stems; see, e.g., Moore et al. Methods Mol. Biol. 2010; 629:141-158), blunt siRNAs (e.g., of 19 bps in length; see: e.g., Kraynack and Baker, RNA Vol. 12, p 163-176 (2006)), asymmetrical siRNAs (aiRNA; see, e.g., Sun et al., Nat. Biotechnol. 26, 1379-1382 (2008)), asymmetric shorter-duplex siRNA (see, e.g., Chang et al., Mol Ther. 2009 April; 17(4): 725-32), fork siRNAs (see, e.g., Hohjoh, FEBS Letters, Vol 557, issues 1-3; January 2004, p 193-198), single-stranded siRNAs (Eisner; Nature Biotechnology 30, 1063 (2012)), dumbbell-shaped circular siRNAs (see, e.g., Abe et al. J Am Chem Soc 129: 15108-15109 (2007)), and small internally segmented interfering RNA (sisiRNA; see, e.g., Bramsen et al., Nucleic Acids Res. 2007 September; 35(17): 5886-5897). Each of the foregoing references is incorporated by reference in its entirety for the related disclosures therein. Further non-limiting examples of oligonucleotide structures that may be used in some embodiments in a pharmaceutical combination to reduce or inhibit the expression of HBsAg are microRNA (miRNA), short hairpin RNA (shRNA), and short siRNA (see, e.g., Hamilton et al., Embo J., 2002, 21(17): 4671-4679; see also U.S. Application No. 20090099115).

a. Antisense Strands

In some embodiments, an antisense strand of an oligonucleotide may be referred to as a “guide strand”. For example, if an antisense strand can engage with RNA-induced silencing complex (RISC) and bind to an Argonaut protein, or engage with or bind to one or more similar factors, and direct silencing of a target gene, it may be referred to as a guide strand. In some embodiments, a sense strand complementary with a guide strand may be referred to as a “passenger strand”.

In some embodiments, an oligonucleotide provided herein comprises an antisense strand that is up to 50 nucleotides in length (e.g., up to 30, up to 27, up to 25, up to 21, or up to 19 nucleotides in length). In some embodiments, an oligonucleotide provided herein comprises an antisense strand that is at least 12 nucleotides in length (e.g., at least 12, at least 15, at least 19, at least 21, at least 25, or at least 27 nucleotides in length). In some embodiments, an antisense strand of an oligonucleotide disclosed herein is in the range of 12 to 50 or 12 to 30 (e.g., 12 to 30, 11 to 27, 11 to 25, 15 to 21, 15 to 27, 17 to 21, 17 to 25, 19 to 27, or 19 to 30) nucleotides in length. In some embodiments, an antisense strand of any one of the oligonucleotides disclosed herein is 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length.

b. Sense Strands

In some embodiments, a double-stranded oligonucleotide may have a sense strand of up to 40 nucleotides in length (e.g., up to 40, up to 35, up to 30, up to 27, up to 25, up to 21, up to 19, up to 17, or up to 12 nucleotides in length). In some embodiments, an oligonucleotide may have a sense strand of at least 12 nucleotides in length (e.g., at least 12, at least 15, at least 19, at least 21, at least 25, at least 27, at least 30, at least 35, or at least 38 nucleotides in length). In some embodiments, an oligonucleotide may have a sense strand in a range of 12 to 50 (e.g., 12 to 40, 12 to 36, 12 to 32, 12 to 28, 15 to 40, 15 to 36, 15 to 32, 15 to 28, 17 to 21, 17 to 25, 19 to 27, 19 to 30, 20 to 40, 22 to 40, 25 to 40, or 32 to 40) nucleotides in length. In some embodiments, an oligonucleotide may have a sense strand of 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in length. In some embodiments, a sense strand of an oligonucleotide is longer than 27 nucleotides (e.g., 28, 29, 30, 31, 32, 33, 34, 35, 36, 37, 38, 39 or 40 nucleotides). In some embodiments, a sense strand of an oligonucleotide is longer than 25 nucleotides (e.g., 26, 27, 28, 29 or 30 nucleotides).

In some embodiments, a sense strand comprises a stem-loop at its 3′-end. In some embodiments, a sense strand comprises a stem-loop at its 5′-end. In some embodiments, a strand comprising a stem loop is in the range of 2 to 66 nucleotides long (e.g., 2 to 66, 10 to 52, 14 to 40, 2 to 30, 4 to 26, 8 to 22, 12 to 18, 10 to 22, 14 to 26, or 14 to 30 nucleotides long). In some embodiments, a strand comprising a stem loop is 8, 9, 10, 11, 12, 13, 14, 15, 16, 17, 18, 19, 20, 21, 22, 23, 24, 25, 26, 27, 28, 29, or 30 nucleotides in length. In some embodiments, a stem comprises a duplex of 1, 2, 3, 4, 5, 6, 7, 8, 9, 10, 11, 12, 13, or 14 nucleotides in length. In some embodiments, a stem-loop provides the molecule better protection against degradation (e.g., enzymatic degradation) and facilitates targeting characteristics for delivery to a target cell. For example, in some embodiments, a loop provides added nucleotides on which modification can be made without substantially affecting the gene expression inhibition activity of an oligonucleotide. In certain embodiments, an oligonucleotide is provided herein in which the sense strand comprises (e.g., at its 3′-end) a stem-loop set forth as: S₁-L-S₂, in which S₁ is complementary to S₂, and in which L forms a loop between S1 and S₂ of up to 10 nucleotides in length (e.g., 1, 2, 3, 4, 5, 6, 7, 8, 9, or 10 nucleotides in length).

In some embodiments, a loop (L) of a stem-loop is a tetraloop (e.g., within a nicked tetraloop structure). A tetraloop may contain ribonucleotides, deoxyribonucleotides, modified nucleotides, and combinations thereof. Typically, a tetraloop has 4 to 5 nucleotides.

c. Duplex Length

In some embodiments, a duplex formed between a sense and antisense strand is at least 12 (e.g., at least 15, at least 16, at least 17, at least 18, at least 19, at least 20, or at least 21) nucleotides in length. In some embodiments, a duplex formed between a sense and antisense strand is in the range of 12-30 nucleotides in length (e.g., 12 to 30, 12 to 27, 12 to 22, 15 to 25, 18 to 30, 18 to 22, 18 to 25, 18 to 27, 18 to 30, 19 to 30 or 21 to 30 nucleotides in length). In some embodiments, a duplex formed between a sense and antisense strand is 12, 13, 14, 15, 16, 17, 18, 19, 20, 21, 22, 23, 24, 25, 26, 27, 28, 29, or 30 nucleotides in length. In some embodiments a duplex formed between a sense and antisense strand does not span the entire length of the sense strand and/or antisense strand. In some embodiments, a duplex between a sense and antisense strand spans the entire length of either the sense or antisense strands. In certain embodiments, a duplex between a sense and antisense strand spans the entire length of both the sense strand and the antisense strand.

d. Oligonucleotide Ends

In some embodiments, an oligonucleotide comprises sense and antisense strands, such that there is a 3′-overhang on either the sense strand or the antisense strand, or both the sense and antisense strand. In some embodiments, oligonucleotides provided herein have one 5′ end that is thermodynamically less stable compared to the other 5′ end. In some embodiments, an asymmetry oligonucleotide is provided that includes a blunt end at the 3′ end of a sense strand and an overhang at the 3′ end of an antisense strand. In some embodiments, a 3′ overhang on an antisense strand is 1-8 nucleotides in length (e.g., 1, 2, 3, 4, 5, 6, 7 or 8 nucleotides in length).

Typically, an oligonucleotide for RNAi has a two nucleotide overhang on the 3′ end of the antisense (guide) strand. However, other overhangs are possible. In some embodiments, an overhang is a 3′ overhang comprising a length of between one and six nucleotides, optionally one to five, one to four, one to three, one to two, two to six, two to five, two to four, two to three, three to six, three to five, three to four, four to six, four to five, five to six nucleotides, or one, two, three, four, five or six nucleotides. However, in some embodiments, the overhang is a 5′ overhang comprising a length of between one and six nucleotides, optionally one to five, one to four, one to three, one to two, two to six, two to five, two to four, two to three, three to six, three to five, three to four, four to six, four to five, five to six nucleotides, or one, two, three, four, five or six nucleotides.

In some embodiments, one or more (e.g., 2, 3, 4) terminal nucleotides of the 3′ end or 5′ end of a sense and/or antisense strand are modified. For example, in some embodiments, one or two terminal nucleotides of the 3′ end of an antisense strand are modified. In some embodiments, the last nucleotide at the 3′ end of an antisense strand is modified, e.g., comprises 2′-modification, e.g., a 2′-O-methoxyethyl. In some embodiments, the last one or two terminal nucleotides at the 3′ end of an antisense strand are complementary with the target. In some embodiments, the last one or two nucleotides at the 3′ end of the antisense strand are not complementary with the target.

In some embodiments, a double stranded oligonucleotide is provided that has a nicked tetraloop structure at the 3′ end sense strand, and two terminal overhang nucleotides at the 3′ end of its antisense strand. In some embodiments, the two terminal overhang nucleotides are GG. Typically, one or both of the two terminal GG nucleotides of the antisense strand is or are not complementary with the target.

In some embodiments, the 5′ end and/or the 3′ end of a sense or antisense strand has an inverted cap nucleotide.

In some embodiments, one or more (e.g., 2, 3, 4, 5, 6) modified internucleotide linkages are provided between terminal nucleotides of the 3′ end or 5′ end of a sense and/or antisense strand. In some embodiments, modified internucleotide linkages are provided between overhang nucleotides at the 3′ end or 5′ end of a sense and/or antisense strand.

e. Mismatches

In some embodiments, an oligonucleotide may have one or more (e.g., 1, 2, 3, 4, 5) mismatches between a sense and antisense strand. If there is more than one mismatch between a sense and antisense strand, they may be positioned consecutively (e.g., 2, 3 or more in a row), or interspersed throughout the region of complementarity. In some embodiments, the 3′-terminus of the sense strand contains one or more mismatches. In one embodiment, two mismatches are incorporated at the 3′ terminus of the sense strand. In some embodiments, base mismatches or destabilization of segments at the 3′-end of the sense strand of the oligonucleotide improved the potency of synthetic duplexes in RNAi, possibly through facilitating processing by Dicer.

In some embodiments, an antisense strand may have a region of complementarity to an HBsAg transcript that contains one or more mismatches compared with a corresponding transcript sequence. A region of complementarity on an oligonucleotide may have up to 1, up to 2, up to 3, up to 4, up to 5, etc. mismatches provided that it maintains the ability to form complementary base pairs with the transcript under appropriate hybridization conditions. Alternatively, a region of complementarity of an oligonucleotide may have no more than 1, no more than 2, no more than 3, no more than 4, or no more than 5 mismatches provided that it maintains the ability to form complementary base pairs with HBsAg mRNA under appropriate hybridization conditions. In some embodiments, if there are more than one mismatches in a region of complementarity, they may be positioned consecutively (e.g., 2, 3, 4, or more in a row), or interspersed throughout the region of complementarity provided that the oligonucleotide maintains the ability to form complementary base pairs with HBsAg mRNA under appropriate hybridization conditions.

### II. Single-Stranded Oligonucleotides

In some embodiments, an RNAi oligonucleotide for reducing HBsAg expression as described herein is a single-stranded oligonucleotide having complementarity with HBsAg mRNA. Such structures may include, but are not limited to single-stranded RNAi oligonucleotides. Recent efforts have demonstrated the activity of single-stranded RNAi oligonucleotides (see, e.g., Matsui et al. (May 2016), Molecular Therapy, Vol. 24(5), 946-955).

While such a single-stranded RNAi oligonucleotide may technically be considered an antisense oligonucleotide, it can still function through the mechanism of RNA interference and will have the characteristics as described herein for an RNAi oligonucleotide.

III. Oligonucleotide modifications

The modifications discussed in this section are especially preferable for implementation in the RNAi oligonucleotide of the present invention.

Oligonucleotides may be modified in various ways to improve or control specificity, stability, delivery, bioavailability, resistance from nuclease degradation, immunogenicity, base-paring properties, RNA distribution and cellular uptake and other features relevant to therapeutic or research use. See, e.g., Bramsen et al., Nucleic Acids Res., 2009, 37, 2867-2881; Bramsen and Kjems (Frontiers in Genetics, 3 (2012): 1-22). Accordingly, in some embodiments, therapeutic oligonucleotides of the present disclosure may include one or more suitable modifications. In some embodiments, a modified nucleotide has a modification in its base (or nucleobase), the sugar (e.g., ribose, deoxyribose), or the phosphate group.

The number of modifications on an oligonucleotide and the positions of those nucleotide modifications may influence the properties of an oligonucleotide. For example, oligonucleotides may be delivered in vivo by conjugating them to or encompassing them in a lipid nanoparticle (LNP) or similar carrier. However, when an oligonucleotide is not protected by an LNP or similar carrier, it may be advantageous for at least some of its nucleotides to be modified. Accordingly, in certain embodiments of any of the therapeutic oligonucleotides provided herein, all or substantially all of the nucleotides of an oligonucleotide are modified. In certain embodiments, more than half of the nucleotides are modified. In certain embodiments, less than half of the nucleotides are modified. Typically, with naked delivery, every sugar is modified at the 2′-position. These modifications may be reversible or irreversible. In some embodiments, an oligonucleotide as disclosed herein has a number and type of modified nucleotides sufficient to cause the desired characteristic (e.g., protection from enzymatic degradation, capacity to target a desired cell after in vivo administration, and/or thermodynamic stability).

### IV. Sugar Modifications

In some embodiments, a modified sugar (also referred to herein as a sugar analog) includes a modified deoxyribose or ribose moiety, e.g., in which one or more modifications occur at the 2′, 3′, 4′, and/or 5′ carbon position of the sugar. In some embodiments, a modified sugar may also include non-natural alternative carbon structures such as those present in locked nucleic acids (“LNA”) (see, e.g., Koshkin et al. (1998), Tetrahedron 54, 3607-3630), unlocked nucleic acids (“UNA”) (see, e.g., Snead et al. (2013), Molecular Therapy—Nucleic Acids, 2, e103), and bridged nucleic acids (“BNA”) (see, e.g., Imanishi and Obika (2002), The Royal Society of Chemistry, Chem. Commun., 1653-1659). Koshkin et al., Snead et al., and Imanishi and Obika are incorporated by reference herein for their disclosures relating to sugar modifications. In some embodiments, a nucleotide modification in a sugar comprises a 2′-modification. A 2′-modification may be 2′-aminoethyl, 2′-fluoro, 2′-O-methyl, 2′-O-methoxyethyl, and 2′-deoxy-2′-fluoro-β-d-arabinonucleic acid. Typically, the modification is 2′-fluoro, 2′-O-methyl, or 2′-O-methoxyethyl. In some embodiments, a modification in a sugar comprises a modification of the sugar ring, which may comprise modification of one or more carbons of the sugar ring. For example, a modification of a sugar of a nucleotide may comprise a 2′-oxygen of a sugar is linked to a 1′-carbon or 4′-carbon of the sugar, or a 2′-oxygen is linked to the 1′-carbon or 4′-carbon via an ethylene or methylene bridge. In some embodiments, a modified nucleotide has an acyclic sugar that lacks a 2′-carbon to 3′-carbon bond. In some embodiments, a modified nucleotide has a thiol group, e.g., in the 4′ position of the sugar.

In some embodiments, the terminal 3′-end group (e.g., a 3′-hydroxyl) is a phosphate group or other group, which can be used, for example, to attach linkers, adapters or labels or for the direct ligation of an oligonucleotide to another nucleic acid.

### V. 5′ Terminal Phosphates

In some embodiments, 5′-terminal phosphate groups of oligonucleotides enhance the interaction with Argonaut 2. However, oligonucleotides comprising a 5′-phosphate group may be susceptible to degradation via phosphatases or other enzymes, which can limit their bioavailability in vivo. In some embodiments, oligonucleotides include analogs of 5′ phosphates that are resistant to such degradation. In some embodiments, a phosphate analog may be oxymethylphosphonate, vinylphosphonate, or malonylphosphonate. In certain embodiments, the 5′ end of an oligonucleotide strand is attached to a chemical moiety that mimics the electrostatic and steric properties of a natural 5′-phosphate group (“phosphate mimic”) (see, e.g., Prakash et al. (2015), Nucleic Acids Res., Nucleic Acids Res. 2015 Mar. 31; 43(6): 2993-3011, the contents of which relating to phosphate analogs are incorporated herein by reference). Many phosphate mimics have been developed that can be attached to the 5′end (see, e.g., U.S. Pat. No. 8,927,513, the contents of which relating to phosphate analogs are incorporated herein by reference). Other modifications have been developed for the 5′ end of oligonucleotides (see, e.g., WO 2011/133871, the contents of which relating to phosphate analogs are incorporated herein by reference). In certain embodiments, a hydroxyl group is attached to the 5′ end of the oligonucleotide.

In some embodiments, an oligonucleotide has a phosphate analog at a 4′-carbon position of the sugar (referred to as a “4′-phosphate analog”). See, for example, U.S. Provisional Application Nos. 62/383,207, entitled 4′-Phosphate Analogs and Oligonucleotides Comprising the Same, filed on Sep. 2, 2016, and 62/393,401, filed on Sep. 12, 2016, entitled 4′-Phosphate Analogs and Oligonucleotides Comprising the Same, the contents of each of which relating to phosphate analogs are incorporated herein by reference. In some embodiments, an oligonucleotide provided herein comprises a 4′-phosphate analog at a 5′-terminal nucleotide. In some embodiments, a phosphate analog is an oxymethylphosphonate, in which the oxygen atom of the oxymethyl group is bound to the sugar moiety (e.g., at its 4′-carbon) or analog thereof. In other embodiments, a 4′-phosphate analog is a thiomethylphosphonate or an aminomethylphosphonate, in which the sulfur atom of the thiomethyl group or the nitrogen atom of the aminomethyl group is bound to the 4′-carbon of the sugar moiety or analog thereof. In certain embodiments, a 4′-phosphate analog is an oxymethylphosphonate. In some embodiments, an oxymethylphosphonate is represented by the formula —O—CH₂—PO(OH)₂ or —O—CH₂—PO(OR)₂, in which R is independently selected from H, CH₃, an alkyl group, CH₂CH₂CN, CH₂OCOC(CH₃)₃, CH₂OCH₂CH₂Si(CH₃)₃, or a protecting group. In certain embodiments, the alkyl group is CH₂CH₃. More typically, R is independently selected from H, CH₃, or CH₂CH₃.

In certain embodiments, a phosphate analog attached to the oligonucleotide is a methoxy phosphonate (MOP). In certain embodiments, a phosphate analog attached to the oligonucleotide is a 5′ mono-methyl protected MOP. In some embodiments, the following uridine nucleotide comprising a phosphate analog may be used, e.g., at the first position of a guide (antisense) strand:

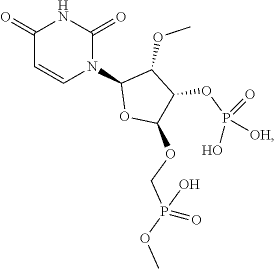 [SMILES: CO[C@@H]1[C@H](OP(=O)(O)O)[C@@H](OCP(=O)(O)OC)O[C@H]1n1ccc(=O)[nH]c1=O]

which modified nucleotide is referred to as [MePhosphonate-4O-mU] or 5′-Methoxy, Phosphonate-4′oxy-2′-O-methyluridine.

### VI. Modified Internucleoside Linkages

In some embodiments, phosphate modifications or substitutions may result in an oligonucleotide that comprises at least one (e.g., at least 1, at least 2, at least 3 or at least 5) modified internucleotide linkage. In some embodiments, any one of the oligonucleotides disclosed herein comprises 1 to 10 (e.g., 1 to 10, 2 to 8, 4 to 6, 3 to 10, 5 to 10, 1 to 5, 1 to 3 or 1 to 2) modified internucleotide linkages. In some embodiments, any one of the oligonucleotides disclosed herein comprises 1, 2, 3, 4, 5, 6, 7, 8, 9, or 10 modified internucleotide linkages.

A modified internucleotide linkage may be a phosphorothioate linkage, a phosphorothioate linkage, a phosphotriester linkage, a thionoalkylphosphonate linkage, a thionoalkylphosphotriester linkage, a phosphoramidite linkage, a phosphonate linkage or a boranophosphate linkage. In some embodiments, at least one modified internucleotide linkage of any one of the oligonucleotides as disclosed herein is a phosphorothioate linkage.

### VII. Base Modifications

In some embodiments, oligonucleotides provided herein have one or more modified nucleobases. In some embodiments, modified nucleobases (also referred to herein as base analogs) are linked at the 1′ position of a nucleotide sugar moiety. In certain embodiments, a modified nucleobase is a nitrogenous base. In certain embodiments, a modified nucleobase does not contain a nitrogen atom. See e.g., U.S. Published Patent Application No. 20080274462. In some embodiments, a modified nucleotide comprises a universal base. However, in certain embodiments, a modified nucleotide does not contain a nucleobase (abasic).

In some embodiments, a universal base is a heterocyclic moiety located at the 1′ position of a nucleotide sugar moiety in a modified nucleotide, or the equivalent position in a nucleotide sugar moiety substitution that, when present in a duplex, can be positioned opposite more than one type of base without substantially altering the structure of the duplex. In some embodiments, compared to a reference single-stranded nucleic acid (e.g., oligonucleotide) that is fully complementary to a target nucleic acid, a single-stranded nucleic acid containing a universal base forms a duplex with the target nucleic acid that has a lower Tₘ than a duplex formed with the complementary nucleic acid. However, in some embodiments, compared to a reference single-stranded nucleic acid in which the universal base has been replaced with a base to generate a single mismatch, the single-stranded nucleic acid containing the universal base forms a duplex with the target nucleic acid that has a higher Tₘ than a duplex formed with the nucleic acid comprising the mismatched base.

Non-limiting examples of universal-binding nucleotides include inosine, 1-β-D-ribofuranosyl-5-nitroindole, and/or 1-β-D-ribofuranosyl-3-nitropyrrole (US Pat. Appl. Publ. No. 20070254362 to Quay et al.; Van Aerschot et al., An acyclic 5-nitroindazole nucleoside analogue as ambiguous nucleoside, Nucleic Acids Res. 1995 Nov. 11; 23(21):4363-70; Loakes et al., 3-Nitropyrrole and 5-nitroindole as universal bases in primers for DNA sequencing and PCR, Nucleic Acids Res. 1995 Jul. 11; 23(13):2361-6; Loakes and Brown, 5-Nitroindole as an universal base analogue, Nucleic Acids Res. 1994 Oct. 11; 22(20):4039-43. Each of the foregoing is incorporated by reference herein for their disclosures relating to base modifications).

### VIII. Reversible Modifications

While certain modifications to protect an oligonucleotide from the in vivo environment before reaching target cells can be made, they can reduce the potency or activity of the oligonucleotide once it reaches the cytosol of the target cell. Reversible modifications can be made such that the molecule retains desirable properties outside of the cell, which are then removed upon entering the cytosolic environment of the cell. Reversible modification can be removed, for example, by the action of an intracellular enzyme or by the chemical conditions inside of a cell (e.g., through reduction by intracellular glutathione).

In some embodiments, a reversibly modified nucleotide comprises a glutathione-sensitive moiety. Typically, nucleic acid molecules have been chemically modified with cyclic disulfide moieties to mask the negative charge created by the internucleotide diphosphate linkages and improve cellular uptake and nuclease resistance. See U.S. Published Application No. 2011/0294869 originally assigned to Traversa Therapeutics, Inc. (“Traversa”), PCT Publication No. WO 2015/188197 to Solstice Biologics, Ltd. (“Solstice”), Meade et al., Nature Biotechnology, 2014, 32:1256-1263 (“Meade”), PCT Publication No. WO 2014/088920 to Merck Sharp & Dohme Corp, each of which are incorporated by reference for their disclosures of such modifications. This reversible modification of the internucleotide diphosphate linkages is designed to be cleaved intracellularly by the reducing environment of the cytosol (e.g. glutathione). Earlier examples include neutralizing phosphotriester modifications that were reported to be cleavable inside cells (Dellinger et al. J. Am. Chem. Soc. 2003, 125:940-950). In some embodiments, such a reversible modification allows protection during in vivo administration (e.g., transit through the blood and/or lysosomal/endosomal compartments of a cell) where the oligonucleotide will be exposed to nucleases and other harsh environmental conditions (e.g., pH). When released into the cytosol of a cell where the levels of glutathione are higher compared to extracellular space, the modification is reversed and the result is a cleaved oligonucleotide. Using reversible, glutathione sensitive moieties, it is possible to introduce sterically larger chemical groups into the oligonucleotide of interest as compared to the options available using irreversible chemical modifications. This is because these larger chemical groups will be removed in the cytosol and, therefore, should not interfere with the biological activity of the oligonucleotides inside the cytosol of a cell. As a result, these larger chemical groups can be engineered to confer various advantages to the nucleotide or oligonucleotide, such as nuclease resistance, lipophilicity, charge, thermal stability, specificity, and reduced immunogenicity. In some embodiments, the structure of the glutathione-sensitive moiety can be engineered to modify the kinetics of its release.

In some embodiments, a glutathione-sensitive moiety is attached to the sugar of the nucleotide. In some embodiments, a glutathione-sensitive moiety is attached to the 2′-carbon of the sugar of a modified nucleotide. In some embodiments, the glutathione-sensitive moiety is located at the 5′-carbon of a sugar, particularly when the modified nucleotide is the 5′-terminal nucleotide of the oligonucleotide. In some embodiments, the glutathione-sensitive moiety is located at the 3′-carbon of a sugar, particularly when the modified nucleotide is the 3′-terminal nucleotide of the oligonucleotide. In some embodiments, the glutathione-sensitive moiety comprises a sulfonyl group. See, e.g., U.S. Prov. Appl. No. 62/378,635, entitled Compositions Comprising Reversibly Modified Oligonucleotides and Uses Thereof, which was filed on Aug. 23, 2016, the contents of which are incorporated by reference herein for its relevant disclosures.

### IX. Targeting Ligands

In some embodiments, it may be desirable to target the oligonucleotides of the disclosure to one or more cells or one or more organs. Such a strategy may help to avoid undesirable effects in other organs, or may avoid undue loss of the oligonucleotide to cells, tissue or organs that would not benefit for the oligonucleotide. Accordingly, in some embodiments, oligonucleotides disclosed herein may be modified to facilitate targeting of a particular tissue, cell or organ, e.g., to facilitate delivery of the oligonucleotide to the liver. In certain embodiments, oligonucleotides disclosed herein may be modified to facilitate delivery of the oligonucleotide to the hepatocytes of the liver. In some embodiments, an oligonucleotide comprises a nucleotide that is conjugated to one or more targeting ligands.

A targeting ligand may comprise a carbohydrate, amino sugar, cholesterol, peptide, polypeptide, protein or part of a protein (e.g., an antibody or antibody fragment) or lipid. In some embodiments, a targeting ligand is an aptamer. For example, a targeting ligand may be an RGD peptide that is used to target tumor vasculature or glioma cells, CREKA peptide to target tumor vasculature or stoma, transferrin, lactoferrin, or an aptamer to target transferrin receptors expressed on CNS vasculature, or an anti-EGFR antibody to target EGFR on glioma cells. In certain embodiments, the targeting ligand is one or more GalNAc moieties.

In some embodiments, 1 or more (e.g., 1, 2, 3, 4, 5 or 6) nucleotides of an oligonucleotide are each conjugated to a separate targeting ligand. In some embodiments, 2 to 4 nucleotides of an oligonucleotide are each conjugated to a separate targeting ligand. In some embodiments, targeting ligands are conjugated to 2 to 4 nucleotides at either ends of the sense or antisense strand (e.g., ligands are conjugated to a 2 to 4 nucleotide overhang or extension on the 5′ or 3′ end of the sense or antisense strand) such that the targeting ligands resemble bristles of a toothbrush and the oligonucleotide resembles a toothbrush. For example, an oligonucleotide may comprise a stem-loop at either the 5′ or 3′ end of the sense strand and 1, 2, 3 or 4 nucleotides of the loop of the stem may be individually conjugated to a targeting ligand.

In some embodiments, it is desirable to target an oligonucleotide that reduces the expression of HBV antigen to the hepatocytes of the liver of a subject. Any suitable hepatocyte targeting moiety may be used for this purpose.

GalNAc is a high affinity ligand for asialoglycoprotein receptor (ASGPR), which is primarily expressed on the sinusoidal surface of hepatocyte cells and has a major role in binding, internalization, and subsequent clearance of circulating glycoproteins that contain terminal galactose or N-acetylgalactosamine residues (asialoglycoproteins). Conjugation (either indirect or direct) of GalNAc moieties to oligonucleotides of the instant disclosure may be used to target these oligonucleotides to the ASGPR expressed on these hepatocyte cells.

In some embodiments, an oligonucleotide of the instant disclosure is conjugated directly or indirectly to a monovalent GalNAc. In some embodiments, the oligonucleotide is conjugated directly or indirectly to more than one monovalent GalNAc (i.e., is conjugated to 2, 3, or 4 monovalent GalNAc moieties, and is typically conjugated to 3 or 4 monovalent GalNAc moieties). In some embodiments, an oligonucleotide of the instant disclosure is conjugated to one or more bivalent GalNAc, trivalent GalNAc, or tetravalent GalNAc moieties.

In some embodiments, 1 or more (e.g., 1, 2, 3, 4, 5 or 6) nucleotides of an oligonucleotide are each conjugated to a GalNAc moiety. In some embodiments, 2 to 4 nucleotides of the loop (L) of the stem-loop are each conjugated to a separate GalNAc. In some embodiments, targeting ligands are conjugated to 2 to 4 nucleotides at either ends of the sense or antisense strand (e.g., ligands are conjugated to a 2 to 4 nucleotide overhang or extension on the 5′ or 3′ end of the sense or antisense strand) such that the GalNAc moieties resemble bristles of a toothbrush and the oligonucleotide resembles a toothbrush. For example, an oligonucleotide may comprise a stem-loop at either the 5′ or 3′ end of the sense strand and 1, 2, 3 or 4 nucleotides of the loop of the stem may be individually conjugated to a GalNAc moiety. In some embodiments, GalNAc moieties are conjugated to a nucleotide of the sense strand. For example, four GalNAc moieties can be conjugated to nucleotides in the tetraloop of the sense strand, where each GalNAc moiety is conjugated to one nucleotide.

In some embodiments, an oligonucleotide herein comprises a monovalent GalNAc attached to a Guanidine nucleotide, referred to as [ademG-GalNAc] or 2′-aminodiethoxymethanol-Guanidine-GalNAc, as depicted below:

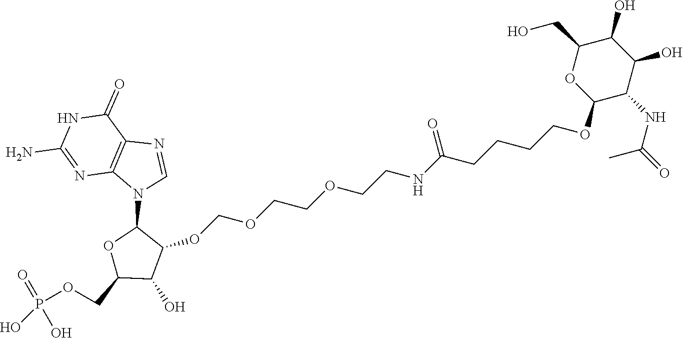 [SMILES: CC(=O)N[C@@H]1[C@@H](OCCCCC(=O)NCCOCCOCO[C@@H]2[C@H](O)[C@@H](COP(=O)(O)O)O[C@H]2n2cnc3c(=O)[nH]c(N)nc32)O[C@@H](CO)[C@@H](O)[C@H]1O]

In some embodiments, an oligonucleotide herein comprises a monovalent GalNAc attached to an adenine nucleotide, referred to as [ademA-GalNAc] or 2′-aminodiethoxymethanol-Adenine-GalNAc, as depicted below.

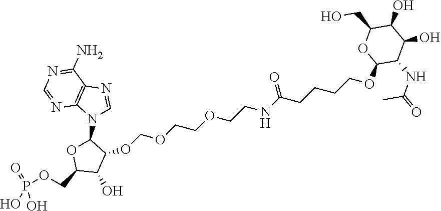

An example of such conjugation is shown below for a loop comprising from 5′ to 3′ the nucleotide sequence GAAA (L=linker, X=heteroatom) stem attachment points are shown. In the chemical formula,

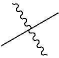

is an attachment point to the oligonucleotide strand.

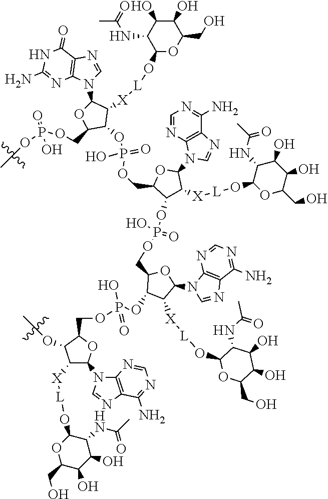

Appropriate methods or chemistry (e.g., click chemistry) can be used to link a targeting ligand to a nucleotide. In some embodiments, a targeting ligand is conjugated to a nucleotide using a click linker. In some embodiments, an acetal-based linker is used to conjugate a targeting ligand to a nucleotide of any one of the oligonucleotides described herein. Acetal-based linkers are disclosed, for example, in International Patent Application Publication Number WO2016100401 A1, which published on Jun. 23, 2016, and the contents of which relating to such linkers are incorporated herein by reference. In some embodiments, the linker is a labile linker. However, in other embodiments, the linker is fairly stable.

An example is shown below for a loop comprising from 5′ to 3′ the nucleotides GAAA, in which GalNAc moieties are attached to nucleotides of the loop using an acetal linker. In the chemical formula,

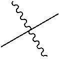

is an attachment point to the oligonucleotide strand.

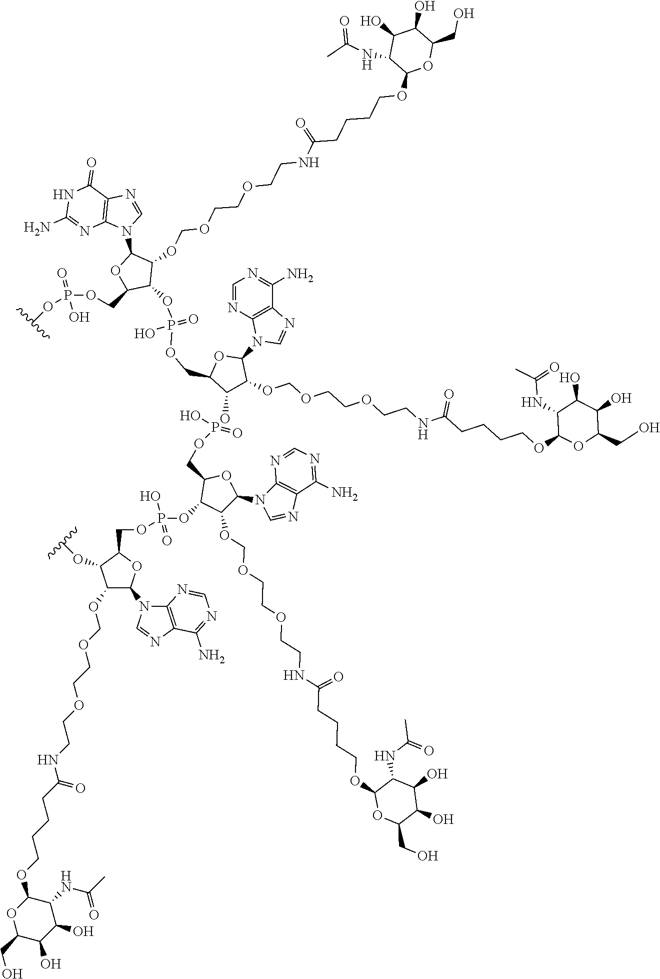 [SMILES: *O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(=O)[nH]c(N)nc32)O[C@@H]1COP(=O)(O)O*]

### Anti-PDL1 Antisense Oligonucleotide

In an embodiment, a therapeutic used in the pharmaceutical combination of the present invention is an anti-PDL1 antisense oligonucleotide.

In an embodiment, the anti-PDL1 antisense oligonucleotide is an N-acetylgalactosamine (GalNAc)-conjugated locked nucleic acid (LNA) single-stranded oligonucleotide (SSO) that induces RNAseH-mediated degradation of PDL1 mRNA.

In an embodiment, the anti-PDL1 antisense oligonucleotides in the pharmaceutical combination of the present invention is disclosed in WO2017/157899 which is fully incorporated herein by reference.

In a preferred embodiment, the anti-PDL1 antisense oligonucleotide in the pharmaceutical combination of the present invention is CMP ID NO: 768_2 disclosed in WO2017/157899 or a pharmaceutically acceptable salt thereof.

In an embodiment, the anti-PDL1 antisense oligonucleotide in the pharmaceutical combination of the present invention comprises the sequence CCTATTTAACATCAGAC (SEQ ID NO: 11). In a preferred embodiment, the anti-PDL1 antisense oligonucleotide in the pharmaceutical combination of the present invention has the formula GN2-C6ₒcₒaₒCCtatttaacatcAGAC, wherein C6 represents an amino alkyl group with 6 carbons, capital letters represent beta-D-oxy LNA nucleosides, lowercase letters represent DNA nucleosides, all LNA C are 5-methyl cytosine, subscript ₒ represents a phosphodiester nucleoside linkage, and unless otherwise indicated, all internucleoside linkages are phosphorothioate internucleoside linkages, and wherein GN2 represents the following trivalent GalNAc cluster:

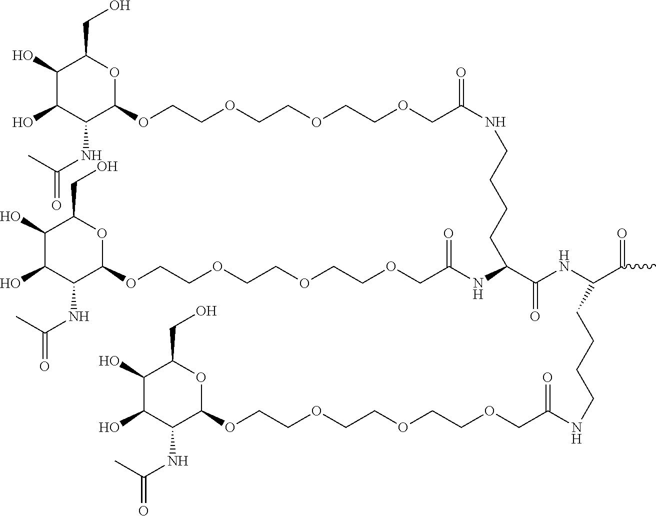

and further wherein the wavy line of the trivalent GalNAc cluster illustrates the site of conjugation of the trivalent GalNAc cluster to the C6 amino alkyl group; or a pharmaceutically acceptable salt thereof. This definition of the anti-PDL1 antisense oligonucleotide used in the pharmaceutical combinations of the present invention is referred to herein as “T2” or “Therapeutic T2”.

In an embodiment, the anti-PDL1 antisense oligonucleotide is administered subcutaneously. In an embodiment, the anti-PDL1 antisense oligonucleotide is administered in a dose or doses of about 0.1 mg/kg to about 35 mg/kg, or about 0.1 mg/kg to about 15 mg/kg, or about 0.1 mg/kg to about 10 mg/kg, or about 0.2 m/kg to about 10 mg/kg, or about 0.25 mg/kg to about 10 mg/kg, or about 0.1 mg/kg to about 5 mg/kg, or about 0.2 mg/kg to about 5 mg/kg, or about 0.25 mg/kg to about 5 mg/kg.

In an embodiment, the anti-PDL1 antisense oligonucleotide is administered in a dose or doses of about 7 mg/kg to about 35 mg/kg.

In an embodiment, the doses of the anti-PDL1 antisense oligonucleotide are administered once a week, once every two weeks, once every three weeks or once a month.

In a further preferred embodiment of the pharmaceutical combination of the present invention, particularly when further comprising an RNAi oligonucleotide targeting HBV, the anti-PDL1 antisense oligonucleotide is administered in up to five doses. Preferably, each dose is about 3 mg/kg. Preferably, the doses are administered Q2W (every two weeks).

### I. Antisense Oligonucleotide Modifications

The modifications discussed in this section are especially preferable for implementation in the antisense oligonucleotide of the present invention.

It is understood that the contiguous nucleobase sequences (motif sequence) can be modified to for example increase nuclease resistance and/or binding affinity to the target nucleic acid.

In one embodiment the contiguous nucleobase sequence of the oligonucleotide comprises at least one modified internucleoside linkage. Suitable internucleoside modifications are described in the “Definitions” section under “Modified internucleoside linkage”. It is advantageous if at least 75%, such as all, the internucleoside linkages within the contiguous nucleotide sequence are internucleoside linkages. In some embodiments all the internucleotide linkages in the contiguous sequence of the oligonucleotide are phosphorothioate linkages.

The oligonucleotides of the invention are designed with modified nucleosides and DNA nucleosides. Advantageously, high affinity modified nucleosides are used.

In an embodiment, the oligonucleotide comprises at least 3 modified nucleosides, such as at least 4, at least 5, at least 6, at least 7, at least 8, at least 9, at least 10, at least 11, at least 12, at least 13, at least 14, at least 15 or at least 16 modified nucleosides. In an embodiment the oligonucleotide comprises from 3 to 8 modified nucleosides, such as from 4 to 6 modified nucleosides, such as 4, 5 or 6 nucleosides, such as from 5 or 6 modified nucleosides. Suitable modifications are described in the “Definitions” section under “modified nucleoside”, “high affinity modified nucleosides”, “sugar modifications”, “2′ sugar modifications” and Locked nucleic acids (LNA)”.

In an embodiment, the oligonucleotide comprises one or more sugar modified nucleosides, such as 2′ sugar modified nucleosides. Preferably the oligonucleotide of the invention comprises one or more 2′ sugar modified nucleoside independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA nucleosides. It is advantageous if one or more or all of the modified nucleoside(s) is a locked nucleic acid (LNA).

In some embodiments, the oligonucleotide of the invention, such as the contiguous nucleotide sequence, comprises at least one LNA nucleoside, such as 1, 2, 3, 4, 5, 6, 7, or 8 LNA nucleosides, such as from 2 to 6 LNA nucleosides, such as from 3 to 6 LNA nucleosides, 4 to 6 LNA nucleosides or 4, 5 or 6 LNA nucleosides.

In some embodiments, at least 75% of the modified nucleosides in the oligonucleotide are LNA nucleosides, such as at least 80%, such as at least 85%, such as at least 90% of the modified nucleosides are LNA nucleosides. In a still further embodiment all the modified nucleosides in the oligonucleotide are LNA nucleosides. In a further embodiment, the LNA nucleosides are selected from beta-D-oxy-LNA, thio-LNA, amino-LNA, oxy-LNA, ScET and/or ENA in either the beta-D or alpha-L configurations or combinations thereof. In a further embodiment, all LNA nucleosides are beta-D-oxy-LNA. In a further embodiment cytosine units are 5-methyl-cytosine.

It is advantageous for the nuclease stability of the oligonucleotide or contiguous nucleotide sequence to have at least 1 LNA nucleoside at the 5′ end and at least 2 LNA nucleosides at the 3′ end of the nucleotide sequence.

### TLR7 Agonist

In an embodiment, a therapeutic used in the pharmaceutical combination of the present invention is a TLR7 agonist.

In an embodiment, the TLR7 agonist in the pharmaceutical combination of the present invention is a 3-substituted 5-amino-6H-thiazolo[4,5-d]pyrimidine-2, 7-dione compound, that has Toll-like receptor agonism activity, or a prodrug thereof. WO 2006/066080, WO 2016/055553 and WO 2016/091698 describe such TLR7 agonists and their prodrug and their manufacture (hereby incorporated by reference).

In an embodiment the TLR7 agonist in the pharmaceutical combination of the present invention is represented by formula (I):

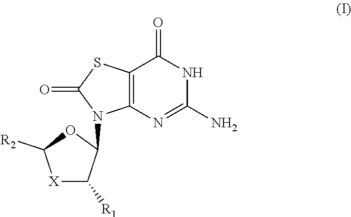

wherein X is CH₂ or S;
R₁ is —OH or —H and
R₂ is 1-hydroxypropyl or hydroxymethyl;
   or formula (II):

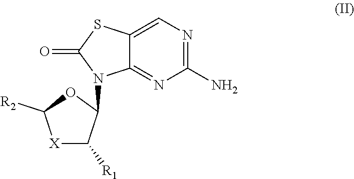

wherein X is CH₂ or S;
R₁ is —OH or —H or acetoxy and
R₂ is 1-acetoxypropyl or 1-hydroxypropyl or 1-hydroxymethyl or acetoxy(cyclopropyl)methyl or acetoxy(propyn-1-yl)methyl,
   or a pharmaceutically acceptable salt, enantiomer or diastereomer thereof. Compounds of formula (I) are active TLR7 agonists.

In an embodiment, a subset of the active TLR7 agonist of formula (I) in the pharmaceutical combination of the present invention are represented by formula (V):

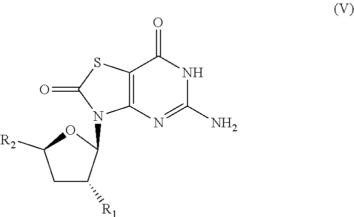

wherein R₁ is —OH and R₂ is 1-hydroxypropyl or hydroxymethyl,
   or a pharmaceutically acceptable salt, enantiomer or diastereomer thereof.

In an embodiment, the substituent at R₂ in formula (I) or (V) is selected from:

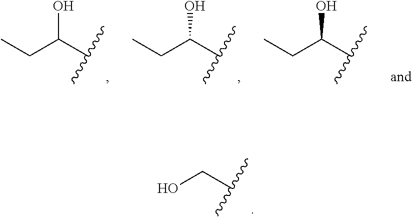

Compounds of formula (II) are TLR7 agonist prodrugs. In one embodiment the prodrug is a single prodrug with substituent at R₂ selected from:

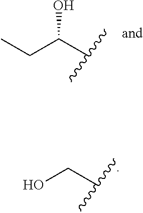

In an embodiment, the prodrug is a double prodrug with substituent at R₂ selected from:

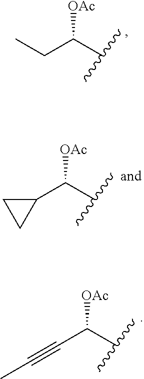

In an embodiment, a subset of the TLR7 agonist prodrugs of formula (II) in the pharmaceutical combination of the present invention is represented by formula (III):

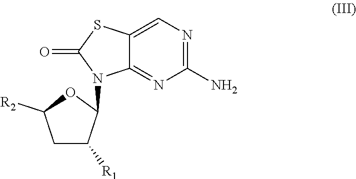

wherein R₁ is —OH or acetoxy and R₂ is 1-acetoxypropyl or 1-hydroxypropyl or 1-hydroxymethyl or

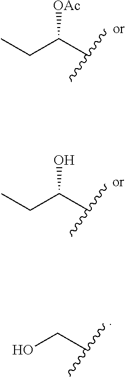

or a pharmaceutically acceptable salt, enantiomer or diastereomer thereof;
or formula (IV):

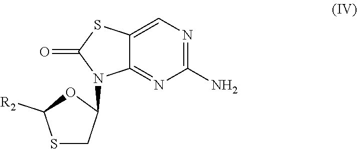

wherein R₁ is acetoxy(cyclopropyl)methyl or acetoxy(propyn-1-yl)methyl or

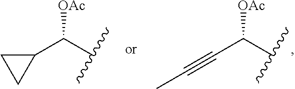

or a pharmaceutically acceptable salt, enantiomer or diastereomer thereof.

The compounds of formula (IV) are double prodrugs as is the compound of formula (III) where R₁ is OH and R₂ is 1-acetoxypropyl. The compound of formula (III) where R₁ is acetoxy and R₂ is a triple prodrug.

After administration, compounds of formula (II), (III) or formula (IV) are metabolized into their active forms which are useful TLR7 agonists.

In an embodiment, the TLR7 agonist in the pharmaceutical combination of the present invention is selected from the group consisting of:
[(1S)-1-[(2S,4R,5R)-5-(5-amino-2-oxo-thiazolo[4,5-d]pyrimidin-3-yl)-4-hydroxy-tetrahydrofuran-2-yl]propyl]acetate (CMP ID NO: VI);
5-amino-3-[(2R,3R,5S)-3-hydroxy-5-[(1S)-1-hydroxypropyl]tetrahydrofuran-2-yl]-6H-thiazolo[4,5-d]pyrimidine-2,7-dione (CMP ID NO: VII);
5-amino-3-[(2R,3R,5S)-3-hydroxy-5-[(1S)-1-hydroxypropyl]tetrahydrofuran-2-yl]thiazolo[4,5-d]pyrimidin-2-one (CMP ID NO: VIII);
5-amino-3-(3′-deoxy-β-D-ribofuranosyl)-3H-thiazolo[4,5-d]pyrimidin-2-one (CMP ID NO: IX);
5-amino-3-(2′-O-acetyl-3′-deoxy-β-D-ribofuranosyl)-3H-thiazolo[4,5-d]pyrimidin-2-one (CMP ID NO: X);
5-amino-3-(3′-deoxy-3-D-ribofuranosyl)-3H,6H-thiazolo[4,5-d]pyrimidin-2,7-dione (CMP ID NO: XI);
[(S)-[(2S,5R)-5-(5-amino-2-oxo-thiazolo[4,5-d]pyrimidin-3-yl)-1,3-oxathiolan-2-yl]-cyclopropyl-methyl]acetate (CMP ID NO: XII); and
(1S)-1-[(2S,5R)-5-(5-amino-2-oxo-thiazolo[4,5-d]pyrimidin-3-yl)-1,3-oxathiolan-2-yl]but-2-ynyl]acetate (CMP ID NO: XIII)
   and their pharmaceutically acceptable salt, enantiomer or diastereomer.

Table 1 lists the TLR7 agonists in embodiments of the pharmaceutical combination of the present invention, including reference to documents that describe their manufacture.

TABLE 1
TLR7 agonist compounds identified with individual compound identification numbers
(CMP ID NO).

| CMP | | | |
|---|---|---|---|
| ID | | | |
| NO | Compound Name | Structure | reference |
| VI | [(1S)-1-[(2S,4R,5R)-5-(5- amino-2-oxo-thiazolo[4,5- d]pyrimidin-3-yl)-4-hydroxy- tetrahydrofuran-2-yl]propyl] acetate | 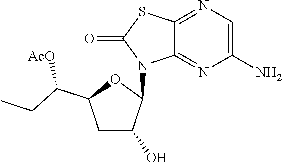 [SMILES: CC[C@H](OC(C)=O)[C@@H]1C[C@@H](O)[C@H](n2c(=O)sc3ncc(N)nc32)O1] | WO2016091698 |
| VII | 5-amino-3-[(2R,3R,5S)-3- hydroxy-5-[(1S)-1- hydroxypropyl]tetrahydrofuran- 2-yl]-6H-thiazolo [4,5-d]pyrimidine-2,7-dione | 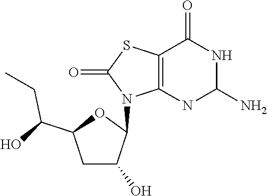 [SMILES: CC[C@H](O)[C@@H]1C[C@@H](O)[C@H](n2c3c(sc2=O)C(=O)NC(N)N3)O1] | WO2016091698 |
| VIII | 5-amino-3-[(2R,3R,5S)-3- hydroxy-5-[(1S)-1- hydroxypropyl]tetrahydrofuran- 2-yl]thiazolo[4,5-d]pyrimidin- 2-one | 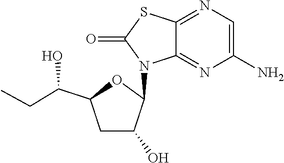 [SMILES: CC[C@H](O)[C@@H]1C[C@@H](O)[C@H](n2c(=O)sc3ncc(N)nc32)O1] | WO2016091698 |
| IX | 5-amino-3-(3′-deoxy-β-D- ribofuranosyl)-3H- thiazolo[4,5-d]pyrimidin-2-one | 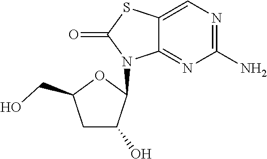 [SMILES: Nc1ncc2sc(=O)n([C@@H]3O[C@H](CO)C[C@H]3O)c2n1] | WO2006066080 |
| X | 5-amino-3-(2′-O-acetyl-3′- deoxy-β-D-ribofuranosyl)-3H- thiazolo[4,5-d]pyrimidin-2-one | 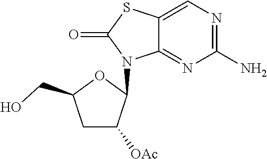 [SMILES: CC(=O)O[C@@H]1C[C@@H](CO)O[C@H]1n1c(=O)sc2cnc(N)nc21] | WO2006066080 |
| XI | 5-amino-3-(3′-deoxy-β-D- ribofuranosyl)-3H,6H- thiazolo[4,5-d]pyrimidin-2,7- dione | 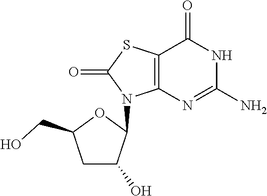 [SMILES: Nc1nc2c(sc(=O)n2[C@@H]2O[C@H](CO)C[C@H]2O)c(=O)[nH]1] | WO2006066080 |
| XII | [(S)-[(2S,5R)-5-(5-amino-2- oxo-thiazolo[4,5-d]pyrimidin- 3-yl)-1,3-oxathiolan-2-yl]- cyclopropyl-methyl]acetate | 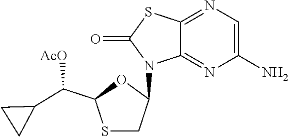 [SMILES: CC(=O)O[C@@H](C1CC1)[C@H]1O[C@@H](n2c(=O)sc3ncc(N)nc32)CS1] | WO2016055553 |
| XIII | (1S)-1-[(2S,5R)-5-(5-amino-2- oxo-thiazolo[4,5-d]pyrimidin- 3-yl)-1,3-oxathiolan-2-yl]but- 2-ynyl]acetate | 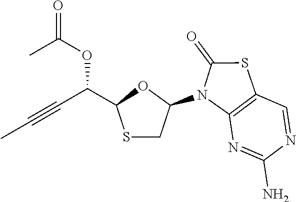 [SMILES: CC#C[C@H](OC(C)=O)[C@H]1O[C@@H](n2c(=O)sc3cnc(N)nc32)CS1] | WO2016055553 |

In a particularly preferred embodiment in the pharmaceutical combination of the present invention, the TLR7 agonist is CMP ID NO: VI. This definition of TLR7 agonist used in the pharmaceutical combinations of the present invention is referred to herein as “T3” or

### “Therapeutic T3”.

In an embodiment, the TLR7 agonist is administered orally.

In one embodiment, T3 is administered orally as a unit dose ranging between 150 and 170 mg every other day (QOD) for 8 to 26 weeks such as 10 to 24 weeks such as 12 or 13 weeks followed by a weekly administration (QW) for 24 to 48 weeks such as 30 to 40 weeks such as 35 weeks. The number of doses administered of T3 is between 60 and 100 doses, such as between 75 and 90 doses, such as 81, 82, 83 or 84 doses throughout the treatment period. Preferably, the TLR7 agonist is administered over a period of not more than twelve weeks. In one embodiment, a pharmaceutical combination of the present invention which comprises T1 or T2 as well as T3, T1 and T3, or T2 and T3, are administered less than a month apart, such as less than a week apart, such as two days apart, such as on the same day.

In an embodiment, the TLR7 agonist in the pharmaceutical combination of the invention is administered enterally (such as orally or through the gastrointestinal tract). The TLR7 agonist compounds in the present invention may be administered in unit doses of any convenient administrative form, e.g., tablets, powders, capsules, solutions, dispersions, suspensions, syrups, sprays, suppositories, gels, emulsions. In particular oral unit dosage forms, such as tablets and capsules, can be used. In one example, the pharmaceutically effective amount of the TLR7 agonist compound of the invention will be in the range of about 75-250 mg, such as 100 to 200 mg such as 150 to 170 mg pr. dose. The administration can be daily, every other day (QOD) or weekly (QW).

In a preferred embodiment of pharmaceutical combinations of the invention which comprise a TLR7 agonist, the TLR7 agonist is administered in a dose of at least about 100 mg, or about 100 mg, or preferably about 150 mg. In an embodiment, the TLR7 agonist is administered at least QW (weekly), or QW, or more preferably QOD (every other day).

Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005.

### Interferon-Alpha

In an embodiment, a therapeutic used in the pharmaceutical combination of the present invention is interferon-alpha (IFNα).

In various embodiments, the interferon-alpha in the pharmaceutical combination of the present invention may be interferon alpha-2b, interferon alpha-2a, and interferon alphacon-1 (pegylated and unpegylated).

In further various embodiments, IFN-α in the pharmaceutical combination of the present invention is Pegasys® (Roche), PEG-Intron® (Merck& Co., Inc.) or Y-pegylated recombinant interferon alpha-2a (YPEG-IFNα-2a, Xiamen Amoytop Biotech Co., Ltd).

In an embodiment, the IFNα in the pharmaceutical combination of the present invention is pegylated IFNα. This definition of IFNα used in the pharmaceutical combinations of the present invention is referred to herein as “T4” or “Therapeutic T4”.

In an embodiment, the interferon-alpha is administered subcutaneously.

### Anti-HBV Antibody

In an embodiment, a therapeutic used in the pharmaceutical combination of the present invention is an anti-HBV antibody.

In an embodiment, the anti-HBV antibody in the pharmaceutical combination of the present invention is an antibody that binds to hepatitis B surface antigen (anti-HBsAg).

In an embodiment, the combination comprising an oligonucleotide therapeutic and an anti-HBV antibody may lead to seroclearance of HBsAg in the patient.

In an embodiment, the anti-HBV antibody in the pharmaceutical combination of the present invention is monoclonal.

In an embodiment, the anti-HBV antibody in the pharmaceutical combination of the present invention is monoclonal is human.

In an embodiment, the anti-HBV antibody in the pharmaceutical combination of the present invention is an anti-HBsAg monoclonal antibody. This definition of anti-HBV antibody used in the pharmaceutical combinations of the present invention is referred to herein as “T5” or “Therapeutic T5”.

In a preferred embodiment of the pharmaceutical combination of the present invention that comprises T5, specifically in an embodiment of any of the combinations C4, C11, C17, C22, C27, C28, C29, C30, C39, C45, C50, C66, C71, C86, C55, C56, C57, C58, C76, C77, C78, C79, C91, C92, C93, C94, C101, C102, C103, C104, C111, C112, C113, C114, C115 or C116 identified in Tables 2 and 3, T5 is an anti-HBsAg antibody that comprises a heavy chain variable domain (VH) comprising (a) CDR-H1 comprising the amino acid sequence of NYGMQ (SEQ ID NO:12), (b) CDR-H2 comprising the amino acid sequence of IIWADGTKQYYGDSVKG (SEQ ID NO:13), and (c) CDR-H3 comprising the amino acid sequence of DGLYASAPNDV (SEQ ID NO:14), and a light chain variable domain (VL) comprising (d) CDR-L1 comprising the amino acid sequence of RASQRISTYLN (SEQ ID NO:15), (e) CDR-L2 comprising the amino acid sequence of GASSLQS (SEQ ID NO:16), and (f) CDR-L3 comprising the amino acid sequence of QQTYTLPPN (SEQ ID NO:17).

In an embodiment of the pharmaceutical combination of the present invention that comprises T5, specifically in an embodiment of any of the combinations C4, C11, C17, C22, C27, C28, C29, C30, C39, C45, C50, C66, C71, C86, C55, C56, C57, C58, C76, C77, C78, C79, C91, C92, C93, C94, C101, C102, C103, C104, C111, C112, C113, C114, C115 or C116 identified in Tables 2 and 3, T5 is an anti-HBsAg antibody that comprises a heavy chain variable domain (VH) comprising (a) CDR-H1 comprising the amino acid sequence of SYAMS (SEQ ID NO:18), (b) CDR-H2 comprising the amino acid sequence of AFSGTGGSTYYADSVKG (SEQ ID NO:19), and (c) CDR-H3 comprising the amino acid sequence of DPGHTSNWRDNYQYYQMDV (SEQ ID NO:20), and a light chain variable domain (VL) comprising (d) CDR-L1 comprising the amino acid sequence of RASQGIRNDLG (SEQ ID NO:21), (e) CDR-L2 comprising the amino acid sequence of AASSLQS (SEQ ID NO:22), and (f) CDR-L3 comprising the amino acid sequence of LQHNSYPRT (SEQ ID NO:23).

In an embodiment of the pharmaceutical combination of the present invention that comprises T5, specifically in an embodiment of any of the combinations C4, C11, C17, C22, C27, C28, C29, C30, C39, C45, C50, C66, C71, C86, C55, C56, C57, C58, C76, C77, C78, C79, C91, C92, C93, C94, C101, C102, C103, C104, C111, C112, C113, C114, C115 or C116 identified in Tables 2 and 3, T5 is an anti-HBsAg antibody that comprises a heavy chain variable domain (VH) comprising (a) CDR-H1 comprising the amino acid sequence of NYHIH (SEQ ID NO:24), (b) CDR-H2 comprising the amino acid sequence of IINPRRLSTAYAPKFQG (SEQ ID NO:25), and (c) CDR-H3 comprising the amino acid sequence of DAGDDTSGPFDS (SEQ ID NO:26), and a light chain variable domain (VL) comprising (d) CDR-L1 comprising the amino acid sequence of RASQSINTWLA (SEQ ID NO:27), (e) CDR-L2 comprising the amino acid sequence of KASSLES (SEQ ID NO:28), and (f) CDR-L3 comprising the amino acid sequence of QQYNTFS (SEQ ID NO:29).

In an embodiment of the pharmaceutical combination of the present invention that comprises T5, specifically in an embodiment of any of the combinations C4, C11, C17, C22, C27, C28, C29, C30, C39, C45, C50, C66, C71, C86, C55, C56, C57, C58, C76, C77, C78, C79, C91, C92, C93, C94, C101, C102, C103, C104, C111, C112, C113, C114, C115 or C116 identified in Tables 2 and 3, T5 is an anti-HBsAg antibody that comprises a heavy chain variable domain (VH) comprising (a) CDR-H1 comprising the amino acid sequence of TNNWWS (SEQ ID NO:30), (b) CDR-H2 comprising the amino acid sequence of EIHHIGSTNYNPSLKS (SEQ ID NO:31), and (c) CDR-H3 comprising the amino acid sequence of GRLGITRDRYYFDS (SEQ ID NO:32), and a light chain variable domain (VL) comprising (d) CDR-L1 comprising the amino acid sequence of QASQDISNYLN (SEQ ID NO:33), (e) CDR-L2 comprising the amino acid sequence of DTSSLER (SEQ ID NO:34), and (f) CDR-L3 comprising the amino acid sequence of QQYYNLPHT (SEQ ID NO:35).

In a preferred embodiment of the pharmaceutical combination of the present invention that comprises T5, specifically in an embodiment of any of the combinations C4, C11, C17, C22, C27, C28, C29, C30, C39, C45, C50, C66, C71, C86, C55, C56, C57, C58, C76, C77, C78, C79, C91, C92, C93, C94, C101, C102, C103, C104, C111, C112, C113, C114, C115 or C116 identified in Tables 2 and 3, T5 is an anti-HBsAg antibody that comprises a heavy chain variable domain (VH) comprising the amino acid sequence of QVQLVESGGGVVQPGRSLRLSCEASGFTFSNYGMQWVRQAPGKGLEWVAIIWADGTKQYYG DSVKGRFTISRDNFKNTLYLQMNSLRGEDTAMYFCARDGLYASAPNDVWGQGTLVTVSS (SEQ ID NO: 39), and a light chain variable domain (VL) comprising the amino acid sequence of DIQMTQSPSSLSAYVGDRVTITCRASQRISTYLNWYHQRPGKSPSLLIYGASSLQSGVPSRFSA SASGTDFTLTISSLRPEDLGTYYCQQTYTLPPNSGGGTKVEIK (SEQ ID NO: 37).

In an embodiment of the pharmaceutical combination of the present invention that comprises T5, specifically in an embodiment of any of the combinations C4, C11, C17, C22, C27, C28, C29, C30, C39, C45, C50, C66, C71, C86, C55, C56, C57, C58, C76, C77, C78, C79, C91, C92, C93, C94, C101, C102, C103, C104, C111, C112, C113, C114, C115 or C116 identified in Tables 2 and 3, T5 is an anti-HBsAg antibody that comprises a heavy chain comprising the amino acid sequence of QVQLVESGGGVVQPGRSLRLSCEASGFTFSNYGMQWVRQAPGKGLEWVAIIWADGTKQYYG DSVKGRFTISRDNFKNTLYLQMNSLRGEDTAMYFCARDGLYASAPNDVWGQGTLVTVSSAST KGPSVFPLAPSSKSTSGGTAALGCLVKDYFPEPVTVSWNSGALTSGVHTFPAVLQSSGLYSLS SVVTVPSSSLGTQTYICNVNHKPSNTKVDKKVEPKSCDKTHTCPPCPAPELLGGPSVFLFPPKP KDTLYITREPEVTCVVVDVSHEDPEVKFNWYVDGVEVHNAKTKPREEQYNSTYRVVSVLTVLH QDWLNGKEYKCKVSNKALPAPIEKTISKAKGQPREPQVYTLPPSRDELTKNQVSLTCLVKGFYP SDIAVEWESNGQPENNYKTTPPVLDSDGSFFLYSKLTVDKSRWQQGNVFSCSVMHEALHNHY TQKSLSLSPG (SEQ ID NO: 38), and a light chain comprising the amino acid sequence of DIQMTQSPSSLSAYVGDRVTITCRASQRISTYLNWYHQRPGKSPSLLIYGASSLQSGVPSRFSA SASGTDFTLTISSLRPEDLGTYYCQQTYTLPPNSGGGTKVEIKRTVAAPSVFIFPPSDEQLKSGT ASVVCLLNNFYPREAKVQWKVDNALQSGNSQESVTEQDSKDSTYSLSSTLTLSKADYEKHKVY ACEVTHQGLSSPVTKSFNRGEC (SEQ ID NO: 36).

In an embodiment, the anti-HBV antibody is administered subcutaneously.

Antibody which Antagonises PD1 Signalling

In an embodiment, a therapeutic used in the pharmaceutical combination of the present invention is an antibody which antagonises PD1 signalling. In an embodiment, this antibody is an anti-PDL1 antibody. In an embodiment, this antibody is an anti-PD1 antibody.

In a preferred embodiment, the anti-PD1 antibody in the pharmaceutical combination of the present invention is nivolumab (trade name OPDIVO®, available from Bristol Myers Squibb). This definition of anti-PD1 antibody used in the pharmaceutical combinations of the present invention is referred to herein as “T6” or “Therapeutic T6”.

In an embodiment, the anti-PDL1 antibody in the pharmaceutical combination of the present invention is atezolizumab (trade name Tecentriq®, available from Genentech/Roche). This definition of anti-PDL1 antibody used in the pharmaceutical combinations of the present invention is referred to herein as “T7” or “Therapeutic T7”.

The most preferred pharmaceutical combinations of the present invention which comprise an antibody which antagonises PD1 signalling comprise only one type of antibody which antagonises PD1 signalling. In an embodiment, the antibody which antagonises PD1 signalling is administered subcutaneously. In an embodiment, the antibody is T6 and is administered subcutaneously.

### Nucleotide Analogue

In an embodiment, a therapeutic used in the pharmaceutical combination of the present invention is a nucleotide analogue.

In an embodiment, the nucleotide analogue in the pharmaceutical combination of the present invention is selected from among: Lamivudine, Telbivudine, Entecavir, Adefovir, Tenofovir, Clevudine, Tenofovir alafenamide, CMX157, and AGX-1009.

In an embodiment, the nucleotide analogue in the pharmaceutical combination of the present invention is Entecavir. This definition of nucleotide analogue used in the pharmaceutical combinations of the present invention is referred to herein as “T8” or “Therapeutic T8”.

In an embodiment, the nucleotide analogue in the pharmaceutical combination of the present invention is Tenofovir. This definition of nucleotide analogue used in the pharmaceutical combinations of the present invention is referred to herein as “T9” or “Therapeutic T9.

In an embodiment, the nucleotide analogue is administered subcutaneously.

### Pharmaceutical Combinations

The present invention provides various pharmaceutical combinations comprising at least two HBV therapeutics, preferably comprising two or three HBV therapeutics.

Pharmaceutical combinations of the present invention which comprise two specified HBV therapeutics are described in Table 2 below.

Table 2: Preferred pharmaceutical combinations of the present invention which comprise two specified HBV therapeutics designated as Element A and Element B. For example, Combination C1 comprises an Element A which is the therapeutic T1 as defined above, and further comprises an Element B which is the therapeutic T2 as defined above.

| Element A | Element B | Combination |
|---|---|---|
| T1 | T2 | C1 |
| T1 | T3 | C2 |
| T1 | T4 | C3 |
| T1 | T5 | C4 |
| T1 | T6 | C5 |
| T1 | T7 | C6 |
| T1 | T8 | C7 |
| T1 | T9 | C8 |
| T2 | T3 | C9 |
| T2 | T4 | C10 |
| T2 | T5 | C11 |
| T2 | T6 | C12 |
| T2 | T7 | C13 |
| T2 | T8 | C14 |
| T2 | T9 | C15 |
| T3 | T4 | C16 |
| T3 | T5 | C17 |
| T3 | T6 | C18 |
| T3 | T7 | C19 |
| T3 | T8 | C20 |
| T3 | T9 | C21 |
| T4 | T5 | C22 |
| T4 | T6 | C23 |
| T4 | T7 | C24 |
| T4 | T8 | C25 |
| T4 | T9 | C26 |
| T5 | T6 | C27 |
| T5 | T7 | C28 |
| T5 | T8 | C29 |
| T5 | T9 | C30 |
| T6 | T7 | C31 |
| T6 | T8 | C32 |
| T6 | T9 | C33 |
| T7 | T8 | C34 |
| T7 | T9 | C35 |
| T8 | T9 | C36 |

Pharmaceutical combinations of the present invention which comprise three specified HBV therapeutics are described in Table 3 below.

Table 3: Preferred pharmaceutical combinations of the present invention which comprise three specified HBV therapeutics designated as Element A, Element B and Element C. For example, Combination C37 comprises an Element A which is the therapeutic T1 as defined above, further comprises an Element B which is the therapeutic T2 as defined above, and further comprises an Element C which is the therapeutic T3 as defined above.

| Element A | Element B | Element C | Combination |
|---|---|---|---|
| T1 | T2 | T3 | C37 |
| T1 | T2 | T4 | C38 |
| T1 | T2 | T5 | C39 |
| T1 | T2 | T6 | C40 |
| T1 | T2 | T7 | C41 |
| T1 | T2 | T8 | C42 |
| T1 | T2 | T9 | C43 |
| T1 | T3 | T4 | C44 |
| T1 | T3 | T5 | C45 |
| T1 | T3 | T6 | C46 |
| T1 | T3 | T7 | C47 |
| T1 | T3 | T8 | C48 |
| T1 | T3 | T9 | C49 |
| T1 | T4 | T5 | C50 |
| T1 | T4 | T6 | C51 |
| T1 | T4 | T7 | C52 |
| T1 | T4 | T8 | C53 |
| T1 | T4 | T9 | C54 |
| T1 | T5 | T6 | C55 |
| T1 | T5 | T7 | C56 |
| T1 | T5 | T8 | C57 |
| T1 | T5 | T9 | C58 |
| T1 | T6 | T7 | C59 |
| T1 | T6 | T8 | C60 |
| T1 | T6 | T9 | C61 |
| T1 | T7 | T8 | C62 |
| T1 | T7 | T9 | C63 |
| T1 | T8 | T9 | C64 |
| T2 | T3 | T4 | C65 |
| T2 | T3 | T5 | C66 |
| T2 | T3 | T6 | C67 |
| T2 | T3 | T7 | C68 |
| T2 | T3 | T8 | C69 |
| T2 | T3 | T9 | C70 |
| T2 | T4 | T5 | C71 |
| T2 | T4 | T6 | C72 |
| T2 | T4 | T7 | C73 |
| T2 | T4 | T8 | C74 |
| T2 | T4 | T9 | C75 |
| T2 | T5 | T6 | C76 |
| T2 | T5 | T7 | C77 |
| T2 | T5 | T8 | C78 |
| T2 | T5 | T9 | C79 |
| T2 | T6 | T7 | C80 |
| T2 | T6 | T8 | C81 |
| T2 | T6 | T9 | C82 |
| T2 | T7 | T8 | C83 |
| T2 | T7 | T9 | C84 |
| T2 | T8 | T9 | C85 |
| T3 | T4 | T5 | C86 |
| T3 | T4 | T6 | C87 |
| T3 | T4 | T7 | C88 |
| T3 | T4 | T8 | C89 |
| T3 | T4 | T9 | C90 |
| T3 | T5 | T6 | C91 |
| T3 | T5 | T7 | C92 |
| T3 | T5 | T8 | C93 |
| T3 | T5 | T9 | C94 |
| T3 | T6 | T7 | C95 |
| T3 | T6 | T8 | C96 |
| T3 | T6 | T9 | C97 |
| T3 | T7 | T8 | C98 |
| T3 | T7 | T9 | C99 |
| T3 | T8 | T9 | C100 |
| T4 | T5 | T6 | C101 |
| T4 | T5 | T7 | C102 |
| T4 | T5 | T8 | C103 |
| T4 | T5 | T9 | C104 |
| T4 | T6 | T7 | C105 |
| T4 | T6 | T8 | C106 |
| T4 | T6 | T9 | C107 |
| T4 | T7 | T8 | C108 |
| T4 | T7 | T9 | C109 |
| T4 | T8 | T9 | C110 |
| T5 | T6 | T7 | C111 |
| T5 | T6 | T8 | C112 |
| T5 | T6 | T9 | C113 |
| T5 | T7 | T8 | C114 |
| T5 | T7 | T9 | C115 |
| T5 | T8 | T9 | C116 |
| T6 | T7 | T8 | C117 |
| T6 | T7 | T9 | C118 |
| T6 | T8 | T9 | C119 |
| T7 | T8 | T9 | C120 |

Now that the pharmaceutical combinations of the present invention have been described, certain preferred embodiments of the pharmaceutical combinations of the present invention are set forth herein. In a preferred embodiment, the pharmaceutical combinations of the present invention are those wherein the same combination does not comprise both Therapeutic T6 and Therapeutic T7. In a preferred embodiment, the pharmaceutical combinations of the present invention are those which comprise Therapeutic T1 in combination with one or more further HBV therapeutics. In a preferred embodiment, the pharmaceutical combinations of the present invention are those which comprise Therapeutic T1 and Therapeutic T2, optionally in combination with a further, third HBV therapeutic, preferably any one of T3, T4, T5, T6, T7, T8 or T9. In a preferred embodiment, the pharmaceutical combination of the present invention comprises T1 and T2, optionally in combination with T3.

Typically, the combinations above comprise the recited Elements, i.e. they include the stated HBV therapeutics but do not exclude the inclusion of further, unrecited HBV therapeutics. However, in another embodiment, the combinations defined above are limited to the recited elements, i.e. the pharmaceutical combinations consist essentially of the recited elements to the exclusion of any other HBV therapeutics. This does not preclude the presence of any carrier, excipient, adjuvant, diluent or salt in the combination. Therefore, in another embodiment, a pharmaceutical combination of the present invention consists essentially of the relevant Elements recited for that combination in Table 2 or 3.

In a preferred embodiment, each of the HBV therapeutics in the pharmaceutical combination of the present invention is formulated in a pharmaceutically acceptable carrier. More preferably, each HBV therapeutic is formulated in a pharmaceutically acceptable carrier which is suitable for the administration of the HBV therapeutic in question.

The pharmaceutical combinations of the present invention can be used to treat an HBV infection more effectively than the comprised individual HBV therapeutics alone. In an embodiment, the pharmaceutical combination of the present invention can be used to inhibit HBV more rapidly, to inhibit HBV with an increased duration and/or to inhibit HBV with greater effect than the comprised individual HBV therapeutics alone. These effects may be measured by a reduction in HBsAg, HBeAg or HBV-DNA titre. In an embodiment, the pharmaceutical combination of the present invention causes a more rapid reduction in HBsAg, HBeAg or HBV-DNA titre than the comprised individual HBV therapeutics alone. In an embodiment, the pharmaceutical combination of the present invention causes a more prolonged reduction in HBsAg, HBeAg or HBV-DNA titre than the comprised individual HBV therapeutics alone. In an embodiment, the pharmaceutical combination of the present invention causes a greater decrease in HBsAg, HBeAg or HBV-DNA titre than the comprised individual HBV therapeutics alone. Primarily, HBsAg is measured for this purpose.

The pharmaceutical combinations of the present invention may also be present in a kit or kit of parts. The term “kit” or “kit of parts” refers to an assembly of materials that are used in performing the treatment of an HBV infected individual, including a description of how to conduct the treatment.

An aspect of the invention is a kit of parts containing two or a plurality of therapeutically effective components (such as medical components or medicaments) selected from the HBV therapeutics as described herein.

One embodiment of the invention is a kit of parts comprising a first HBV therapeutic as described herein and a second HBV therapeutic as described herein, optionally further comprising a third HBV therapeutic as described herein, as medical components.

In one embodiment the kit of the invention contains a first medicament which is an RNAi oligonucleotide targeting HBV formulated for subcutaneous injection and a second medicament which is an anti-PDL1 antisense oligonucleotide also formulated for subcutaneous administration. The RNAi oligonucleotide targeting HBV and the anti-PDL1 antisense oligonucleotide are formulated separately. Each of the RNAi oligonucleotide targeting HBV and the anti-PDL1 antisense oligonucleotide can be formulated as a liquid in a vial with one or multiple doses or in a prefilled syringe with one pharmaceutically effective dose. Alternatively, each of the RNAi oligonucleotide targeting HBV and the anti-PDL1 antisense oligonucleotide can be in the form of lyophilized powder and the kit contains dissolvent for preparation for injection. It is understood that all medicaments for injection are sterile. If a TLR7 agonist is included in the kit, it can be in tablet form (or alternative unit dose forms for oral administrations such as capsules and gels) with a single pharmaceutically effective dose pr. tablet, the kit can contain multiple tablets.

In a further embodiment the kit of parts of the present invention further comprises instructions for administration of RNAi oligonucleotide targeting HBV in combination with an anti-PDL1 antisense oligonucleotide to treat a hepatitis B virus infection. In particular, the instructions describe the treatment of a chronic hepatitis B virus infection.

The kit may contain just one of the medical components and instructions for its use in combination with the other medical component. In one embodiment the kit of parts of the invention comprises or contains a first medicament which is an RNAi oligonucleotide targeting HBV and instructions for its use in combination with an anti-PDL1 antisense oligonucleotide as the second medicament, but which is purchased separately. In another embodiment the kit of parts of the invention comprises or contains a first medicament which is an anti-PDL1 antisense oligonucleotide and instructions for its use in combination with an RNAi oligonucleotide targeting HBV as the second medicament, but which is purchased separately.

In some embodiments the pharmaceutical combination of the invention may be used in combination with a third or further therapeutic agent(s), which may be included in the kits of part or supplied separately. In an embodiment, the further therapeutic agent is any of T3, T4, T5, T6, T7, T8 or T9. Preferably, the further therapeutic agent is T3.

### Orders of Administration for Pharmaceutical Combinations

Specific orders of HBV therapeutic administration within the pharmaceutical combinations of the invention as defined above (Combinations C1-C120) are set forth in this section.

It is to be noted that the “Element” notation used above (Element A, Element B and Element C) is purely for reference only and does not imply anything regarding the order in which the therapeutics in a particular pharmaceutical combination are to be administered. Rather, the orders of administration of the therapeutics in the pharmaceutical combinations of the present invention are explicitly set forth herein in respect of which Elements are administered first and second (and third, where relevant).

For example, in an embodiment in respect of the pharmaceutical combination “C1” of the present invention, Element A is administered first and Element B is administered second. In this embodiment of combination C1, the first or initial dose of Element A of Combination C1 (which is the HBV therapeutic defined herein as T1) is administered before the first or initial dose of Element B of combination C1 (which is the HBV therapeutic defined herein as T2). Herein, the specified orders in respect of the administration of the Elements of a specific pharmaceutical combination of the present invention are relative only to the Elements that are explicitly part of that pharmaceutical combination. For example, an Element specified as being “administered first” does not necessarily preclude that the patient has never previously been administered a different HBV therapeutic which was not administered as part of the administration of a pharmaceutical combination of the present invention.

Herein, it should also be understood that the Elements of the pharmaceutical combinations of the present invention can be administered at a single time point, e.g. as a single dose, or as multiple doses given over a period of time. Thus, a reference to an Element being “administered” herein can refer either to the specific time where that Element is administered (with a dose given at a single time point), or to the time where the administering of the Element is initiated (with doses given over a period of time). For this reason, it is envisaged that pharmaceutical combinations of the invention may involve overlapping dosage regimes. For example, in a pharmaceutical combination of the invention wherein an Element A is administered first and an Element B is administered second, when Element A is administered as several doses over a period of time, the administration of further doses of Element A may overlap with the administration of Element B, provided that the initial dose of Element A is administered before the single or initial dose of Element B.

### Combination C1

In an embodiment of the present invention, the pharmaceutical combination is combination C1 comprising the Element A and Element B as defined in Table 2 above. In an embodiment of this combination, Element A is administered prior to the administering of Element B. In a further embodiment of this combination, Element B is administered prior to the administering of Element A.

### Combination C2

In an embodiment of the present invention, the pharmaceutical combination is combination C2 comprising the Element A and Element B as defined in Table 2 above. In an embodiment of this combination, Element A is administered prior to the administering of Element B. In a further embodiment of this combination, Element B is administered prior to the administering of Element A.

### Combination C3

In an embodiment of the present invention, the pharmaceutical combination is combination C3 comprising the Element A and Element B as defined in Table 2 above. In an embodiment of this combination, Element A is administered prior to the administering of Element B. In a further embodiment of this combination, Element B is administered prior to the administering of Element A.

### Combination C4

In an embodiment of the present invention, the pharmaceutical combination is combination C4 comprising the Element A and Element B as defined in Table 2 above. In an embodiment of this combination, Element A is administered prior to the administering of Element B. In a further embodiment of this combination, Element B is administered prior to the administering of Element A.

### Combination C5

In an embodiment of the present invention, the pharmaceutical combination is combination C5 comprising the Element A and Element B as defined in Table 2 above. In an embodiment of this combination, Element A is administered prior to the administering of Element B. In a further embodiment of this combination, Element B is administered prior to the administering of Element A.

### Combination C6

In an embodiment of the present invention, the pharmaceutical combination is combination C6 comprising the Element A and Element B as defined in Table 2 above. In an embodiment of this combination, Element A is administered prior to the administering of Element B. In a further embodiment of this combination, Element B is administered prior to the administering of Element A.

### Combination C7

In an embodiment of the present invention, the pharmaceutical combination is combination C7 comprising the Element A and Element B as defined in Table 2 above. In an embodiment of this combination, Element A is administered prior to the administering of Element B. In a further embodiment of this combination, Element B is administered prior to the administering of Element A.

### Combination C8

In an embodiment of the present invention, the pharmaceutical combination is combination C8 comprising the Element A and Element B as defined in Table 2 above. In an embodiment of this combination, Element A is administered prior to the administering of Element B. In a further embodiment of this combination, Element B is administered prior to the administering of Element A.

### Combination C9

In an embodiment of the present invention, the pharmaceutical combination is combination C9 comprising the Element A and Element B as defined in Table 2 above. In an embodiment of this combination, Element A is administered prior to the administering of Element B. In a further embodiment of this combination, Element B is administered prior to the administering of Element A.

### Combination C10

In an embodiment of the present invention, the pharmaceutical combination is combination C10 comprising the Element A and Element B as defined in Table 2 above. In an embodiment of this combination, Element A is administered prior to the administering of Element B. In a further embodiment of this combination, Element B is administered prior to the administering of Element A.

### Combination C11

In an embodiment of the present invention, the pharmaceutical combination is combination C11 comprising the Element A and Element B as defined in Table 2 above. In an embodiment of this combination, Element A is administered prior to the administering of Element B. In a further embodiment of this combination, Element B is administered prior to the administering of Element A.

### Combination C12

In an embodiment of the present invention, the pharmaceutical combination is combination C12 comprising the Element A and Element B as defined in Table 2 above. In an embodiment of this combination, Element A is administered prior to the administering of Element B. In a further embodiment of this combination, Element B is administered prior to the administering of Element A.

### Combination C13

In an embodiment of the present invention, the pharmaceutical combination is combination C13 comprising the Element A and Element B as defined in Table 2 above. In an embodiment of this combination, Element A is administered prior to the administering of Element B. In a further embodiment of this combination, Element B is administered prior to the administering of Element A.

### Combination C14

In an embodiment of the present invention, the pharmaceutical combination is combination C14 comprising the Element A and Element B as defined in Table 2 above. In an embodiment of this combination, Element A is administered prior to the administering of Element B. In a further embodiment of this combination, Element B is administered prior to the administering of Element A.

### Combination C15

In an embodiment of the present invention, the pharmaceutical combination is combination C15 comprising the Element A and Element B as defined in Table 2 above. In an embodiment of this combination, Element A is administered prior to the administering of Element B. In a further embodiment of this combination, Element B is administered prior to the administering of Element A.

### Combination C16

In an embodiment of the present invention, the pharmaceutical combination is combination C16 comprising the Element A and Element B as defined in Table 2 above. In an embodiment of this combination, Element A is administered prior to the administering of Element B. In a further embodiment of this combination, Element B is administered prior to the administering of Element A.

### Combination C17

In an embodiment of the present invention, the pharmaceutical combination is combination C17 comprising the Element A and Element B as defined in Table 2 above. In an embodiment of this combination, Element A is administered prior to the administering of Element B. In a further embodiment of this combination, Element B is administered prior to the administering of Element A.

### Combination C18

In an embodiment of the present invention, the pharmaceutical combination is combination C18 comprising the Element A and Element B as defined in Table 2 above. In an embodiment of this combination, Element A is administered prior to the administering of Element B. In a further embodiment of this combination, Element B is administered prior to the administering of Element A.

### Combination C19

In an embodiment of the present invention, the pharmaceutical combination is combination C19 comprising the Element A and Element B as defined in Table 2 above. In an embodiment of this combination, Element A is administered prior to the administering of Element B. In a further embodiment of this combination, Element B is administered prior to the administering of Element A.

### Combination C20

In an embodiment of the present invention, the pharmaceutical combination is combination C20 comprising the Element A and Element B as defined in Table 2 above. In an embodiment of this combination, Element A is administered prior to the administering of Element B. In a further embodiment of this combination, Element B is administered prior to the administering of Element A.

### Combination C21

In an embodiment of the present invention, the pharmaceutical combination is combination C21 comprising the Element A and Element B as defined in Table 2 above. In an embodiment of this combination, Element A is administered prior to the administering of Element B. In a further embodiment of this combination, Element B is administered prior to the administering of Element A.

### Combination C22

In an embodiment of the present invention, the pharmaceutical combination is combination C22 comprising the Element A and Element B as defined in Table 2 above. In an embodiment of this combination, Element A is administered prior to the administering of Element B. In a further embodiment of this combination, Element B is administered prior to the administering of Element A.

### Combination C23

In an embodiment of the present invention, the pharmaceutical combination is combination C23 comprising the Element A and Element B as defined in Table 2 above. In an embodiment of this combination, Element A is administered prior to the administering of Element B. In a further embodiment of this combination, Element B is administered prior to the administering of Element A.

### Combination C24

In an embodiment of the present invention, the pharmaceutical combination is combination C24 comprising the Element A and Element B as defined in Table 2 above. In an embodiment of this combination, Element A is administered prior to the administering of Element B. In a further embodiment of this combination, Element B is administered prior to the administering of Element A.

### Combination C25

In an embodiment of the present invention, the pharmaceutical combination is combination C25 comprising the Element A and Element B as defined in Table 2 above. In an embodiment of this combination, Element A is administered prior to the administering of Element B. In a further embodiment of this combination, Element B is administered prior to the administering of Element A.

### Combination C26

In an embodiment of the present invention, the pharmaceutical combination is combination C26 comprising the Element A and Element B as defined in Table 2 above. In an embodiment of this combination, Element A is administered prior to the administering of Element B. In a further embodiment of this combination, Element B is administered prior to the administering of Element A.

### Combination C27

In an embodiment of the present invention, the pharmaceutical combination is combination C27 comprising the Element A and Element B as defined in Table 2 above. In an embodiment of this combination, Element A is administered prior to the administering of Element B. In a further embodiment of this combination, Element B is administered prior to the administering of Element A.

### Combination C28

In an embodiment of the present invention, the pharmaceutical combination is combination C28 comprising the Element A and Element B as defined in Table 2 above. In an embodiment of this combination, Element A is administered prior to the administering of Element B. In a further embodiment of this combination, Element B is administered prior to the administering of Element A.

### Combination C29

In an embodiment of the present invention, the pharmaceutical combination is combination C29 comprising the Element A and Element B as defined in Table 2 above. In an embodiment of this combination, Element A is administered prior to the administering of Element B. In a further embodiment of this combination, Element B is administered prior to the administering of Element A.

### Combination C30

In an embodiment of the present invention, the pharmaceutical combination is combination C30 comprising the Element A and Element B as defined in Table 2 above. In an embodiment of this combination, Element A is administered prior to the administering of Element B. In a further embodiment of this combination, Element B is administered prior to the administering of Element A.

### Combination C31

In an embodiment of the present invention, the pharmaceutical combination is combination C31 comprising the Element A and Element B as defined in Table 2 above. In an embodiment of this combination, Element A is administered prior to the administering of Element B. In a further embodiment of this combination, Element B is administered prior to the administering of Element A.

### Combination C32

In an embodiment of the present invention, the pharmaceutical combination is combination C32 comprising the Element A and Element B as defined in Table 2 above. In an embodiment of this combination, Element A is administered prior to the administering of Element B. In a further embodiment of this combination, Element B is administered prior to the administering of Element A.

### Combination C33

In an embodiment of the present invention, the pharmaceutical combination is combination C33 comprising the Element A and Element B as defined in Table 2 above. In an embodiment of this combination, Element A is administered prior to the administering of Element B. In a further embodiment of this combination, Element B is administered prior to the administering of Element A.

### Combination C34

In an embodiment of the present invention, the pharmaceutical combination is combination C34 comprising the Element A and Element B as defined in Table 2 above. In an embodiment of this combination, Element A is administered prior to the administering of Element B. In a further embodiment of this combination, Element B is administered prior to the administering of Element A.

### Combination C35

In an embodiment of the present invention, the pharmaceutical combination is combination C35 comprising the Element A and Element B as defined in Table 2 above. In an embodiment of this combination, Element A is administered prior to the administering of Element B. In a further embodiment of this combination, Element B is administered prior to the administering of Element A.

### Combination C36

In an embodiment of the present invention, the pharmaceutical combination is combination C36 comprising the Element A and Element B as defined in Table 2 above. In an embodiment of this combination, Element A is administered prior to the administering of Element B. In a further embodiment of this combination, Element B is administered prior to the administering of Element A.

### Combination C37

In an embodiment of the present invention, the pharmaceutical combination is combination C37 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C38

In an embodiment of the present invention, the pharmaceutical combination is combination C38 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C39

In an embodiment of the present invention, the pharmaceutical combination is combination C39 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C40

In an embodiment of the present invention, the pharmaceutical combination is combination C40 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C41

In an embodiment of the present invention, the pharmaceutical combination is combination C41 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C42

In an embodiment of the present invention, the pharmaceutical combination is combination C42 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C43

In an embodiment of the present invention, the pharmaceutical combination is combination C43 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C44

In an embodiment of the present invention, the pharmaceutical combination is combination C44 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C45

In an embodiment of the present invention, the pharmaceutical combination is combination C45 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C46

In an embodiment of the present invention, the pharmaceutical combination is combination C46 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C47

In an embodiment of the present invention, the pharmaceutical combination is combination C47 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C48

In an embodiment of the present invention, the pharmaceutical combination is combination C48 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C49

In an embodiment of the present invention, the pharmaceutical combination is combination C49 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C50

In an embodiment of the present invention, the pharmaceutical combination is combination C50 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C51

In an embodiment of the present invention, the pharmaceutical combination is combination C51 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C52

In an embodiment of the present invention, the pharmaceutical combination is combination C52 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C53

In an embodiment of the present invention, the pharmaceutical combination is combination C53 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C54

In an embodiment of the present invention, the pharmaceutical combination is combination C54 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C55

In an embodiment of the present invention, the pharmaceutical combination is combination C55 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C56

In an embodiment of the present invention, the pharmaceutical combination is combination C56 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C57

In an embodiment of the present invention, the pharmaceutical combination is combination C57 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C58

In an embodiment of the present invention, the pharmaceutical combination is combination C58 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C59

In an embodiment of the present invention, the pharmaceutical combination is combination C59 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C60

In an embodiment of the present invention, the pharmaceutical combination is combination C60 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C61

In an embodiment of the present invention, the pharmaceutical combination is combination C61 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C62

In an embodiment of the present invention, the pharmaceutical combination is combination C62 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C63

In an embodiment of the present invention, the pharmaceutical combination is combination C63 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C64

In an embodiment of the present invention, the pharmaceutical combination is combination C64 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C65

In an embodiment of the present invention, the pharmaceutical combination is combination C65 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C66

In an embodiment of the present invention, the pharmaceutical combination is combination C66 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C67

In an embodiment of the present invention, the pharmaceutical combination is combination C67 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C68

In an embodiment of the present invention, the pharmaceutical combination is combination C68 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C69

In an embodiment of the present invention, the pharmaceutical combination is combination C69 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C70

In an embodiment of the present invention, the pharmaceutical combination is combination C70 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C71

In an embodiment of the present invention, the pharmaceutical combination is combination C71 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C72

In an embodiment of the present invention, the pharmaceutical combination is combination C72 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C73

In an embodiment of the present invention, the pharmaceutical combination is combination C73 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C74

In an embodiment of the present invention, the pharmaceutical combination is combination C74 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C75

In an embodiment of the present invention, the pharmaceutical combination is combination C75 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C76

In an embodiment of the present invention, the pharmaceutical combination is combination C76 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C77

In an embodiment of the present invention, the pharmaceutical combination is combination C77 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C78

In an embodiment of the present invention, the pharmaceutical combination is combination C78 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C79

In an embodiment of the present invention, the pharmaceutical combination is combination C79 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C80

In an embodiment of the present invention, the pharmaceutical combination is combination C80 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C81

In an embodiment of the present invention, the pharmaceutical combination is combination C81 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C82

In an embodiment of the present invention, the pharmaceutical combination is combination C82 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C83

In an embodiment of the present invention, the pharmaceutical combination is combination C83 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C84

In an embodiment of the present invention, the pharmaceutical combination is combination C84 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C85

In an embodiment of the present invention, the pharmaceutical combination is combination C85 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C86

In an embodiment of the present invention, the pharmaceutical combination is combination C86 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C87

In an embodiment of the present invention, the pharmaceutical combination is combination C87 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C88

In an embodiment of the present invention, the pharmaceutical combination is combination C88 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C89

In an embodiment of the present invention, the pharmaceutical combination is combination C89 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C90

In an embodiment of the present invention, the pharmaceutical combination is combination C90 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C91

In an embodiment of the present invention, the pharmaceutical combination is combination C91 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C92

In an embodiment of the present invention, the pharmaceutical combination is combination C92 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C93

In an embodiment of the present invention, the pharmaceutical combination is combination C93 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C94

In an embodiment of the present invention, the pharmaceutical combination is combination C94 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C95

In an embodiment of the present invention, the pharmaceutical combination is combination C95 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C96

In an embodiment of the present invention, the pharmaceutical combination is combination C96 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C97

In an embodiment of the present invention, the pharmaceutical combination is combination C97 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C98

In an embodiment of the present invention, the pharmaceutical combination is combination C98 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C99

In an embodiment of the present invention, the pharmaceutical combination is combination C99 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C100

In an embodiment of the present invention, the pharmaceutical combination is combination C100 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C101

In an embodiment of the present invention, the pharmaceutical combination is combination C101 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C102

In an embodiment of the present invention, the pharmaceutical combination is combination C102 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C103

In an embodiment of the present invention, the pharmaceutical combination is combination C103 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C104

In an embodiment of the present invention, the pharmaceutical combination is combination C104 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C105

In an embodiment of the present invention, the pharmaceutical combination is combination C105 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C106

In an embodiment of the present invention, the pharmaceutical combination is combination C106 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C107

In an embodiment of the present invention, the pharmaceutical combination is combination C107 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C108

In an embodiment of the present invention, the pharmaceutical combination is combination C108 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C109

In an embodiment of the present invention, the pharmaceutical combination is combination C109 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C110

In an embodiment of the present invention, the pharmaceutical combination is combination C110 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C111

In an embodiment of the present invention, the pharmaceutical combination is combination C111 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C112

In an embodiment of the present invention, the pharmaceutical combination is combination C112 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C113

In an embodiment of the present invention, the pharmaceutical combination is combination C113 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C114

In an embodiment of the present invention, the pharmaceutical combination is combination C114 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C115

In an embodiment of the present invention, the pharmaceutical combination is combination C115 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C116

In an embodiment of the present invention, the pharmaceutical combination is combination C116 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C117

In an embodiment of the present invention, the pharmaceutical combination is combination C117 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C118

In an embodiment of the present invention, the pharmaceutical combination is combination C118 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C119

In an embodiment of the present invention, the pharmaceutical combination is combination C119 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Combination C120

In an embodiment of the present invention, the pharmaceutical combination is combination C120 comprising the Element A, Element B and Element C as defined in Table 3 above. In an embodiment of this combination, Element A is administered prior to the administering of Element B, and Element B is administered prior to the administering of Element C. In a further embodiment of this combination, Element A is administered prior to the administering of Element C, and Element C is administered prior to the administering of Element B. In a further embodiment of this combination, Element B is administered prior to the administering of Element A, and Element A is administered prior to the administering of Element C. In a further embodiment of this combination, Element B is administered prior to the administering of Element C, and Element C is administered prior to the administering of Element A. In a further embodiment of this combination, Element C is administered prior to the administering of Element A, and Element A is administered prior to the administering of Element B. In a further embodiment of this combination, Element C is administered prior to the administering of Element B, and Element B is administered prior to the administering of Element A.

### Preferred Features of Administration Orders

Now that administration orders of the present invention have been described, the more preferred administration orders of the present invention are set forth. In a preferred embodiment where a pharmaceutical combination of the present invention comprises the Therapeutic T1, Therapeutic T1 is administered first. In a preferred embodiment where a pharmaceutical combination of the present invention comprises the Therapeutic T1 and the Therapeutic T2, Therapeutic T1 is administered prior to the administering of Therapeutic T2, preferably at least a week (seven days) prior to the administering of Therapeutic T2, more preferably at least four weeks prior to the administering of Therapeutic T2. In a preferred embodiment where a pharmaceutical combination of the present invention comprises the Therapeutic T1, the Therapeutic T2 and an additional third HBV therapeutic, Therapeutic T1 is administered prior to the administering of Therapeutic T2 and the additional third HBV therapeutic, preferably at least a week prior to the administering of Therapeutic T2 and the additional third HBV therapeutic.

In a preferred embodiment of administering any of combinations C1, C2, C3, C4, C5, C6, C7, C8, C37, C38, C39, C40, C41, C42, C43, C44, C45, C46, C47, C48, C49, C50, C51, C52, C53, C54, C55, C56, C57, C58, C59, C60, C61, C62, C63 or C64, Therapeutic T1 is administered first, preferably at least a week prior to the administration of the other comprised HBV therapeutics. In a preferred embodiment of administering any of combinations C1, C37, C38, C39, C40, C41, C42 or C43, Therapeutic T1 is administered prior to the administering of Therapeutic T2, preferably at least a week prior to the administering of Therapeutic T2, more preferably at least four weeks prior to the administering of Therapeutic T2. In a preferred embodiment of administering any of combinations C37, C38, C39, C40, C41, C42 or C43, Therapeutic T1 is administered prior to the administering of Therapeutic T2 and the additional HBV therapeutic, preferably at least a week prior to the administering of Therapeutic T2 and the additional HBV therapeutic.

### Particularly Advantageous Combination and Dosage Thereof

A most preferred pharmaceutical combination of the present invention comprises an RNAi oligonucleotide targeting HBV as defined herein and an anti-PDL1 antisense oligonucleotide as defined herein. The present inventors have unexpectedly found advantageous and synergistic effects between these HBV therapeutics when used in a pharmaceutical combination. When used in combination, the RNAi oligonucleotide targeting HBV and the anti-PDL1 antisense oligonucleotide defined herein are capable of achieving a reduction in HBV viral markers (serum HBsAg, HBeAg and HBV-DNA) which is greater than the reduction achieved by either HBV therapeutic as a monotherapy alone, and is even greater than the sum of both the effects achieved by each HBV therapeutic as a monotherapy.

This combination optionally comprises a further, different, third HBV therapeutic as described herein. In an embodiment, the third HBV therapeutic is selected from a TLR7 agonist, interferon-alpha, an anti-HBV antibody, an anti-PDL1 antibody or a nucleotide analogue as defined herein. In a preferred embodiment, the third HBV therapeutic is a TLR7 agonist, preferably the TLR7 agonist defined as T3 herein. In a preferred embodiment, the dosage of the TLR7 agonist in this combination is according to the dosage disclosed for TLR7 agonists in the corresponding section above.

In a general embodiment in the context of this pharmaceutical combination, the anti-PDL1 antisense oligonucleotide is administered in one or more doses of at least about 0.1 mg/kg, preferably of at least about 1 mg/kg, preferably at least about 2 mg/kg, preferably at least about 3 mg/kg. In an embodiment, the doses are more than 3 mg/kg. In a more specific embodiment, the anti-PDL1 antisense oligonucleotide is administered in one or more doses of about 0.1 mg/kg to about 35 mg/kg, preferably about 1 mg/kg to about 35 mg/kg, preferably about 2 mg/kg to about 35 mg/kg, preferably about 3 mg/kg to about 35 mg/kg, preferably about 7 mg/kg to about 35 mg/kg.

In an embodiment, this combination results in continuously and significantly decreased serum levels of HBsAg, relative to a vehicle control. In an embodiment, this combination provides a reduction in serum HBsAg which is greater than the reduction provided by an equivalent monotherapy with the RNAi oligonucleotide targeting HBV or anti-PD-L1 antisense oligonucleotide alone. In an embodiment, the reduction is greater than the sum of these equivalent monotherapies.

In an embodiment, this combination results in continuously and significantly decreased serum levels of HBeAg, relative to a vehicle control. In an embodiment, this combination provides a reduction in serum HBeAg which is greater than the reduction provided by an equivalent monotherapy with the RNAi oligonucleotide targeting HBV or anti-PD-L1 antisense oligonucleotide alone. In an embodiment, the reduction is greater than the sum of these equivalent monotherapies.

In an embodiment, this combination results in continuously and significantly decreased serum levels of HBV-DNA, relative to a vehicle control. In an embodiment, this combination provides a reduction in serum HBV-DNA which is greater than the reduction provided by an equivalent monotherapy with the RNAi oligonucleotide targeting HBV or anti-PD-L1 antisense oligonucleotide alone. In an embodiment, the reduction is greater than the sum of these equivalent monotherapies.

In an embodiment, this combination results in continuously and significantly decreased serum levels of HBsAg, HBeAg and HBV-DNA, relative to a vehicle control. In an embodiment, reduction in serum HBsAg, HBV-DNA and HBeAg is greater than the reduction provided by an equivalent monotherapy with the RNAi oligonucleotide targeting HBV or anti-PD-L1 antisense oligonucleotide alone. In an embodiment, the reduction is greater than the sum of these equivalent monotherapies

By an equivalent monotherapy alone, it is meant that the pharmaceutical combination of the present invention obtains a greater reduction in HBV serum marker over the same dose of either of the same medicaments as are comprised in the combination. By the sum of the equivalent monotherapies, it is meant that the pharmaceutical combination obtains an improved reduction in HBV serum markers which is greater than the sum of the reductions that would be seen when administering each of the medicaments comprised therein—the RNAi oligonucleotide and anti-PDL1 oligonucleotide—as a monotherapy. The reduction in HBV serum markers including HBsAg, HBeAg and HBV-DNA is seen in the serum of the patient to which the pharmaceutical combination is administered.

In an embodiment, the pharmaceutical combination comprising an RNAi oligonucleotide targeting HBV as defined herein and an anti-PDL1 antisense oligonucleotide as defined herein provides a synergistic effect on reducing HBV serum viral markers, preferably on one or more or all of HBsAg, HBeAg and HBV-DNA. The synergistic effect obtained by this combination is unexpectedly greater than the sum of the individual effects of 1) the RNAi oligonucleotide targeting HBV and 2) the anti-PDL1 antisense oligonucleotide, i.e. when they are each administered as an equivalent mono-therapy.

In an embodiment, the RNAi oligonucleotide targeting HBV is administered first. In an embodiment, the initial or single dose of the RNAi oligonucleotide targeting HBV is administered prior to the initial or single dose of the anti-PDL1 antisense oligonucleotide.

In an embodiment, the RNAi oligonucleotide targeting HBV is administered in one or more doses of at least about 3 mg/kg, preferably more than 3 mg/kg, preferably at least about 6 mg/kg, preferably at least about 9 mg/kg.

In an embodiment, the anti-PDL1 antisense oligonucleotide is administered in one or more doses of at least about 3 mg/kg, preferably more than 3 mg/kg, preferably at least about 6 mg/kg. In an embodiment, the RNAi oligonucleotide targeting HBV is administered first and the anti-PDL1 antisense oligonucleotide is administered second. In an embodiment, the RNAi oligonucleotide targeting HBV is administered first at a single dose (DO of treatment), prior to the administering of the anti-PDL1 antisense oligonucleotide once weekly, or once every two weeks, in at least two or more doses. In an embodiment, the single or initial dose of the RNAi oligonucleotide targeting HBV is administered at least about a week, at least about two weeks, at least about three weeks, at least about four weeks, at least about five weeks, at least about six weeks, at least about seven weeks, at least about eight weeks or longer than eight weeks prior to the administering of the initial dose of the anti-PDL1 antisense oligonucleotide. In an embodiment of this dosage regime, the RNAi oligonucleotide targeting HBV is administered at a dose of between 3 mg/kg and 9 mg/kg, and the anti-PDL1 antisense oligonucleotide is administered at doses from about 3 mg/kg to about 6 mg/kg.

In an embodiment, the RNAi oligonucleotide targeting HBV is administered at a single dose of between 3 and 9 mg/kg, prior to the administering of the anti-PDL1 antisense oligonucleotide, once weekly, in at least 5 doses of between 3 and 6 mg/kg. Preferably, the initial dose of the RNAi oligonucleotide targeting HBV is administered at least 7 days, preferably at least a month, prior to the dose of the anti-PDL1 antisense oligonucleotide.

In a highly preferred embodiment, two or more, preferably at least five, doses of antiPDL1 antisense oligonucleotide are administered, once weekly or once every two weeks, with the single or initial dose of the RNAi oligonucleotide targeting HBV being administered at least about 7 days prior to the initial dose of anti-PDL1 antisense oligonucleotide.

In a highly preferred embodiment, the dose of the RNAi oligonucleotide targeting HBV is more than 3 mg/kg, preferably at least about 9 mg/kg.

In a highly preferred embodiment, the dose of the anti-PDL1 antisense oligonucleotide is more than 3 mg/kg, preferably at least about 6 mg/kg.

In a highly preferred embodiment, two or more, preferably at least five, doses of anti-PDL1 antisense oligonucleotide are administered, once weekly or once every two weeks, with the single or initial dose of the RNAi oligonucleotide targeting HBV being administered at least about 7 days prior to the initial dose of anti-PDL1 antisense oligonucleotide; and the doses of the anti-PDL1 antisense oligonucleotide are more than 3 mg/kg, preferably at least about 6 mg/kg.

In a highly preferred embodiment, two or more, preferably at least five, doses of anti-PDL1 antisense oligonucleotide are administered, once weekly, with the single or initial dose of the RNAi oligonucleotide targeting HBV being administered at least about 7 days prior to the initial dose of anti-PDL1 antisense oligonucleotide; and the dose of the RNAi oligonucleotide targeting HBV is more than 3 mg/kg, preferably at least about 9 mg/kg.

In a most preferred embodiment, two or more, preferably at least five, doses of anti-PDL1 antisense oligonucleotide are administered, once weekly, with the single or initial dose of the RNAi oligonucleotide targeting HBV being administered at least about 7 days prior to the initial dose of anti-PDL1 antisense oligonucleotide; and the dose of the RNAi oligonucleotide targeting HBV is more than 3 mg/kg, preferably at least about 9 mg/kg; and each dose of the anti-PDL1 antisense oligonucleotide is more than 3 mg/kg, preferably at least about 6 mg/kg.

### Pharmaceutical Compositions

In a further aspect, the invention provides pharmaceutical compositions comprising each of the RNAi oligonucleotide targeting HBV and the anti-PDL1 antisense oligonucleotide described herein and a pharmaceutically acceptable excipient, diluent, carrier, salt and/or adjuvant. In an embodiment, the RNAi oligonucleotide targeting HBV and the anti-PDL1 antisense oligonucleotide in the pharmaceutical combination of the present invention are present in separate compositions. In an embodiment, the therapeutic oligonucleotides are each formulated in phosphate buffered saline for subcutaneous administration.

A therapeutic oligonucleotide in the pharmaceutical combination of the invention may be mixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered. A pharmaceutically acceptable diluent of therapeutic oligonucleotides includes phosphate-buffered saline (PBS) and pharmaceutically acceptable salts include, but are not limited to, sodium and potassium salts. In some embodiments the pharmaceutically acceptable diluent of the therapeutic oligonucleotide is sterile phosphate buffered saline. In some embodiments the oligonucleotide is used in the pharmaceutically acceptable diluent at a concentration of 50-150 mg/ml solution. The therapeutic oligonucleotide or pharmaceutical composition comprising the therapeutic oligonucleotide is administered by a parenteral route including intravenous, intraarterial, subcutaneous or intramuscular injection or infusion. In one embodiment the oligonucleotide conjugate is administered intravenously. For therapeutic oligonucleotides it is advantageous if they are administered subcutaneously.

These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents, such as in a sealed package of tablets or capsules.

### Formulations of Therapeutic Oligonucleotides

Various formulations have been developed to facilitate therapeutic oligonucleotide use, which may be applicable to therapeutic oligonucleotides used in the pharmaceutical combinations of the present invention. For example, oligonucleotides can be delivered to a subject or a cellular environment using a formulation that minimizes degradation, facilitates delivery and/or uptake, or provides another beneficial property to the oligonucleotides in the formulation. In some embodiments, provided herein are pharmaceutical combinations comprising a first medicament which is a composition comprising an oligonucleotide (e.g., a single-stranded or double-stranded oligonucleotide) to reduce the expression of HBV antigen (e.g., HBsAg). Such compositions can be suitably formulated such that when administered to a subject, either into the immediate environment of a target cell or systemically, a sufficient portion of the oligonucleotides enter the cell to reduce HBV antigen expression. Any of a variety of suitable oligonucleotide formulations can be used to deliver oligonucleotides for the reduction of HBV antigen as disclosed herein. In some embodiments, an oligonucleotide of the pharmaceutical combination of the present invention is formulated in buffer solutions such as phosphate-buffered saline solutions, liposomes, micellar structures, and capsids.

Formulations of oligonucleotides with cationic lipids can be used to facilitate transfection of the oligonucleotides into cells. For example, cationic lipids, such as lipofectin, cationic glycerol derivatives, and polycationic molecules (e.g., polylysine) can be used. Suitable lipids include Oligofectamine, Lipofectamine (Life Technologies), NC388 (Ribozyme Pharmaceuticals, Inc., Boulder, Colo.), or FuGene 6 (Roche) all of which can be used according to the manufacturer's instructions.

Accordingly, in some embodiments, an oligonucleotide formulation comprises a lipid nanoparticle. In some embodiments, an excipient comprises a liposome, a lipid, a lipid complex, a microsphere, a microparticle, a nanosphere, or a nanoparticle, or may be otherwise formulated for administration to the cells, tissues, organs, or body of a subject in need thereof (see, e.g., Remington: The Science and Practice of Pharmacy, 22nd edition, Pharmaceutical Press, 2013).

In some embodiments, formulations as disclosed herein comprise an excipient. In some embodiments, an excipient confers to a composition improved stability, improved absorption, improved solubility and/or therapeutic enhancement of the active ingredient. In some embodiments, an excipient is a buffering agent (e.g., sodium citrate, sodium phosphate, a tris base, or sodium hydroxide) or a vehicle (e.g., a buffered solution, petrolatum, dimethyl sulfoxide, or mineral oil). In some embodiments, an oligonucleotide is lyophilized for extending its shelf-life and then made into a solution before use (e.g., administration to a subject). Accordingly, an excipient in a composition comprising any one of the oligonucleotides described herein may be a lyoprotectant (e.g., mannitol, lactose, polyethylene glycol, or polyvinyl pyrolidone), or a collapse temperature modifier (e.g., dextran, ficoll, or gelatin).

In some embodiments of the pharmaceutical combination of the present invention, the composition comprising an oligonucleotide is formulated to be compatible with its intended route of administration. Examples of routes of administration include parenteral, e.g., intravenous, intradermal, subcutaneous, oral (e.g., inhalation), transdermal (topical), transmucosal, and rectal administration. Formulation for subcutaneous is particularly advantageous where the oligonucleotide in the pharmaceutical combination of the present invention is an RNAi oligonucleotide.

Pharmaceutical compositions suitable for injectable use include sterile aqueous solutions (where water soluble) or dispersions and sterile powders for the extemporaneous preparation of sterile injectable solutions or dispersions. Suitable carriers include physiological saline, bacteriostatic water, Cremophor EL™ (BASF, Parsippany, N.J.) or phosphate buffered saline (PBS). The carrier may be water or a solvent or dispersion medium. The solvent or dispersion medium may contain, for example, water, ethanol, polyol (for example, glycerol, propylene glycol, and liquid polyethylene glycol, and the like), and suitable mixtures thereof. In many cases, it will be preferable to include isotonic agents, for example, sugars, polyalcohols such as mannitol, sorbitol, and sodium chloride in the composition. Sterile injectable solutions can be prepared by incorporating the oligonucleotides in a required amount in a selected solvent with one or a combination of ingredients enumerated above, as required, followed by filtered sterilization.

In some embodiments of the pharmaceutical combination of the present invention, a composition in the combination may contain at least about 0.1% of the therapeutic agent (e.g., an oligonucleotide for reducing HBV antigen expression) or more, although the percentage of the active ingredient(s) may be between about 1% and about 80% or more of the weight or volume of the total composition. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

Even though a number of embodiments are directed to liver-targeted delivery of any of the oligonucleotides disclosed herein, targeting of other tissues is also contemplated.

### Applications

The pharmaceutical combination of the present invention is for use in treatment of Hepatitis B virus infections, in particular treatment of patients with chronic HBV. The pharmaceutical combination of the invention may be utilized as therapeutics and in prophylaxis.

The pharmaceutical combination of the invention can be used as a combined hepatitis B virus targeting therapy and an immunotherapy. In particular, the pharmaceutical combination of the invention is capable of affecting one or more of the following HBV infection parameters i) reducing cellular HBV mRNA, ii) reducing HBV DNA in serum and/or iii) reducing HBV viral antigens, such as HBsAg and HBeAg when used in the treatment of HBV in an infected cell. In an embodiment of the invention the effect on one or more of these parameters is improved compared to the effect achieved when performing the treatment with an individual HBV therapeutic of the pharmaceutical combination.

The effect on a HBV infection may be measured in vitro using HBV infected primary human hepatocytes or HBV infected HepaRG cells or ASGPR-HepaRG cells (see for example PCT/EP2018/078136). The effect on a HBV infection may also be measured in vivo using AAV/HBV mouse model of mice infected with a recombinant adeno-associated virus (AAV) carrying the HBV genome (AAV/HBV) (Dan Yang, et al. 2014 Cellular & Molecular Immunology 11, 71-78) or HBV minicircle mouse (available at Covance Shanghai, see also Guo et al 2016 Sci Rep 6: 2552 and Yan et al 2017 J Hepatology 66(6):1149-1157) or humanized hepatocytes PXB mouse model (available at PhoenixBio, see also Kakuni et al 2014 Int. J. Mol. Sci. 15:58-74). Inhibition of secretion of HBsAg and/or HBeAg may be measured by ELISA, e.g. by using the CLIA ELISA Kit (Autobio Diagnostic) according to the manufacturers' instructions. Reduction of HBV mRNA and pgRNA may be measured by qPCR. Further methods for evaluating whether a test compound inhibits HBV infection are measuring secretion of HBV DNA by qPCR e.g. as described in WO 2015/173208 or using Northern Blot; in-situ hybridization, or immuno-fluorescence.

In one embodiment of the present invention the pharmaceutical combination as described herein provides an advantage over the corresponding mono-compound treatments. The advantage can for example be i) prolonged serum HBV-DNA reduction compared to mono-therapy; ii) delayed rebound in HBsAg compared to mono-therapy and/or iii) increased therapeutic window. The term “therapeutic window” or “pharmaceutical window” in relation to a drug is the range of drug dosages which can treat disease effectively without having toxic effects. In one embodiment of the invention, an increase in the therapeutic window can be achieved by the combination treatment as compared to mono-therapy.

The invention provides methods for treating or preventing HBV infection, comprising administering a therapeutically or prophylactically effective amount of a pharmaceutical combination of the present invention to a subject suffering from or susceptible to HBV infection. A further aspect of the invention relates to the use of the pharmaceutical combination of the present invention to inhibit development of or treat a chronic HBV infection.

One aspect of the present invention is a method of treating an individual infected with HBV, such as an individual with chronic HBV infection, comprising administering a pharmaceutically effective amount of an RNAi oligonucleotide targeting HBV and a pharmaceutically effective amount of an anti-PDL1 antisense oligonucleotide.

The invention also relates to an RNAi oligonucleotide targeting HBV for use as a medicament in a combination treatment. The invention also relates to an anti-PDL1 antisense oligonucleotide for use as a medicament in a combination treatment.

In particular, the RNAi oligonucleotide targeting HBV and the anti-PDL1 antisense oligonucleotide are for use in treatment of a hepatitis B virus infection.

One embodiment of the invention is the use of an RNAi oligonucleotide targeting HBV in the manufacture of a first medicament for treating a hepatitis B virus infection, such as a chronic HBV virus infection, wherein the first medicament is an RNAi oligonucleotide targeting HBV as described in the present application and wherein the first medicament is to be administered in combination with a second medicament, wherein the second medicament is an anti-PDL1 antisense oligonucleotide as described in the present application.

In one embodiment of the present invention the medical composition containing the RNAi oligonucleotide targeting HBV or the anti-PDL1 antisense oligonucleotide is to be administered as a subcutaneous dose. In a further embodiment of the present invention any TLR7 agonist is to be administered as an oral dose. The medical composition may be administered through different routes of administration and can follow different administration regiments.

The pharmaceutical combination according to the present invention is typically administered in an effective amount.

In one embodiment the RNAi oligonucleotide targeting HBV and the anti-PDL1 antisense oligonucleotide as described in the present application are administered subcutaneously with weekly or monthly dosing in between 24 and 72 weeks, such as between 36 and 60 weeks, such as 48 weeks. In the period with administration every other day there may be a 10 to 14 week, such as a 12 week period off treatment.

### Methods of Use

### I. Reducing HBsAg Expression

In some embodiments, methods are provided for delivering to a cell an effective amount any one of the pharmaceutical combinations of the present invention, particularly the RNAi oligonucleotide targeting HBV and anti-PDL1 antisense oligonucleotide as described herein, for purposes of reducing expression of HBsAg. Methods provided herein are useful in any appropriate cell type. In some embodiments, a cell is any cell that expresses HBV antigen (e.g., hepatocytes, macrophages, monocyte-derived cells, prostate cancer cells, cells of the brain, endocrine tissue, bone marrow, lymph nodes, lung, gall bladder, liver, duodenum, small intestine, pancreas, kidney, gastrointestinal tract, bladder, adipose and soft tissue and skin). In some embodiments, the cell is a primary cell that has been obtained from a subject and that may have undergone a limited number of passages, such that the cell substantially maintains its natural phenotypic properties. In some embodiments, a cell to which the oligonucleotide is delivered is ex vivo or in vitro (i.e., can be delivered to a cell in culture or to an organism in which the cell resides). In specific embodiments, methods are provided for delivering to a cell a pharmaceutical combination comprising effective amounts of the RNAi oligonucleotide targeting HBV and anti-PDL1 antisense oligonucleotide as described herein for purposes of reducing expression of HBsAg solely in hepatocytes.

In some embodiments, the oligonucleotide therapeutics in the pharmaceutical combinations of the invention can be introduced using appropriate nucleic acid delivery methods including injection of a solution containing the oligonucleotides, bombardment by particles covered by the oligonucleotides, exposing the cell or organism to a solution containing the oligonucleotides, or electroporation of cell membranes in the presence of the oligonucleotides. Other appropriate methods for delivering oligonucleotides to cells may be used, such as lipid-mediated carrier transport, chemical-mediated transport, and cationic liposome transfection such as calcium phosphate, and others.

The consequences of inhibition can be confirmed by an appropriate assay to evaluate one or more properties of a cell or subject, or by biochemical techniques that evaluate molecules indicative of HBV antigen expression (e.g., RNA, protein). In some embodiments, the extent to which an oligonucleotide of a pharmaceutical combination provided herein reduces levels of expression of HBV antigen is evaluated by comparing expression levels (e.g., mRNA or protein levels) of HBV antigen to an appropriate control (e.g., a level of HBV antigen expression in a cell or population of cells to which the pharmaceutical combination has not been delivered or to which a negative control has been delivered). In some embodiments, an appropriate control level of HBV antigen expression may be a predetermined level or value, such that a control level need not be measured every time. The predetermined level or value can take a variety of forms. In some embodiments, a predetermined level or value can be single cut-off value, such as a median or mean.

In some embodiments, administration of a pharmaceutical combination comprising an oligonucleotide as described herein, particularly an RNAi oligonucleotide described herein, results in a reduction in the level of HBV antigen (e.g., HBsAg) expression in a cell. In some embodiments, the reduction in levels of HBV antigen expression may be a reduction to 1% or lower, 5% or lower, 10% or lower, 15% or lower, 20% or lower, 25% or lower, 30% or lower, 35% or lower, 40% or lower, 45% or lower, 50% or lower, 55% or lower, 60% or lower, 70% or lower, 80% or lower, or 90% or lower compared with an appropriate control level of HBV antigen. The appropriate control level may be a level of HBV antigen expression in a cell or population of cells that has not been contacted with a pharmaceutical combination comprising an oligonucleotide, particularly an RNAi oligonucleotide, as described herein. In some embodiments, the effect of delivery of an oligonucleotide of a pharmaceutical combination of the present invention to a cell according to a method disclosed herein is assessed after a finite period of time. For example, levels of HBV antigen may be analyzed in a cell at least 8 hours, 12 hours, 18 hours, 24 hours; or at least one, two, three, four, five, six, seven, fourteen, twenty-one, twenty-eight, thirty-five, forty-two, forty-nine, fifty-six, sixty-three, seventy, seventy-seven, eighty-four, ninety-one, ninety-eight, 105, 112, 119, 126, 133, 140, or 147 days after introduction of the oligonucleotide into the cell.

In some embodiments, the reduction in the level of HBV antigen (e.g., HBsAg) expression persists for an extended period of time following administration. In some embodiments, a detectable reduction in HBsAg expression persists within a period of 7 to 70 days following administration of an oligonucleotide of the pharmaceutical combination of the present invention, particularly where the oligonucleotide is an antisense oligonucleotide. For example, in some embodiments, the detectable reduction persists within a period of 10 to 70, 10 to 60, 10 to 50, 10 to 40, 10 to 30, or 10 to 20 days following administration of the oligonucleotide. In some embodiments, the detectable reduction persists within a period of 20 to 70, 20 to 60, 20 to 50, 20 to 40, or 20 to 30 days following administration of the oligonucleotide of the pharmaceutical combination of the present invention, particularly where the oligonucleotide is an antisense oligonucleotide. In some embodiments, the detectable reduction persists within a period of 30 to 70, 30 to 60, 30 to 50, or 30 to 40 days following administration of the oligonucleotide of the pharmaceutical combination of the present invention, particularly where the oligonucleotide is an antisense oligonucleotide. In some embodiments, the detectable reduction persists within a period of 40 to 70, 40 to 60, 40 to 50, 50 to 70, 50 to 60, or 60 to 70 days following administration of the oligonucleotide of the pharmaceutical combination of the present invention, particularly where the oligonucleotide is an antisense oligonucleotide.

In some embodiments, a detectable reduction in HBsAg expression persists within a period of 2 to 21 weeks following administration of an oligonucleotide of a pharmaceutical combination of the present invention, particularly where the oligonucleotide is an antisense oligonucleotide. For example, in some embodiments, the detectable reduction persists within a period of 2 to 20, 4 to 20, 6 to 20, 8 to 20, 10 to 20, 12 to 20, 14 to 20, 16 to 20, or 18 to 20 weeks following administration of the oligonucleotide of the pharmaceutical combination of the present invention, particularly where the oligonucleotide is an antisense oligonucleotide. In some embodiments, the detectable reduction persists within a period of 2 to 16, 4 to 16, 6 to 16, 8 to 16, 10 to 16, 12 to 16, or 14 to 16 weeks following administration of the oligonucleotide of the pharmaceutical combination of the present invention, particularly where the oligonucleotide is an antisense oligonucleotide. In some embodiments, the detectable reduction persists within a period of 2 to 12, 4 to 12, 6 to 12, 8 to 12, or 10 to 12 weeks following administration of the oligonucleotide of the pharmaceutical combination of the present invention, particularly where the oligonucleotide is an antisense oligonucleotide. In some embodiments, the detectable reduction persists within a period of 2 to 10, 4 to 10, 6 to 10, or 8 to 10 weeks following administration of the oligonucleotide of the pharmaceutical combination of the present invention, particularly where the oligonucleotide is an antisense oligonucleotide.

In some embodiments, an oligonucleotide of the pharmaceutical combination of the present invention, particularly where the oligonucleotide is an antisense oligonucleotide, is delivered in the form of a transgene that is engineered to express the oligonucleotide (e.g., its sense and antisense strands) in a cell. In some embodiments, an oligonucleotide of the pharmaceutical combination of the present invention, particularly where the oligonucleotide is an antisense oligonucleotide is delivered using a transgene that is engineered to express any oligonucleotide disclosed herein. Transgenes may be delivered using viral vectors (e.g., adenovirus, retrovirus, vaccinia virus, poxvirus, adeno-associated virus or herpes simplex virus) or non-viral vectors (e.g., plasmids or synthetic mRNAs). In some embodiments, transgenes of the pharmaceutical combinations of the present invention can be injected directly to a subject.

### II. Treatment Methods

Aspects of the disclosure relate to methods for reducing HBsAg expression (e.g., reducing HBsAg expression) for the treatment of HBV infection in a subject. In some embodiments, the methods may comprise administering to a subject in need thereof a pharmaceutical combination comprising effective amounts of the RNAi oligonucleotide targeting HBV and anti-PDL1 antisense oligonucleotide as described herein. The present disclosure provides for both prophylactic and therapeutic methods of treating a subject at risk of (or susceptible to) HBV infection and/or a disease or disorder associated with HBV infection.

In certain aspects, the disclosure provides a method for preventing in a subject, a disease or disorder as described herein by administering to the subject a therapeutic agent (e.g., a pharmaceutical combination, an oligonucleotide or vector or transgene encoding same). In some embodiments, particularly where the oligonucleotide of the pharmaceutical combination is an RNAi oligonucleotide, the subject to be treated is a subject who will benefit therapeutically from a reduction in the amount of HBsAg protein, e.g., in the liver. Subjects at risk for the disease or disorder can be identified by, for example, one or a combination of diagnostic or prognostic assays known in the art (e.g., identification of liver cirrhosis and/or liver inflammation). Administration of a prophylactic agent can occur prior to the detection of or the manifestation of symptoms characteristic of the disease or disorder, such that the disease or disorder is prevented or, alternatively, delayed in its progression.

Methods described herein typically involve administering to a subject an effective amount of a pharmaceutical combination, that is, an amount capable of producing a desirable therapeutic result. A therapeutically acceptable amount may be an amount that is capable of treating a disease or disorder. The appropriate dosage for any one subject will depend on certain factors, including the subject's size, body surface area, age, the particular composition to be administered, the active ingredient(s) in the composition, time and route of administration, general health, and other drugs being administered concurrently.

In some embodiments, a subject is administered any one of the compositions of the pharmaceutical combinations disclosed herein either enterally (e.g., orally, by gastric feeding tube, by duodenal feeding tube, via gastrostomy or rectally), parenterally (e.g., subcutaneous injection, intravenous injection or infusion, intra-arterial injection or infusion, intraosseous infusion, intramuscular injection, intracerebral injection, intracerebroventricular injection, intrathecal), topically (e.g., epicutaneous, inhalational, via eye drops, or through a mucous membrane), or by direct injection into a target organ (e.g., the liver of a subject). Typically, oligonucleotides of the pharmaceutical combinations disclosed herein are administered intravenously or subcutaneously.

In some embodiments, the subject to be treated is a human or non-human primate or other mammalian subject. Other exemplary subjects include domesticated animals such as dogs and cats; livestock such as horses, cattle, pigs, sheep, goats, and chickens; and animals such as mice, rats, guinea pigs, and hamsters.

### EMBODIMENTS

The following embodiments of the present invention may be used in combination with any other embodiments described herein.

1. A pharmaceutical combination for treating HBV, comprising at least two HBV therapeutics selected from the group consisting of an RNAi oligonucleotide targeting HBV, an anti-PDL1 antisense oligonucleotide, a TLR7 agonist, interferon-alpha, an anti-HBV antibody, an antibody which antagonises PD1 signalling and a nucleotide analogue.

2. The pharmaceutical combination of embodiment 1, wherein the combination is any one of the combinations C1-C120, as listed in Tables 2 and 3.

3. The pharmaceutical combination of embodiment 1, wherein the combination comprises an RNAi oligonucleotide targeting HBV and an anti-PDL1 antisense oligonucleotide.

4. The pharmaceutical combination of embodiment 3, wherein the RNAi oligonucleotide is a siRNA oligonucleotide that targets HBsAg mRNA and reduces the expression of HBsAg mRNA.

5. The pharmaceutical combination of embodiment 3 or 4, wherein the RNAi oligonucleotide is an oligonucleotide comprising an antisense strand of 19 to 30 nucleotides in length, wherein the antisense strand comprises a region of complementarity to a sequence of HBsAg mRNA set forth as ACAANAAUCCUCACAAUA (SEQ ID NO: 1).

6. The pharmaceutical combination of any one of embodiments 3 to 5, wherein the RNAi oligonucleotide comprises a sense strand that has a region of complementarity to the sequence set forth as UUNUUGUGAGGAUUN (SEQ ID NO: 2).

7. The pharmaceutical combination of any one of embodiments 3 to 6, wherein the RNAi oligonucleotide comprises a sense strand comprising a sequence GACAANAAUCCUCACAAUAAGCAGCCGAAAGGCUGC (SEQ ID NO: 8) wherein one or more of the nucleotides of the -GAAA- sequence on the sense strand are conjugated to a GalNac moiety, preferably wherein the RNAi oligonucleotide further comprises an antisense strain comprising a sequence UUAUUGUGAGGAUUNUUGUCGG (SEQ ID NO: 4).

8. The pharmaceutical combination of any one of embodiments 3 to 7, wherein the RNAi oligonucleotide is an oligonucleotide comprising a sense strand forming a duplex region with an antisense strand, wherein:
the sense strand consists of a sequence GACAAAAAUCCUCACAAUAAGCAGCCGAAAGGCUGC (SEQ ID NO: 9) and comprising 2′-fluoro modified nucleotides at positions 3, 8-10, 12, 13, and 17, 2′-O-methyl modified nucleotides at positions 1, 2, 4-7, 11, 14-16, 18-26, and 31-36, and a phosphorothioate linkage between the nucleotides at positions 1 and 2, wherein each of the nucleotides of the -GAAA-sequence on the sense strand is conjugated to a monovalent GalNac moiety; and the antisense strand consists of a sequence UUAUUGUGAGGAUUUUUGUCGG (SEQ ID NO: 6) and comprising 2′-fluoro modified nucleotides at positions 2, 3, 5, 7, 8, 10, 12, 14, 16, and 19, 2′-O-methyl modified nucleotides at positions 1, 4, 6, 9, 11, 13, 15, 17, 18, and 20-22, and phosphorothioate linkages between nucleotides at positions 1 and 2, between nucleotides at positions 2 and 3, between nucleotides at positions 3 and 4, between nucleotides at positions 20 and 21, and between nucleotides at positions 21 and 22, wherein the 4′-carbon of the sugar of the 5′-nucleotide of the antisense strand comprises a methoxy phosphonate (MOP).

9. The pharmaceutical combination of any one of embodiments 3 to 8, wherein the RNAi oligonucleotide is an oligonucleotide comprising a sense strand forming a duplex region with an antisense strand, wherein:
the sense strand comprises a sequence GACAAAAAUCCUCACAAUAAGCAGCCGAAAGGCUGC (SEQ ID NO: 9) and comprising 2′-fluoro modified nucleotides at positions 3, 8-10, 12, 13, and 17, 2′-O-methyl modified nucleotides at positions 1, 2, 4-7, 11, 14-16, 18-26, and 31-36, and one phosphorothioate internucleotide linkage between the nucleotides at positions 1 and 2, wherein each of the nucleotides of the -GAAA- sequence on the sense strand is conjugated to a monovalent GalNac moiety, wherein the -GAAA- sequence comprises the structure:

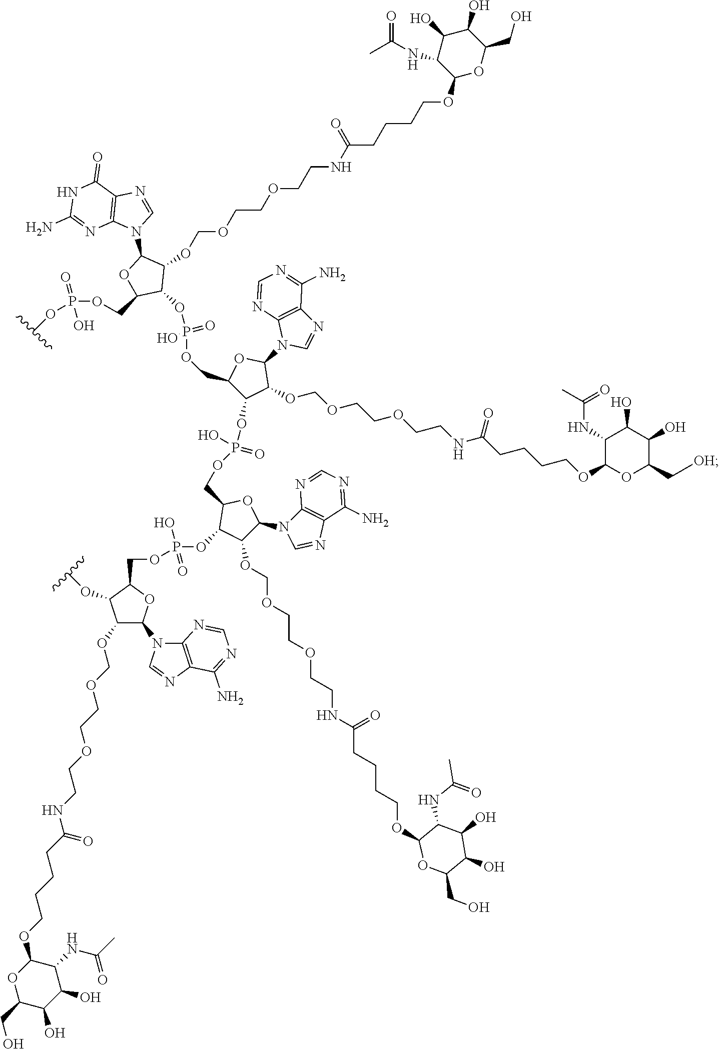 [SMILES: *O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(=O)[nH]c(N)nc32)O[C@@H]1COP(=O)(O)O*]

and
the antisense strand comprises a sequence as set forth in UUAUUGUGAGGAUUUUUGUCGG (SEQ ID NO: 6) and comprising 2′-fluoro modified nucleotides at positions 2, 3, 5, 7, 8, 10, 12, 14, 16, and 19, 2′-O-methyl modified nucleotides at positions 1, 4, 6, 9, 11, 13, 15, 17, 18, and 20-22, and five phosphorothioate internucleotide linkages between nucleotides 1 and 2, 2 and 3, 3 and 4, 20 and 21, and 21 and 22, wherein the 5′-nucleotide of the antisense strand has the following structure:

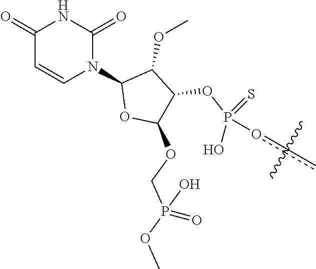

or a pharmaceutically acceptable salt thereof.

10. The pharmaceutical combination of any one of embodiments 3 to 9, wherein the anti-PDL1 antisense oligonucleotide is an antisense oligonucleotide that targets PDL1 and reduces the expression of PDL1.

11. The pharmaceutical combination of any one of embodiments 3 to 10, wherein the anti-PDL1 antisense oligonucleotide is an N-acetylgalactosamine (GalNAc)-conjugated locked nucleic acid (LNA) single-stranded oligonucleotide (SSO) that induces RNAseH-mediated degradation of PDL1 mRNA.

12. The pharmaceutical combination of any one of embodiments 3 to 11, wherein the anti-PDL1 antisense oligonucleotide comprises the sequence CCTATTTAACATCAGAC (SEQ ID NO: 11).

13. The pharmaceutical combination of any one of embodiments 3 to 12, wherein the anti-PDL1 antisense oligonucleotide has the formula GN2-C6ₒcₒaₒCCtatttaacatcAGAC, wherein C6 represents an amino alkyl group with 6 carbons, capital letters represent beta-D-oxy LNA nucleosides, lowercase letters represent DNA nucleosides, all LNA C are 5-methyl cytosine, subscript ₒ represents a phosphodiester nucleoside linkage, and unless otherwise indicated, all internucleoside linkages are phosphorothioate internucleoside linkages, and wherein GN2 represents the following trivalent GalNAc cluster:

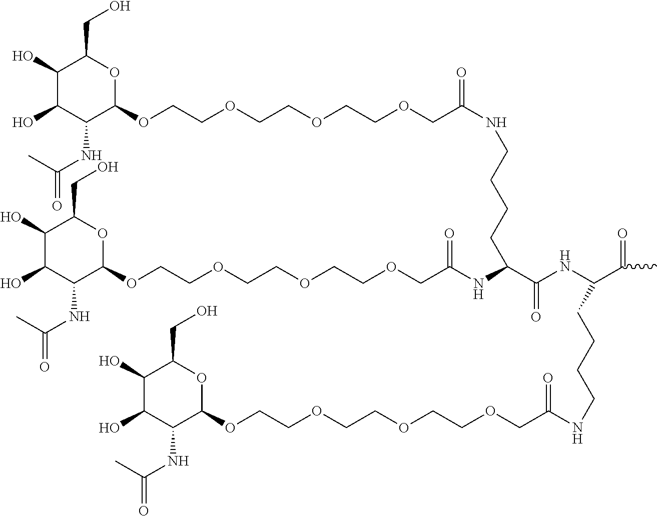

and further wherein the wavy line of the trivalent GalNAc cluster illustrates the site of conjugation of the trivalent GalNAc cluster to the C6 amino alkyl group;
or a pharmaceutically acceptable salt thereof.

14. The pharmaceutical combination of any one of embodiments 3 to 13, wherein the combination is capable of reducing serum HBsAg, HBeAg and/or HBV-DNA in a patient, relative to a vehicle control.

15. The pharmaceutical combination of any one of embodiments 3 to 14, wherein the combination is capable of reducing serum HBsAg, HBeAg and/or HBV-DNA in a patient, wherein the reduction is greater than a) the reduction provided by the same dose of the RNAi oligonucleotide targeting HBV when administered without an anti-PDL1 antisense oligonucleotide, and/or b) the reduction provided by the same dose of the anti-PDL1 antisense oligonucleotide when administered without an RNAi oligonucleotide targeting HBV.

16. The pharmaceutical combination of any one of embodiments 3 to 15, wherein the combination is capable of reducing serum HBsAg, HBeAg and/or HBV-DNA in a patient, wherein the reduction is greater than the sum of a) the reduction provided by the same dose of the RNAi oligonucleotide targeting HBV when administered without an anti-PDL1 antisense oligonucleotide, and b) the reduction provided by the same dose of the anti-PDL1 antisense oligonucleotide when administered without an RNAi oligonucleotide targeting HBV.

17. The pharmaceutical combination of any one of embodiments 3 to 16, wherein the RNAi oligonucleotide targeting HBV is present in an amount which will result in a dose of at least about 0.1 mg/kg to about 12 mg/kg, or a dose of at least about 0.5 mg/kg, or a dose of at least about 1 mg/kg, or a dose of at least about 1.5 mg/kg, or a dose of at least about 2 mg/kg, or a dose of at least about 3 mg/kg, or a dose of at least about 6 mg/kg, or a dose of at least about 9 mg/kg.

18. The pharmaceutical combination of any one of embodiments 3 to 17, wherein the RNAi oligonucleotide targeting HBV is present in an amount which will result in a dose of about 3 mg/kg to about 9 mg/kg, or a dose of about 3 mg/kg, or a dose of about 6 mg/kg, or a dose of about 9 mg/kg.

19. The pharmaceutical combination of any one of embodiments 3 to 18, wherein the RNAi oligonucleotide targeting HBV is present in an amount which will result in a dose of more than 3 mg/kg, or at least about 6 mg/kg, or at least about 9 mg/kg.

20. The pharmaceutical combination of any one of embodiments 3 to 19, wherein the anti-PDL1 antisense oligonucleotide is present in an amount which will result in a dose of at least about 0.1 mg/kg to about 35 mg/kg, or a dose of at least about 0.5 mg/kg, or a dose of at least about 1 mg/kg, or a dose of at least about 1.5 mg/kg, or a dose of at least about 2 mg/kg, or a dose of at least about 3 mg/kg, or a dose of at least about 6 mg/kg, or a dose of at least about 9 mg/kg.

21. The pharmaceutical combination of any one of embodiments 3 to 20, wherein the anti-PDL1 antisense oligonucleotide is present in an amount which will result in a dose of about 3 mg/kg, or about 6 mg/kg.

22. The pharmaceutical combination of any one of embodiments 3 to 21, wherein the anti-PDL1 antisense oligonucleotide is present in an amount which will result in a dose of more than 3 mg/kg, or at least about 6 mg/kg.

23. The pharmaceutical combination of any one of embodiments 3 to 20 or 22, wherein the anti-PDL1 antisense oligonucleotide is present in an amount which will result in a dose of about 7 mg/kg to about 35 mg/kg.

24. The pharmaceutical combination of any one of embodiments 3 to 23, wherein the pharmaceutical combination consists of or consists essentially of the RNAi oligonucleotide targeting HBV and the anti-PDL1 antisense oligonucleotide.

25. The pharmaceutical combination of any one of embodiments 3 to 23, further comprising a further, different HBV therapeutic.

26. The pharmaceutical combination of embodiment 25, wherein the further, different HBV therapeutic is a TLR7 agonist, interferon-alpha, an anti-HBV antibody, an antibody which inhibits PD1 signalling, or a nucleotide analogue.

27. The pharmaceutical combination of embodiment 26, wherein the further, different HBV therapeutic is a TLR7 agonist.

28. The pharmaceutical combination of any one of embodiments 3 to 27, wherein one or two or all of the HBV therapeutics are in the form of a pharmaceutically acceptable salt.

29. The pharmaceutical combination of any one of embodiments 3 to 28, wherein one or two or all of the HBV therapeutics are in the form of a prodrug.

30. The pharmaceutical combination of any one of embodiments 3 to 29, wherein one or two or all of the HBV therapeutics are each comprised in a composition with a pharmaceutically acceptable carrier, excipient, diluent or adjuvant.

31. A composition comprising the pharmaceutical combination according to any one of embodiments 3 to 30.

32. A kit of parts comprising the RNAi oligonucleotide targeting HBV according to any one of embodiments 3 to 29 and instructions for administration with an anti-PDL1 antisense oligonucleotide to treat a hepatitis B virus infection.

33. The kit of parts of embodiment 32, wherein the anti-PDL1 antisense oligonucleotide mentioned in the instructions is an anti-PDL1 antisense oligonucleotide according to any one of embodiments 3 to 30.

34. The kit of parts of embodiment 32 or 33, wherein the kit comprises an RNAi oligonucleotide targeting HBV according to embodiment 9 and an anti-PDL1 antisense oligonucleotide according to embodiment 13.

35. The kit of parts of any one of embodiments 32 to 34, wherein the RNAi oligonucleotide targeting HBV is formulated for subcutaneous injection and the anti-PDL1 antisense oligonucleotide is formulated for subcutaneous administration.

36. The kit of parts of any one of embodiments 32 to 35, wherein the instructions describe the treatment of a chronic hepatitis B virus infection.

37. The pharmaceutical combination, composition or kit of any one of embodiments 3 to 36, wherein the RNAi oligonucleotide targeting HBV and/or the anti-PDL1 antisense oligonucleotide are in the form of a transgene engineered to express the oligonucleotide in a cell.

38. Use of the pharmaceutical combination, composition or kit of any one of embodiments 3 to 37 for treating a hepatitis B virus infection.

39. The use of embodiment 38, wherein the initial or single dose of the RNAi oligonucleotide targeting HBV is administered prior to the administering of the initial or single dose of the anti-PDL1 antisense oligonucleotide.

40. The use of embodiment 38 or 39, wherein the single or initial dose of the anti-PDL1 antisense oligonucleotide is administered at least about a week, at least about two weeks, at least about three weeks, at least about four weeks, at least about five weeks, at least about six weeks, at least about seven weeks, at least about eight weeks or longer than eight weeks after the single or initial dose of the RNAi oligonucleotide targeting HBV.

41. The use of any one of embodiments 38 to 40, wherein the single or initial dose of the anti-PDL1 antisense oligonucleotide is administered at least about four weeks after the single or initial dose of the RNAi oligonucleotide targeting HBV.

42. The use of any one of embodiments 38 to 41, wherein the RNAi oligonucleotide targeting HBV is administered in weekly doses, and at least two doses are administered.

43. The use of any one of embodiments 38 to 42, wherein the anti-PDL1 antisense oligonucleotide is administered in weekly doses, and at least two doses are administered.

44. The use of any one of embodiments 38 to 43, wherein in the anti-PDL1 antisense oligonucleotide is administered in at least five doses.

45. The use of any one of embodiments 38 to 44, wherein the pharmaceutical combination is administered over the course of 48 weeks.

46. The use of any one of embodiments 38 to 45, wherein the RNAi oligonucleotide targeting HBV and the anti-PDL1 antisense oligonucleotide are administered in pharmaceutically effective amounts.

47. The use of any one of embodiments 38 to 46, wherein the RNAi oligonucleotide targeting HBV is administered in a dose of at least about 0.1 mg/kg, or a dose of at least about 0.5 mg/kg, or a dose of at least about 1 mg/kg, or a dose of at least about 1.5 mg/kg, or a dose of at least about 2 mg/kg, or a dose of at least about 3 mg/kg, or a dose of at least about 6 mg/kg, or a dose of at least about 9 mg/kg.

48. The use of any one of embodiments 38 to 47, wherein the RNAi oligonucleotide targeting HBV is administered in a dose of about 3 mg/kg to about 9 mg/kg, or a dose of about 3 mg/kg, or a dose of about 6 mg/kg, or a dose of about 9 mg/kg.

49. The use of any one of embodiments 38 to 48, wherein the RNAi oligonucleotide targeting HBV is administered in a dose of more than 3 mg/kg, or at least about 6 mg/kg, or at least about 9 mg/kg.

50. The use of any one of embodiments 38 to 49, wherein the anti-PDL1 antisense oligonucleotide is administered in a dose of at least about 0.1 mg/kg to about 35 mg/kg, or a dose of at least about 0.5 mg/kg, or a dose of at least about 1 mg/kg, or a dose of at least about 1.5 mg/kg, or a dose of at least about 2 mg/kg, or a dose of at least about 3 mg/kg, or a dose of at least about 6 mg/kg, or a dose of at least about 7 mg/kg, or a dose of about 7 mg/kg to about 35 mg/kg, preferably wherein the anti-PDL1 antisense oligonucleotide is administered in up to five doses of about 3 mg/kg, the doses being administered at least once every two weeks.

51. The use of any one of embodiments 38 to 50, wherein the anti-PDL1 antisense oligonucleotide is administered in a dose of about 3 mg/kg to about 6 mg/kg, or a dose of about 3 mg/kg, or a dose of about 6 mg/kg.

52. The use of any one of embodiments 38 to 51, wherein the anti-PDL1 antisense oligonucleotide is administered in a dose of more than 3 mg/kg, or at least about 6 mg/kg.

53. The use of any one of embodiments 38 to 52, wherein two or more, preferably at least five, doses of anti-PDL1 antisense oligonucleotide are administered, once weekly, with the initial dose of anti-PDL1 antisense oligonucleotide being administered at least about 7 days after the dose of the RNAi oligonucleotide targeting HBV; and the doses of the anti-PDL1 antisense oligonucleotide are at least about 3 mg/kg.

54. The use of any one of embodiments 38 to 53, wherein two or more, preferably at least five, doses of the anti-PDL1 antisense oligonucleotide are administered, once weekly, with the initial dose of the anti-PDL1 antisense oligonucleotide being administered at least about 7 days after the dose of the RNAi oligonucleotide targeting HBV; and the doses of the anti-PDL1 antisense oligonucleotide are more than 3 mg/kg, preferably at least about 6 mg/kg.

55. The use of any one of embodiments 38 to 54, wherein two or more, preferably at least five, doses of the anti-PDL1 antisense oligonucleotide are administered, once weekly, with the initial dose of the anti-PDL1 antisense oligonucleotide being administered at least about 7 days after the dose of the RNAi oligonucleotide targeting HBV; and the dose of the RNAi oligonucleotide targeting HBV is more than 3 mg/kg, preferably at least about 9 mg/kg.

56. The use of any one of embodiments 38 to 55, wherein two or more, preferably at least five, doses of the anti-PDL1 antisense oligonucleotide are administered, once weekly, with the initial dose of the anti-PDL1 antisense oligonucleotide being administered at least about 7 days after the dose of the RNAi oligonucleotide targeting HBV; and the dose of the RNAi oligonucleotide targeting HBV is more than 3 mg/kg, preferably at least about 9 mg/kg; and each dose of the anti-PDL1 antisense oligonucleotide is more than 3 mg/kg, preferably at least about 6 mg/kg.

57. The use of any one of embodiments 38 to 56, wherein the hepatitis B virus infection to be treated is a chronic hepatitis B virus infection.

58. The use of any one of embodiments 38 to 57, wherein the RNAi oligonucleotide targeting HBV is in a dosage form for subcutaneous administration, and the anti-PDL1 antisense oligonucleotide is in a dosage form for subcutaneous administration.

59. The use of any one of embodiments 38 to 58, wherein the pharmaceutical combination is administered in the absence of treatment with an RNAi oligonucleotide targeting a non-surface antigen encoding HBV mRNA transcript.

60. The use of any one of embodiments 38 to 59, wherein the subject is not administered an RNAi oligonucleotide that selectively targets HBxAg mRNA transcript.

61. The use of any one of embodiments 38 to 60, further comprising administering to the subject an effective amount of Entecavir.

62. The use of any one of embodiments 38 to 61, wherein the RNAi oligonucleotide targeting HBV and/or the anti-PDL1 antisense oligonucleotide are delivered in the form of a transgene that is engineered to express the oligonucleotide in a cell.

63. The pharmaceutical combination, composition or kit of any one of embodiments 3 to 37, for use in medicine.

64. The pharmaceutical combination, composition or kit of any one of embodiments 3 to 37, for use in treatment of a hepatitis B virus infection.

65. The pharmaceutical combination, composition or kit for use of embodiment 63 or 64, wherein the single or initial dose of the RNAi oligonucleotide targeting HBV is administered prior to the administering of the single or initial dose of the anti-PDL1 antisense oligonucleotide.

66. The pharmaceutical combination, composition or kit for use of any one of embodiments 63 to 65, wherein the single or initial dose of the anti-PDL1 antisense oligonucleotide is administered at least about a week, at least about two weeks, at least about three weeks, at least about four weeks, at least about five weeks, at least about six weeks, at least about seven weeks, at least about eight weeks or longer than eight weeks after the single or initial dose of the RNAi oligonucleotide targeting HBV.

67. The pharmaceutical combination, composition or kit for use of any one of embodiments 63 to 66, wherein the single or initial dose of the anti-PDL1 antisense oligonucleotide is administered at least about four weeks after the single or initial dose of the RNAi oligonucleotide targeting HBV.

68. The pharmaceutical combination, composition or kit for use of any one of embodiments 63 to 67, wherein the RNAi oligonucleotide targeting HBV is administered in weekly doses, and at least two doses are administered.

69. The pharmaceutical combination, composition or kit for use of any one of embodiments 63 to 68, wherein the anti-PDL1 antisense oligonucleotide is administered in weekly doses, and at least two doses are administered.

70. The pharmaceutical combination, composition or kit for use of any one of embodiments 63 to 69, wherein in the anti-PDL1 antisense oligonucleotide is administered in at least five doses.

71. The pharmaceutical combination, composition or kit for use of any one of embodiments 63 to 70, wherein the pharmaceutical combination is administered over the course of 48 weeks.

72. The pharmaceutical combination, composition or kit for use of any one of embodiments 63 to 71, wherein the RNAi oligonucleotide targeting HBV and the anti-PDL1 antisense oligonucleotide are administered in pharmaceutically effective amounts.

73. The pharmaceutical combination, composition or kit for use of any one of embodiments 63 to 72, wherein the RNAi oligonucleotide targeting HBV is administered in a dose of at least about 0.1 mg/kg, or a dose of at least about 0.5 mg/kg, or a dose of at least about 1 mg/kg, or a dose of at least about 1.5 mg/kg, or a dose of at least about 2 mg/kg, or a dose of at least about 3 mg/kg, or a dose of at least about 6 mg/kg, or a dose of at least about 9 mg/kg.

74. The pharmaceutical combination, composition or kit for use of any one of embodiments 63 to 73, wherein the RNAi oligonucleotide targeting HBV is administered in a dose of about 3 mg/kg to about 9 mg/kg, or a dose of about 3 mg/kg, or a dose of about 6 mg/kg, or a dose of about 9 mg/kg.

75. The pharmaceutical combination, composition or kit for use of any one of embodiments 63 to 74, wherein the RNAi oligonucleotide targeting HBV is administered in a dose of more than 3 mg/kg, or at least about 6 mg/kg, or at least about 9 mg/kg.

76. The pharmaceutical combination, composition or kit for use of any one of embodiments 63 to 75, wherein the anti-PDL1 antisense oligonucleotide is administered in a dose of at least about 0.1 mg/kg to about 35 mg/kg, or a dose of at least about 0.5 mg/kg, or a dose of at least about 1 mg/kg, or a dose of at least about 1.5 mg/kg, or a dose of at least about 2 mg/kg, or a dose of at least about 3 mg/kg, or a dose of at least about 6 mg/kg, or a dose of at least about 7 mg/kg, or a dose of about 7 mg/kg to about 35 mg/kg, preferably wherein the anti-PDL1 antisense oligonucleotide is administered in up to five doses of about 3 mg/kg, the doses being administered at least once every two weeks.

77. The pharmaceutical combination, composition or kit for use of any one of embodiments 63 to 76, wherein the anti-PDL1 antisense oligonucleotide is administered in a dose of about 3 mg/kg to about 6 mg/kg, or a dose of about 3 mg/kg, or a dose of about 6 mg/kg.

78. The pharmaceutical combination, composition or kit for use of any one of embodiments 63 to 77, wherein the anti-PDL1 antisense oligonucleotide is administered in a dose of more than 3 mg/kg, or at least about 6 mg/kg.

79. The pharmaceutical combination, composition or kit for use of any one of embodiments 63 to 78, wherein two or more, preferably at least five, doses of anti-PDL1 antisense oligonucleotide are administered, once weekly, with the initial dose of anti-PDL1 antisense oligonucleotide being administered at least about 7 days after the dose of the RNAi oligonucleotide targeting HBV; and the doses of the anti-PDL1 antisense oligonucleotide are at least about 3 mg/kg.

80. The pharmaceutical combination, composition or kit for use of any one of embodiments 63 to 79, wherein two or more, preferably at least five, doses of the anti-PDL1 antisense oligonucleotide are administered, once weekly, with the initial dose of the anti-PDL1 antisense oligonucleotide being administered at least about 7 days after the dose of the RNAi oligonucleotide targeting HBV; and the doses of the anti-PDL1 antisense oligonucleotide are more than 3 mg/kg, preferably at least about 6 mg/kg.

81. The pharmaceutical combination, composition or kit for use of any one of embodiments 63 to 80, wherein two or more, preferably at least five, doses of the anti-PDL1 antisense oligonucleotide are administered, once weekly, with the initial dose of the anti-PDL1 antisense oligonucleotide being administered at least about 7 days after the dose of the RNAi oligonucleotide targeting HBV; and the dose of the RNAi oligonucleotide targeting HBV is more than 3 mg/kg, preferably at least about 9 mg/kg.

82. The pharmaceutical combination, composition or kit for use of any one of embodiments 63 to 81, wherein two or more, preferably at least five, doses of the anti-PDL1 antisense oligonucleotide are administered, once weekly, with the initial dose of the anti-PDL1 antisense oligonucleotide being administered at least about 7 days after the dose of the RNAi oligonucleotide targeting HBV; and the dose of the RNAi oligonucleotide targeting HBV is more than 3 mg/kg, preferably at least about 9 mg/kg; and each dose of the anti-PDL1 antisense oligonucleotide is more than 3 mg/kg, preferably at least about 6 mg/kg.

83. The pharmaceutical combination, composition or kit for use of any one of embodiments 63 to 82, wherein the hepatitis B virus infection to be treated is a chronic hepatitis B virus infection.

84. The pharmaceutical combination, composition or kit for use of any one of embodiments 63 to 83, wherein the RNAi oligonucleotide targeting HBV is in a dosage form for subcutaneous administration, and the anti-PDL1 antisense oligonucleotide is in a dosage form for subcutaneous administration.

85. The pharmaceutical combination, composition or kit for use of any one of embodiments 63 to 84, wherein the pharmaceutical combination is administered in the absence of treatment with an RNAi oligonucleotide targeting a non-surface antigen encoding HBV mRNA transcript.

86. The pharmaceutical combination, composition or kit for use of any one of embodiments 63 to 85, wherein the subject is not administered an RNAi oligonucleotide that selectively targets HBxAg mRNA transcript.

87. The pharmaceutical combination, composition or kit for use of any one of embodiments 63 to 86, further comprising administering to the subject an effective amount of Entecavir.

88. The pharmaceutical combination, composition or kit for use of any one of embodiments 63 to 87, wherein the RNAi oligonucleotide targeting HBV and/or the anti-PDL1 antisense oligonucleotide are delivered in the form of a transgene that is engineered to express the oligonucleotide in a cell.

89. Use of the pharmaceutical combination, composition or kit of any one of embodiments 3 to 37 in the manufacture of a medicament.

90. Use of the pharmaceutical combination, composition or kit of any one of embodiments 3 to 37 in the manufacture of a medicament for treating a hepatitis B virus infection.

91. The use of embodiment 89 or 90, wherein the single or initial dose of the RNAi oligonucleotide targeting HBV is administered prior to the administering of the single or initial dose of the anti-PDL1 antisense oligonucleotide.

92. The use of any one of embodiments 89 to 91, wherein the single or initial dose of the anti-PDL1 antisense oligonucleotide is administered at least about a week, at least about two weeks, at least about three weeks, at least about four weeks, at least about five weeks, at least about six weeks, at least about seven weeks, at least about eight weeks or longer than eight weeks after the single or initial dose of the RNAi oligonucleotide targeting HBV.

93. The use of any one of embodiments 89 to 92, wherein the single or initial dose of the anti-PDL1 antisense oligonucleotide is administered at least about four weeks after the single or initial dose of the RNAi oligonucleotide targeting HBV.

94. The use of any one of embodiments 89 to 93, wherein the RNAi oligonucleotide targeting HBV is administered in weekly doses, and at least two doses are administered.

95. The use of any one of embodiments 89 to 94, wherein the anti-PDL1 antisense oligonucleotide is administered in weekly doses, and at least two doses are administered.

96. The use of any one of embodiments 89 to 95, wherein in the anti-PDL1 antisense oligonucleotide is administered in at least five doses.

97. The use of any one of embodiments 89 to 96, wherein the pharmaceutical combination is administered over the course of 48 weeks.

98. The use of any one of embodiments 89 to 97, wherein the RNAi oligonucleotide targeting HBV and the anti-PDL1 antisense oligonucleotide are administered in pharmaceutically effective amounts.

99. The use of any one of embodiments 89 to 98, wherein the RNAi oligonucleotide targeting HBV is administered in a dose of at least about 0.1 mg/kg, or a dose of at least about 0.5 mg/kg, or a dose of at least about 1 mg/kg, or a dose of at least about 1.5 mg/kg, or a dose of at least about 2 mg/kg, or a dose of at least about 3 mg/kg, or a dose of at least about 6 mg/kg, or a dose of at least about 9 mg/kg.

100. The use of any one of embodiments 89 to 99, wherein the RNAi oligonucleotide targeting HBV is administered in a dose of about 3 mg/kg to about 9 mg/kg, or a dose of about 3 mg/kg, or a dose of about 6 mg/kg, or a dose of about 9 mg/kg.

101. The use of any one of embodiments 89 to 100, wherein the RNAi oligonucleotide targeting HBV is administered in a dose of more than 3 mg/kg, or at least about 6 mg/kg, or at least about 9 mg/kg.

102. The use of any one of embodiments 89 to 101, wherein the anti-PDL1 antisense oligonucleotide is administered in a dose of at least about 0.1 mg/kg to about 35 mg/kg, or a dose of at least about 0.5 mg/kg, or a dose of at least about 1 mg/kg, or a dose of at least about 1.5 mg/kg, or a dose of at least about 2 mg/kg, or a dose of at least about 3 mg/kg, or a dose of at least about 6 mg/kg, or a dose of at least about 7 mg/kg, or a dose of about 7 mg/kg to about 35 mg/kg, preferably wherein the anti-PDL1 antisense oligonucleotide is administered in up to five doses of about 3 mg/kg, the doses being administered at least once every two weeks.

103. The use of any one of embodiments 89 to 102, wherein the anti-PDL1 antisense oligonucleotide is administered in a dose of about 3 mg/kg to about 6 mg/kg, or a dose of about 3 mg/kg, or a dose of about 6 mg/kg.

104. The use of any one of embodiments 89 to 103, wherein the anti-PDL1 antisense oligonucleotide is administered in a dose of more than 3 mg/kg, or at least about 6 mg/kg.

105. The use of any one of embodiments 89 to 104, wherein two or more, preferably at least five, doses of anti-PDL1 antisense oligonucleotide are administered, once weekly, with the initial dose of anti-PDL1 antisense oligonucleotide being administered at least about 7 days after the dose of the RNAi oligonucleotide targeting HBV; and the doses of the anti-PDL1 antisense oligonucleotide are at least about 3 mg/kg.

106. The use of any one of embodiments 89 to 105, wherein two or more, preferably at least five, doses of the anti-PDL1 antisense oligonucleotide are administered, once weekly, with the initial dose of the anti-PDL1 antisense oligonucleotide being administered at least about 7 days after the dose of the RNAi oligonucleotide targeting HBV; and the doses of the anti-PDL1 antisense oligonucleotide are more than 3 mg/kg, preferably at least about 6 mg/kg.

107. The use of any one of embodiments 89 to 106, wherein two or more, preferably at least five, doses of the anti-PDL1 antisense oligonucleotide are administered, once weekly, with the initial dose of the anti-PDL1 antisense oligonucleotide being administered at least about 7 days after the dose of the RNAi oligonucleotide targeting HBV; and the dose of the RNAi oligonucleotide targeting HBV is more than 3 mg/kg, preferably at least about 9 mg/kg.

108. The use of any one of embodiments 89 to 107, wherein two or more, preferably at least five, doses of the anti-PDL1 antisense oligonucleotide are administered, once weekly, with the initial dose of the anti-PDL1 antisense oligonucleotide being administered at least about 7 days after the dose of the RNAi oligonucleotide targeting HBV; and the dose of the RNAi oligonucleotide targeting HBV is more than 3 mg/kg, preferably at least about 9 mg/kg; and each dose of the anti-PDL1 antisense oligonucleotide is more than 3 mg/kg, preferably at least about 6 mg/kg.

109. The use of any one of embodiments 89 to 108, wherein the hepatitis B virus infection to be treated is a chronic hepatitis B virus infection.

110. The use of any one of embodiments 89 to 109, wherein the RNAi oligonucleotide targeting HBV is in a dosage form for subcutaneous administration, and the anti-PDL1 antisense oligonucleotide is in a dosage form for subcutaneous administration.

111. The use of any one of embodiments 89 to 110, wherein the pharmaceutical combination is administered in the absence of treatment with an RNAi oligonucleotide targeting a non-surface antigen encoding HBV mRNA transcript.

112. The use of any one of embodiments 89 to 111, wherein the subject is not administered an RNAi oligonucleotide that selectively targets HBxAg mRNA transcript.

113. The use of any one of embodiments 89 to 112, further comprising administering to the subject an effective amount of Entecavir.

114. The use of any one of embodiments 89 to 113, wherein the RNAi oligonucleotide targeting HBV and/or the anti-PDL1 antisense oligonucleotide are delivered in the form of a transgene that is engineered to express the oligonucleotide in a cell.

115. A method for treating a hepatitis B virus infection comprising administering a therapeutically effective amount of the pharmaceutical combination, composition or kit of any one of embodiments 3 to 37 to a subject infected with a hepatitis B virus infection.

116. The method of embodiment 115, wherein the single or initial dose of the RNAi oligonucleotide targeting HBV is administered prior to the administering of the single or initial dose of the anti-PDL1 antisense oligonucleotide.

117. The method of embodiment 115 or 116, wherein the single or initial dose of the anti-PDL1 antisense oligonucleotide is administered at least about a week, at least about two weeks, at least about three weeks, at least about four weeks, at least about five weeks, at least about six weeks, at least about seven weeks, at least about eight weeks or longer than eight weeks after the single or initial dose of the RNAi oligonucleotide targeting HBV.

118. The method of any one of embodiments 115 to 117, wherein the single or initial dose of the anti-PDL1 antisense oligonucleotide is administered at least about four weeks after the single or initial dose of the RNAi oligonucleotide targeting HBV.

119. The method of any one of embodiments 115 to 118, wherein the RNAi oligonucleotide targeting HBV is administered in weekly doses, and at least two doses are administered.

120. The method of any one of embodiments 115 to 119, wherein the anti-PDL1 antisense oligonucleotide is administered in weekly doses, and at least two doses are administered.

121. The method of any one of embodiments 115 to 120, wherein in the anti-PDL1 antisense oligonucleotide is administered in at least five doses.

122. The method of any one of embodiments 115 to 121, wherein the pharmaceutical combination is administered over the course of 48 weeks.

123. The method of any one of embodiments 115 to 122, wherein the RNAi oligonucleotide targeting HBV and the anti-PDL1 antisense oligonucleotide are administered in pharmaceutically effective amounts.

124. The method of any one of embodiments 115 to 123, wherein the RNAi oligonucleotide targeting HBV is administered in a dose of at least about 0.1 mg/kg, or a dose of at least about 0.5 mg/kg, or a dose of at least about 1 mg/kg, or a dose of at least about 1.5 mg/kg, or a dose of at least about 2 mg/kg, or a dose of at least about 3 mg/kg, or a dose of at least about 6 mg/kg, or a dose of at least about 9 mg/kg.

125. The method of any one of embodiments 115 to 124, wherein the RNAi oligonucleotide targeting HBV is administered in a dose of about 3 mg/kg to about 9 mg/kg, or a dose of about 3 mg/kg, or a dose of about 6 mg/kg, or a dose of about 9 mg/kg.

126. The method of any one of embodiments 115 to 125, wherein the RNAi oligonucleotide targeting HBV is administered in a dose of more than 3 mg/kg, or at least about 6 mg/kg, or at least about 9 mg/kg.

127. The method of any one of embodiments 115 to 126, wherein the anti-PDL1 antisense oligonucleotide is administered in a dose of at least about 0.1 mg/kg to about 35 mg/kg, or a dose of at least about 0.5 mg/kg, or a dose of at least about 1 mg/kg, or a dose of at least about 1.5 mg/kg, or a dose of at least about 2 mg/kg, or a dose of at least about 3 mg/kg, or a dose of at least about 6 mg/kg, or a dose of at least about 7 mg/kg, or a dose of about 7 mg/kg to about 35 mg/kg, preferably wherein the anti-PDL1 antisense oligonucleotide is administered in up to five doses of about 3 mg/kg, the doses being administered at least once every two weeks.

128. The method of any one of embodiments 115 to 127, wherein the anti-PDL1 antisense oligonucleotide is administered in a dose of about 3 mg/kg to about 6 mg/kg, or a dose of about 3 mg/kg, or a dose of about 6 mg/kg.

129. The method of any one of embodiments 115 to 128, wherein the anti-PDL1 antisense oligonucleotide is administered in a dose of more than 3 mg/kg, or at least about 6 mg/kg.

130. The method of any one of embodiments 115 to 129, wherein two or more, preferably at least five, doses of anti-PDL1 antisense oligonucleotide are administered, once weekly, with the initial dose of anti-PDL1 antisense oligonucleotide being administered at least about 7 days after the dose of the RNAi oligonucleotide targeting HBV; and the doses of the anti-PDL1 antisense oligonucleotide are at least about 3 mg/kg.

131. The method of any one of embodiments 115 to 130, wherein two or more, preferably at least five, doses of the anti-PDL1 antisense oligonucleotide are administered, once weekly, with the initial dose of the anti-PDL1 antisense oligonucleotide being administered at least about 7 days after the dose of the RNAi oligonucleotide targeting HBV; and the doses of the anti-PDL1 antisense oligonucleotide are more than 3 mg/kg, preferably at least about 6 mg/kg.

132. The method of any one of embodiments 115 to 131, wherein two or more, preferably at least five, doses of the anti-PDL1 antisense oligonucleotide are administered, once weekly, with the initial dose of the anti-PDL1 antisense oligonucleotide being administered at least about 7 days after the dose of the RNAi oligonucleotide targeting HBV; and the dose of the RNAi oligonucleotide targeting HBV is more than 3 mg/kg, preferably at least about 9 mg/kg.

133. The method of any one of embodiments 115 to 132, wherein two or more, preferably at least five, doses of the anti-PDL1 antisense oligonucleotide are administered, once weekly, with the initial dose of the anti-PDL1 antisense oligonucleotide being administered at least about 7 days after the dose of the RNAi oligonucleotide targeting HBV; and the dose of the RNAi oligonucleotide targeting HBV is more than 3 mg/kg, preferably at least about 9 mg/kg; and each dose of the anti-PDL1 antisense oligonucleotide is more than 3 mg/kg, preferably at least about 6 mg/kg.

134. The method of any one of embodiments 115 to 133, wherein the hepatitis B virus infection to be treated is a chronic hepatitis B virus infection.

135. The method of any one of embodiments 115 to 134, wherein the RNAi oligonucleotide targeting HBV is in a dosage form for subcutaneous administration, and the anti-PDL1 antisense oligonucleotide is in a dosage form for subcutaneous administration.

136. The method of any one of embodiments 115 to 135, wherein the pharmaceutical combination is administered in the absence of treatment with an RNAi oligonucleotide targeting a non-surface antigen encoding HBV mRNA transcript.

137. The method of any one of embodiments 115 to 136, wherein the subject is not administered an RNAi oligonucleotide that selectively targets HBxAg mRNA transcript.

138. The method of any one of embodiments 115 to 137, further comprising administering to the subject an effective amount of Entecavir.

139. The method of any one of embodiments 115 to 138, wherein the RNAi oligonucleotide targeting HBV and/or the anti-PDL1 antisense oligonucleotide are delivered in the form of a transgene that is engineered to express the oligonucleotide in a cell.

140. A method of reducing expression of hepatitis B virus surface antigen in a cell, the method comprising delivering to the cell the pharmaceutical combination or composition of any one of embodiments 3 to 37.

141. The method of embodiment 140, wherein the cell is a hepatocyte.

142. The method of embodiment 140 or 141, wherein the cell is in vivo.

143. The method of embodiment 140 or 141, wherein the cell is in vitro.

144. The method of any one of embodiments 140 to 143, wherein the therapeutic oligonucleotide is delivered in the form of a transgene that is engineered to express the oligonucleotide in the cell.

145. The pharmaceutical combination, composition, kit, use or method substantially as described herein and with reference to the accompanying drawings.

### Surrogate Equivalent Molecules

The present invention concerns pharmaceutical combinations comprising the RNAi oligonucleotide targeting HBV as defined above, and the anti-PDL1 antisense oligonucleotide as defined above. Most specifically, these pharmaceutical combinations comprise the HBV therapeutics defined as T1 and T2 above.

In the Examples section below, for practical purposes, surrogate equivalent molecules were used in place of T1 and T2 as defined above. In particular, therapeutics tailored for use in mice were used. However, it will be understood by the skilled person that the results disclosed in the Examples herein with these surrogate molecules will be substantially equivalent to the results that can be obtained with other RNAi oligonucleotides targeting HBV and anti-PDL1 antisense oligonucleotides as defined herein, e.g. T1 and T2.

For example, the particular surrogates used in the Examples herein were used because the Examples specifically relate to mice. However, the skilled person would equally be able to use, e.g., T1 and T2 to obtain equivalent results in human cells, tissues and subjects.

Now that the skilled person is in possession of the results of the Examples herein regarding pharmaceutical combinations which comprise a surrogate RNAi oligonucleotide targeting HBV and anti-PDL1 antisense oligonucleotide, the skilled person will be able to obtain equivalent results with other appropriate RNAi oligonucleotides targeting HBV and anti-PDL1 antisense oligonucleotides as defined herein, e.g. T1 and T2 as defined above.

In particular, the surrogate RNAi oligonucleotide targeting HBV that was used in the Examples herein, which is equivalent to T1 and is designated “sT1”, is as follows: A sense strand of formula: 5′ mG-S-mA-fC-mA-mA-mG-mA-fA-fU-fC-mC-fU-fC-mA-mC-mA-fA-mU-mA-mA-mG-mC-mA-mG-mC-mC-mG-[ademA-GalNAc]-[ademA-GalNAc]-[ademA-GalNAc]-mG-mG-mC-mU-mG-mC 3′ (SEQ ID NO: 40);
hybridised to an antisense strand of formula: 5′ [MePhosphonate-4O-mU]-S-fU-S-fA-S-mU-fU-mG-fU-fG-mA-fG-mG-fA-mU-fU-mC-fU-mU-mG-fU-mC-S-mG-S-mG 3′ (SEQ ID NO: 41);
wherein: mX indicates a 2′-O-methyl ribonucleotide; fX indicates a 2′-fluoro-deoxyribonucleotide; [ademA-GalNAc] indicates a 2′-O-GalNAc-modified adenosine; [MePhosphonate-40-mU] indicates a 4′-O-monomethylphosphonate-2′-O-methyl uridine, and, of the linkages included, “-” denotes phosphodiester and “—S-” denotes phosphorothioate.

In particular, the surrogate anti-PDL1 antisense oligonucleotide that was used in the Examples herein, which is equivalent to T2 and is designated “sT2”, is as follows:

(SEQ ID NO: 42)
5′ [GalNAc-C6]-c-a-A-S-[5meC]-S-G-S-g-S-t-S-a-S-
t-S-t-S-t-S-t-S-c-S-a-S-c-S-A-S-G-S-G 3′

wherein upper case nucleotides indicates LNA; lower case nucleotides indicates DNA; [5meC] indicates 5-Methylcytosine LNA; [GalNAc-C6] indicates trivalent GalNAc conjugate with a C6 alkyl linker, and, of the linkages included, “-” denotes phosphodiester and “—S—” denotes phosphorothioate.

Thus, where “sT1” and “sT2” are referred to in the Examples and Drawings herein, this is a reference to these specific, equivalent, surrogate versions of T1 and T2 respectively.

### Example 1

### Summary

The purpose of the study was to investigate pharmacologic efficacy of test compounds in the pharmaceutical combinations of the present invention, in Adeno-Associated Virus-Hepatitis B Virus (AAV-HBV) mouse model. Equivalent surrogates of the RNAi oligonucleotide targeting HBV defined above as T1, “sT1”, and the anti-PDL1 oligonucleotide defined above as T2, “sT2”, were tested as monotherapies and as part of a pharmaceutical combination.

Sixty-eight (68) AAV-HBV infected mice were selected and sorted into 10 groups based on serum HBsAg, HBeAg, HBV-DNA levels and body weight on Pre-dose Day 28.

Vehicle (Group 01) was dosed once weekly during Day 0-35. sT2 at 6 mg/kg (Groups 06, 09 and 10) were dosed once weekly during Day 7-35. sT2 at 3 mg/kg (Groups 04 and 07) and 6 mg/kg (Groups 05 and 08) were dosed once weekly during Day 21-49. sT1 at 3 mg/kg (Groups 02 and 07-09) and 9 mg/kg (Groups 03 and 10) were dosed once on Day 0. All the compounds were administered by subcutaneous injection at 5 mL/kg.

During Day 0-91, animal clinical signs were monitored twice daily and body weight were measured twice weekly. Serum levels of HBsAg, HBeAg and HBV-DNA were measured once weekly.

### Materials and Methods

This study was conducted according to the procedures outlined below. There was no untoward circumstance that occurred during the course of the study that would have altered the quality or integrity of the data.

This study was conducted in accordance with Clinical Research Organisation (CRO) Standard Operating Procedures (SOPs) and Good Research Practice (GRP).

Recombinant AAV-HBV solution was provided by Sponsor, and diluted appropriately.

Serum levels of Hepatitis B surface antigen (HBsAg) and hepatitis B e-antigen (HBeAg) were detected by ARCHITECT i2000 (Abbott Laboratories, Lake Bluff, IL, USA) and supporting reagents. Serum HBV-DNA was detected by AB17500 (Applied Biosystems, Foster City, CA, USA) and detection kit (Sansure Biotech Inc., Changsha, Hunan, China).

Eighty-eight (88) male C57BL/6 mice aged 4-5 weeks were used.

Mice were group housed in polycarbonate cages with corncob bedding under controlled temperature (21-25° C.), humidity (40-70%), and a 12-hour light/12-hour dark cycle (7:00 AM to 7:00 PM lights on). Mice were provided ad libitum access to normal diet (Rodent Diet #5C02, PMI Nutrition International, LLC, IN, USA) and sterile water.

All procedures in this study were in compliance with local animal welfare legislation, CRO global policies and procedures, and the Guide for the Care and Use of Laboratory Animals.

The mice were acclimatized in the animal facility for 3 days (Acclimation Day 0-2) after arrival. On Pre-dose Day 0, all the mice were injected through tail vein with 1×10¹¹ vector genome of AAV-HBV in 200 μL of Phosphate Buffer Saline (PBS).

Animal clinical signs were monitored twice daily during acclimation and pre-treatment phases. Body weight was measured on Acclimation Day 0 and Pre-dose Day 28. On Pre-dose Day 28 (28 days after AAV-HBV injection), blood samples were collected via submandibular vein and centrifuged to prepare serum samples (15 μL per mouse). The serum samples were measured for HBsAg, HBeAg and HBV-DNA.

Based on the serum levels of viral markers and body weight measured on Pre-dose Day 28, 68 mice were selected and randomized into 10 groups, with 6-8 mice per group (Table 4).

Vehicle (Group 01) was dosed once weekly during Day 0-35. sT2 at 6 mg/kg (Groups 06, 09 and 10) were dosed once weekly during Day 7-35. sT2 at 3 mg/kg (Groups 04 and 07) and 6 mg/kg (Groups 05 and 08) were dosed once weekly during Day 21-49. sT6 at 3 mg/kg (Groups 02 and 07-09) and 9 mg/kg (Groups 03 and 10) were dosed once on Day 0. All the compounds were administered by subcutaneous injection at 5 mU/kg.

Animal clinical signs were monitored twice daily during Day 0-91. The mice in all groups were bled for serum preparation (15+15 μL per mouse) on Days 0, 7, 14, 21, 28, 35, 42, 49 and 56. 15 μL of plasma samples was used for quantitative detection of HBsAg, HBeAg and HBV DNA.

TABLE 4
Group design.

| | | Dose | Dose Vol | | Dose | | |
|---|---|---|---|---|---|---|---|
| | Test | (mg/ | (mL/kg/ | Dose | Fre- | Dose | No. |
| Grp | Article | kg) | dose) | Route | quency | Schedule | An |
| 01 | Vehicle | 0 | 5 | SC | QW | Day 0-35 | 6 |
| 02 | sT1 | 3 | 5 | SC | Once | Day 0 | 6 |
| 03 | sT1 | 9 | 5 | SC | Once | Day 0 | 6 |
| 04 | sT2 | 3 | 5 | SC | QW | Day 21-49 | 6 |
| 05 | sT2 | 6 | 5 | SC | QW | Day 21-49 | 6 |
| 06 | sT2 | 6 | 5 | SC | QW | Day 7-35 | 6 |
| 07 | sT1 + sT2 | 3 + 3 | 5 + 5 | SC | Once + | Day 0 + | 8 |
| | | | | | QW | Day 21-49 | |
| 08 | sT1 + sT2 | 3 + 6 | 5 + 5 | SC | Once + | Day 0 + | 8 |
| | | | | | QW | Day 21-49 | |
| 09 | sT1 + sT2 | 3 + 6 | 5 + 5 | SC | Once + | Day 0 + | 8 |
| | | | | | QW | Day 7-35 | |
| 10 | sT1 + sT2 | 9 + 6 | 5 + 5 | SC | Once + | Day 0 + | 8 |
| | | | | | QW | Day 7-35 | |

Abbreviations: Grp = Group; Vol = Volume; No. An = Number of animals; QW = Once weekly; SC = Subcutaneous (injection).

### Results

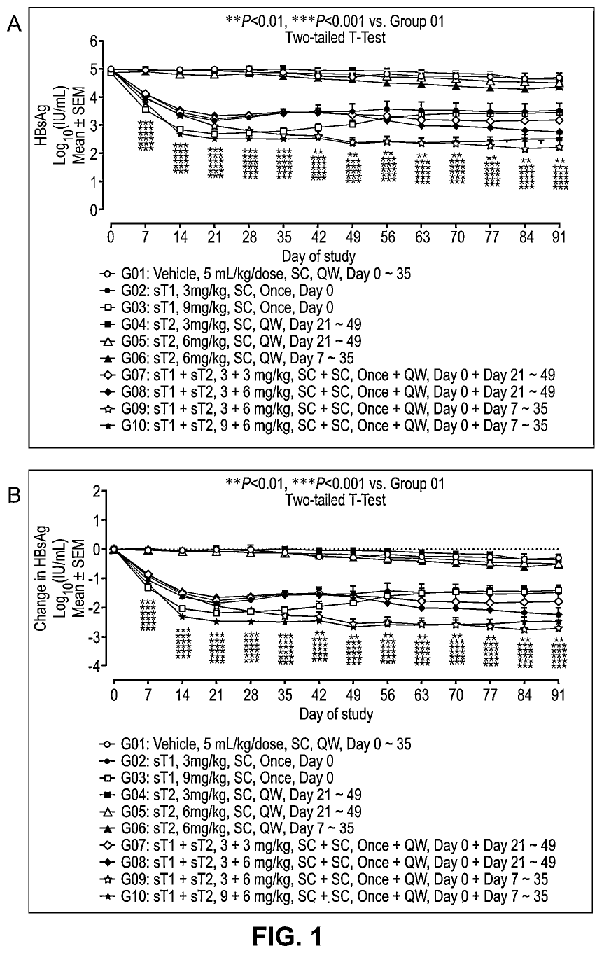

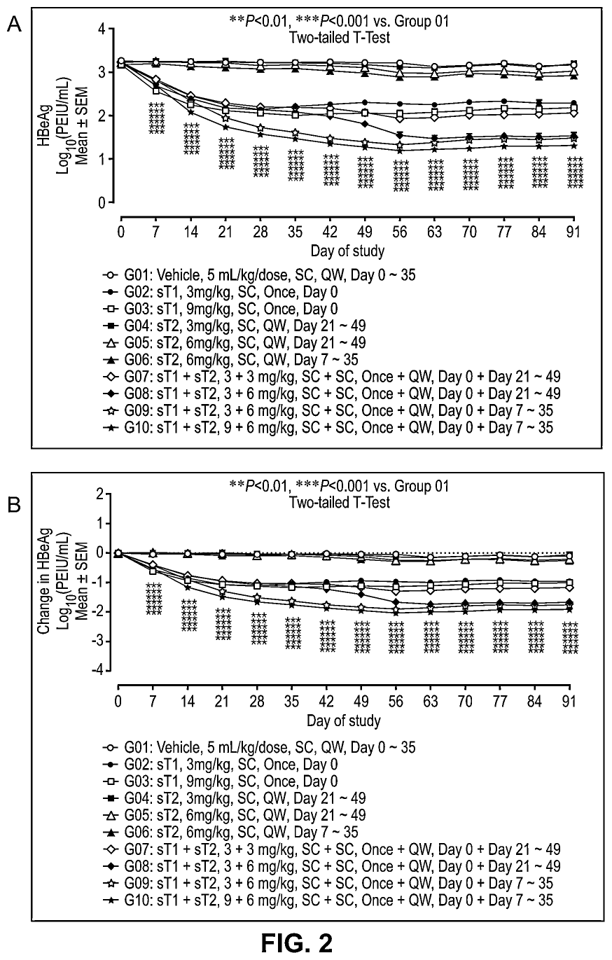

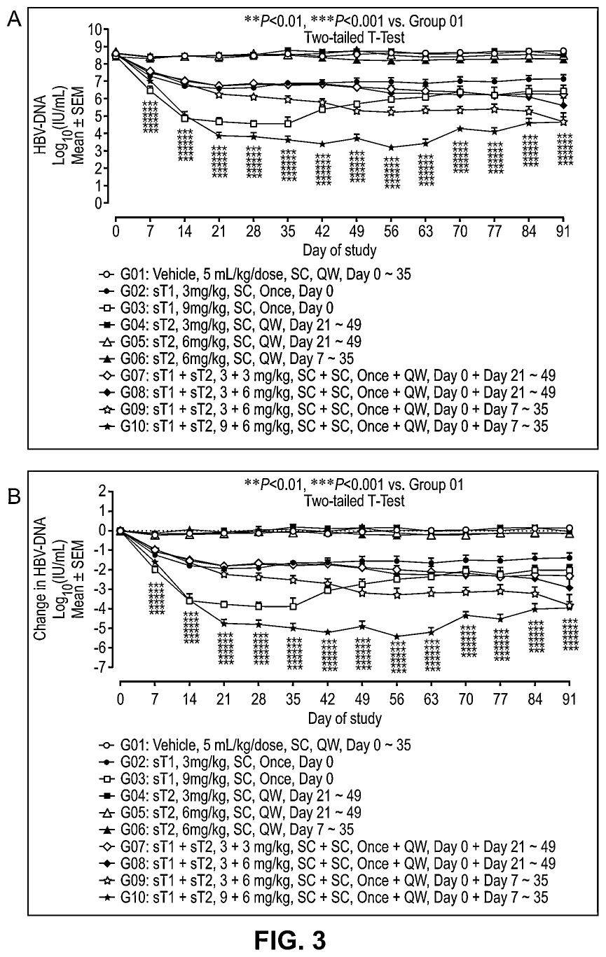

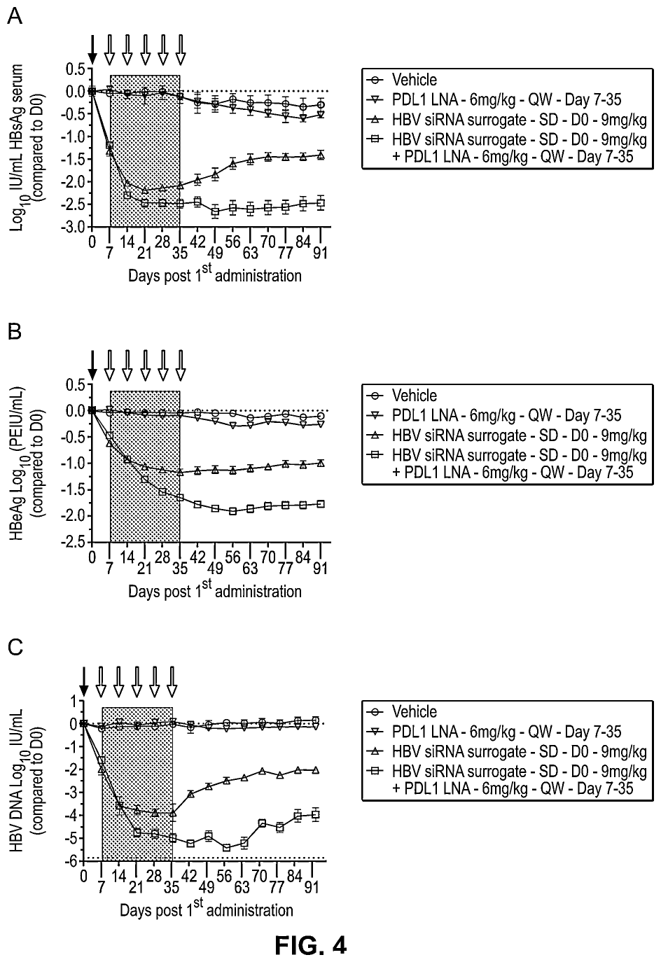

As shown in FIGS. 1-4, the serum levels of HBsAg, HBeAg and HBV-DNA in the vehicle control group maintained stable during Day 0-91. Compared to vehicle control group, treatment of a siRNA oligonucleotide targeting HBV (sT1) combined with an anti-PDL1 antisense oligonucleotide (sT2) continuously and significantly decreased serum levels of HBsAg, HBeAg and HBV-DNA during Day 7-91.

### Discussion

These results show the efficacy of pharmaceutical combinations comprising an RNAi oligonucleotide targeting HBV, such as T1 or sT1, and an anti-PDL1 antisense oligonucleotide, such as T2 or sT2, in vivo.

The pharmaceutical combination comprising both an RNAi oligonucleotide targeting HBV and an anti-PDL1 antisense oligonucleotide (G07-G10) achieved continuously and significantly decreased levels of the HBV serum markers HBsAg, HBeAg and HBV-DNA, indicating a strong inhibitory effect on HBV. Surprisingly, the effect obtained by this combination was both highly advantageous, eliciting a greater response than either individual therapeutic. The effect was also surprisingly synergistic, with a greater reduction in HBsAg, HBeAg and HBV-DNA serum levels than the sum of both the reduction obtained by an RNAi oligonucleotide targeting HBV alone (G02 and G03) and the reduction obtained by an anti-PDL1 antisense oligonucleotide alone (G04-G06).

Prior to these in vivo tests, the advantageous and synergistic effects on HBV of the pharmaceutical combination in G07-G10, which comprises both an RNAi oligonucleotide targeting HBV and an anti-PDL1 antisense oligonucleotide, could not have been foreseen. Without wishing to be bound by theory, it is considered that the therapeutic class of an RNAi oligonucleotide, particularly siRNA, for targeting HBV, when combined with the therapeutic class of an antisense oligonucleotide for targeting PDL1, contributes to the unexpectedly advantageous results. Advantageous forms and modifications of these therapeutics which are described herein, such as the general RNAi oligonucleotide sequence, GalNAc conjugation and the use of LNA, may also further contribute to the unexpectedly advantageous results. Now that these effects have been proven for the inventive pharmaceutical combination in vivo in mice (when utilising the surrogate therapeutics sT1 and sT2), it is to be understood that equivalent results can be obtained for the inventive pharmaceutical combination when using equivalent therapeutics, such as T1 and T2, in vivo in other cells/tissues/subjects, such as that of a human.

### Example 2

### Summary

The purpose of the study was to investigate pharmacologic efficacy of test compounds in the pharmaceutical combinations of the present invention, in Adeno-Associated Virus-Hepatitis B Virus (AAV-HBV) mouse model. Equivalent surrogates of the RNAi oligonucleotide targeting HBV defined above as T1, “sT1”, and the TLR7 agonist defined above as T3, “sT3”, were tested as monotherapies and as part of a pharmaceutical combination. AAV-HBV infected mice were selected and sorted into groups of 6 animals based on serum HBsAg, HBeAg, HBV-DNA levels and body weight on Pre-dose Day 28. Vehicle (Group 01) was dosed once at Day 0 with a saline control and once weekly from Day 21-56 with a buffer control. sT3 at 100 mg/kg was dosed weekly at Day 21-56 (Group 3 and 4). sT1 at 3 mg/kg was dosed once on Day 0 by subcutaneous injection at 5 mL/kg. sT3 was administered by orally at 10 mL/kg. During Day 0-140, animal clinical signs were monitored twice daily and body weight were measured twice weekly. Serum levels of HBsAg, HBeAg and HBV-DNA were measured once weekly.

### Materials and Methods

This study was conducted according to the procedures outlined below. There was no untoward circumstance that occurred during the course of the study that would have altered the quality or integrity of the data. This study was conducted in accordance with Clinical Research Organisation (CRO) Standard Operating Procedures (SOPs) and Good Research Practice (GRP). Recombinant AAV-HBV solution was provided by Sponsor, and diluted appropriately. Serum levels of Hepatitis B surface antigen (HBsAg) and hepatitis B e-antigen (HBeAg) were detected by ARCHITECT i2000 (Abbott Laboratories, Lake Bluff, IL, USA) and supporting reagents. Serum HBV-DNA was detected by AB17500 (Applied Biosystems, Foster City, CA, USA) and detection kit (Sansure Biotech Inc., Changsha, Hunan, China). C57BL/6 male mice aged 4-5 weeks were used. Mice were group housed in polycarbonate cages with corncob bedding under controlled temperature (21-25° C.), humidity (40-70%), and a 12-hour light/12-hour dark cycle (7:00 AM 25 to 7:00 PM lights on). Mice were provided ad libitum access to normal diet Rodent Diet #5C02 (PMI Nutrition International, LLC, IN, USA) and sterile water. All procedures in this study were in compliance with local animal welfare legislation, CRO global policies and procedures, and the Guide for the Care and Use of Laboratory Animals. The mice were acclimatized in the animal facility for 6 days (Acclimation Day 0-5) after arrival. On Pre-dose Day 0, all the mice were injected through tail vein with 1×10¹¹ vector genome of AAV-HBV in 200 μL of Phosphate Buffer Saline (PBS). Animal clinical signs were monitored twice daily during acclimation and pre-treatment phases. Body weight was measured on Acclimation Day 0 and Pre-dose Day 28. On Pre-dose Day 28 (28 days after AAV-HBV injection), blood samples were collected via submandibular vein and centrifuged to prepare serum samples (10 μL per mouse). The serum samples were measured for HBsAg, HBeAg and HBV-DNA. Based on the serum levels of viral markers and body weight measured on Pre-dose Day 28, mice were selected and randomized into groups, with 6 per group (Table 4). Vehicle (Group 01) was dosed once at Day 0 with a saline control and weekly at Day 21, 28, 35, 42, 56 with a buffer control. sT3 at 100 mg/kg was dosed weekly at Day 21, 28, 35, 42, 56 in monotherapy (Group 3) or combination therapy with sT1 at 3 mg/kg dosed once on Day 0 (Groups 04). sT1 was administered by subcutaneous injection at 5 mL/kg, sT3 was administered orally at 10 mL/kg. Animal clinical signs were monitored twice daily during Day 0-140 and body weight was measured twice weekly. Serum levels of HBsAg, HBeAg and HBV DNA were measured weekly from Day 0 to 140.

TABLE 4
Group design.

| | Test | Dose Level | Dose Vol | Dose | Dose | Dose | No. |
|---|---|---|---|---|---|---|---|
| Grp | Article | (mg/kg/dose) | (mL/kg/dose) | Route | Frequency | Schedule | An |
| 01 | Vehicle 1 + | 0 + 0 | 5 + 10 | SC + | Once + QW | Day 0 + | 6 |
| | Vehicle 2 | | | PO | | Day 21-56 | |
| 02 | sT1 | 3 | 5 | SC | Once | Day 0 | 6 |
| 03 | sT3 | 100 | 10 | PO | QW | Day 21-56 | 6 |
| 04 | sT1 + sT3 | 3 + 100 | 5 + 10 | SC + | Once + | Day 0 + | 6 |
| | | | | PO | QW | Day 21-56 | |

### Results & Discussion

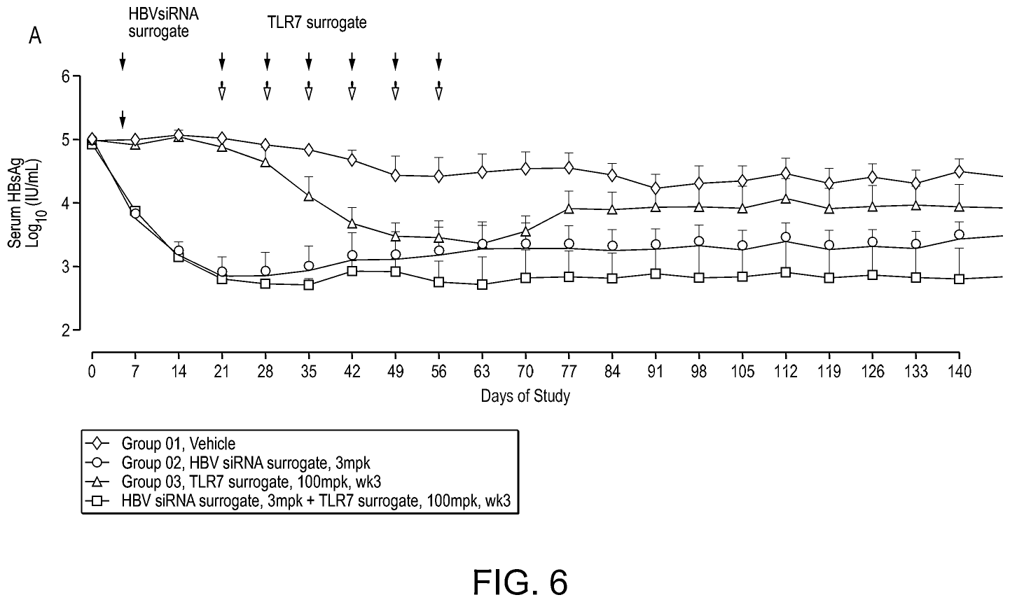

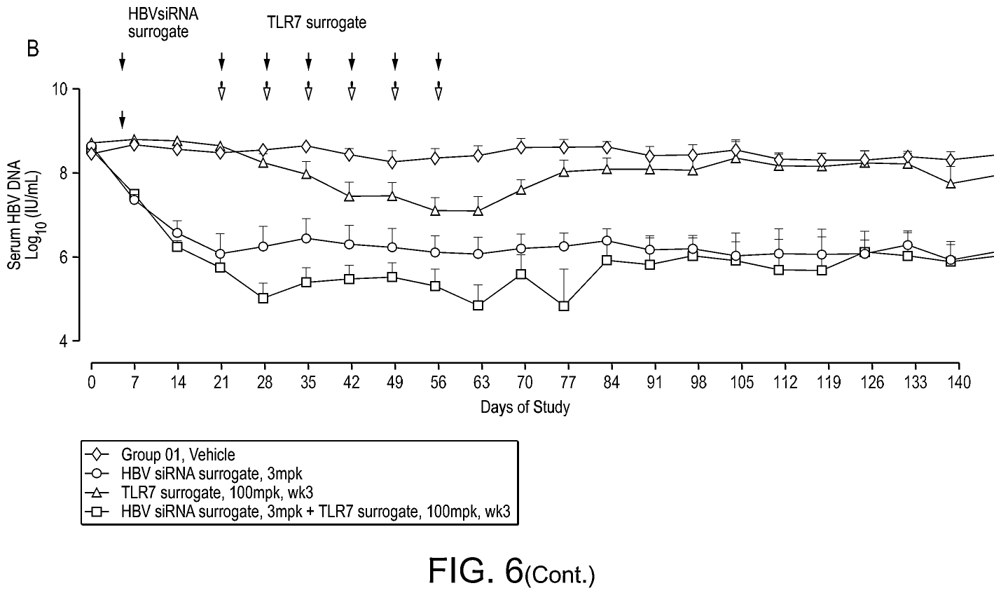

As shown in FIGS. 6A and 6B, the serum levels of HBsAg and HBV-DNA in the vehicle control group maintained at a relatively stable level during Day 0-140. Compared to the monotherapy groups of siRNA oligonucleotide targeting HBV (sT1, Group 02) and TLR7 agonist (sT3, Group 03) the combination of a siRNA oligonucleotide targeting HBV with a TLR7 agonist (sT1+sT3, Group 04) shows an additional reduction of serum levels of HBsAg and HBV-DNA.

These results show the efficacy of pharmaceutical combinations comprising an RNAi oligonucleotide targeting HBV, such as T1, and a TLR7 agonist, such as T3, in vivo. The pharmaceutical combination comprising both an RNAi oligonucleotide targeting HBV and a TLR7 agonist (Group 04) shows an additional decrease in levels of the HBV serum markers HBsAg and HBV-DNA, indicating an inhibitory effect on HBV. Now that these effects have been proven for the inventive pharmaceutical combination in vivo in mice (when utilizing the surrogate therapeutics sT1 and sT3), it is to be understood that equivalent results can be obtained for the inventive pharmaceutical combination when using equivalent therapeutics, such as T1 and T3, in vivo in other cells/tissues/subjects, such as that of a human.

### Example 3

### Summary

The purpose of the study was to investigate the pharmacologic efficacy of test compounds in the pharmaceutical combinations of the present invention, in Adeno-Associated Virus-Hepatitis B Virus (AAV-HBV) SCID mouse model. An equivalent surrogate of the RNAi oligonucleotide targeting HBV defined above as T1, “sT1”, and the anti-HBV antibody defined above as T5, “T5”, were tested as monotherapies and as part of a pharmaceutical combination. In particular, in this study T5 was an antibody comprising a VH domain comprising a) CDR-H1 comprising SEQ ID NO:12, b) CDR-H2 comprising SEQ ID NO:13, and c) CDR-H3 comprising SEQ ID NO:14, and a VL domain comprising d) CDR-L1 comprising SEQ ID NO:15, e) CDR-L2 comprising SEQ ID NO:16, and f) CDR-L3 comprising SEQ ID NO:17. More particularly, in this study T5 was an antibody comprising a VH domain comprising SEQ ID NO: 39, and a VL domain comprising SEQ ID NO: 37. More particularly, in this study T5 was an antibody comprising a heavy chain comprising SEQ ID NO: 38, and a light chain comprising SEQ ID NO: 36.

AAV-HBV infected SCID mice were selected and sorted into groups of 6 animals based on serum HBsAg, HBeAg, HBV-DNA levels and body weight on Pre-dose Day 14. Vehicle (Group 01) was dosed once at Day 0 with a saline control and at Day 21, 25, 29, 33 with a buffer control. T5 at 20 mg/kg was dosed at Day 21, 25, 29, 33 (Group 3 and 6). sT1 at 9 mg/kg was dosed once on Day 0 by subcutaneous injection at 5 mL/kg. T5 was administered by intravenous injection at 10 mL/kg. During Day 0-77, animal clinical signs were monitored twice daily and body weight were measured twice weekly. Serum levels of HBsAg, HBeAg and HBV-DNA were measured once weekly.

### Materials and Methods

This study was conducted according to the procedures outlined below. There was no untoward circumstance that occurred during the course of the study that would have altered the quality or integrity of the data. This study was conducted in accordance with Clinical Research Organisation (CRO) Standard Operating Procedures (SOPs) and Good Research Practice (GRP). Recombinant AAV-HBV solution was provided by Sponsor, and diluted appropriately. Serum levels of Hepatitis B surface antigen (HBsAg) and hepatitis B e-antigen (HBeAg) were detected by ARCHITECT i2000 (Abbott Laboratories, Lake Bluff, IL, USA) and 20 supporting reagents. Serum HBV-DNA was detected by AB17500 (Applied Biosystems, Foster City, CA, USA) and detection kit (Sansure Biotech Inc., Changsha, Hunan, China). CB17 SCID male mice aged 4-5 weeks were used. Mice were group housed in polycarbonate cages with corncob bedding under controlled temperature (21-25° C.), humidity (40-70%), and a 12-hour light/12-hour dark cycle (7:00 AM 25 to 7:00 PM lights on). Mice were provided ad libitum access to normal diet (Rodent Diet #5CJL, PMI Nutrition International, LLC, IN, USA) and sterile water. All procedures in this study were in compliance with local animal welfare legislation, CRO global policies and procedures, and the Guide for the Care and Use of Laboratory Animals. The mice were acclimatized in the animal facility for 6 days (Acclimation Day 0-5) after arrival. On Pre-dose Day 0, all the mice were injected through tail vein with 1×10¹¹ vector genome of AAV-HBV in 200 μL of Phosphate Buffer Saline (PBS). Animal clinical signs were monitored twice daily during acclimation and pre-treatment phases. Body weight was measured on Acclimation Day 0 and Pre-dose Day 14. On Pre-dose Day 14 (14 days after AAV-HBV injection), blood samples were collected via submandibular vein and centrifuged to prepare serum samples (10 μL per mouse). The serum samples were measured for HBsAg, HBeAg and HBV-DNA. Based on the serum levels of viral markers and body weight measured on Pre-dose Day 14, mice were selected and randomized into groups, with 6 per group (Table 5). Vehicle (Group 01) was dosed once at Day 0 with a saline control and at Day 21, 25, 29, 33 with a buffer control. T5 at 20 mg/kg was dosed Day 21, 25, 29, 33 in monotherapy (Group 3) or combination therapy with sT1 at 9 mg/kg dosed once on Day 0 (Group 06). sT1 was administered by subcutaneous injection at 5 mL/kg, T5 and the IgG control was administered by intravenous injection at 10 mL/kg. Animal clinical signs were monitored twice daily during Day 0-77 and body weight was measured twice weekly. Serum levels of HBsAg, HBeAg and HBV DNA were measured on Days 0, 7, 14, 21, 25, 28, 33, 35, 42, 49, 56, 63, 70 and 77.

TABLE 5
Group design.

| | | Dose Level | Dose Vol | Dose | Dose | Dose | No. |
|---|---|---|---|---|---|---|---|
| Grp | Test Article | (mg/kg/dose) | (mL/kg/dose) | Route | Frequency | Schedule | An |
| 01 | Vehicle 1 + | 0 + 0 | 5 + 10 | SC + | Once + | Day 0 + | 6 |
| | Vehicle 2 | | | IV | Q4D | Day 21-33 | |
| 02 | sT1 | 9 | 5 | SC | Once | Day 0 | 6 |
| 03 | T5 | 20 | 10 | IV | Q4D | Day 21-33 | 6 |
| 04 | IgG control | 20 | 10 | IV | Q4D | Day 21-33 | 6 |
| 05 | sT1 + IgG | 9 + 20 | 5 + 10 | SC + | Once + | Day 0 + | 6 |
| | control | | | IV | Q4D | Day 21-33 | |
| 06 | sT1 + T5 | 9 + 20 | 5 + 10 | SC + | Once + | Day 0 + | 6 |
| | | | | IV | Q4D | Day 21-33 | |

### Results

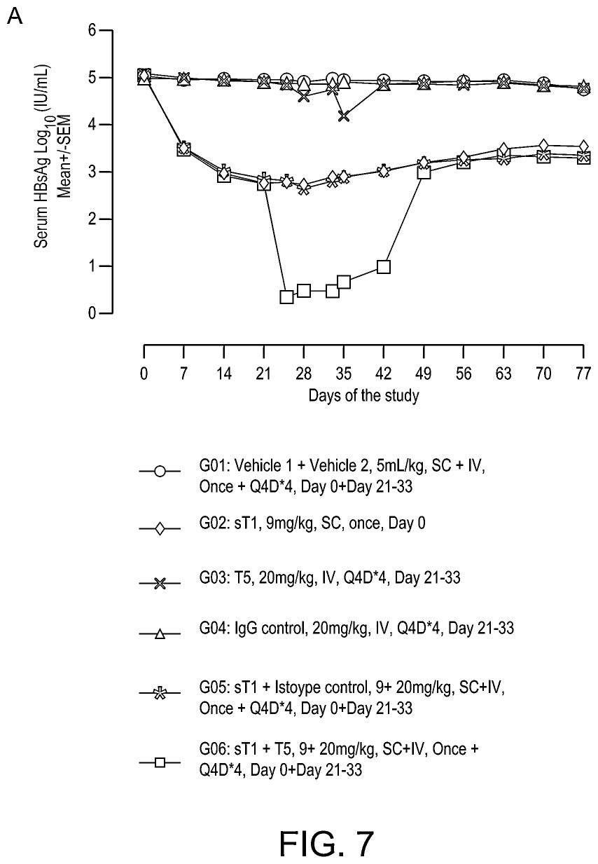

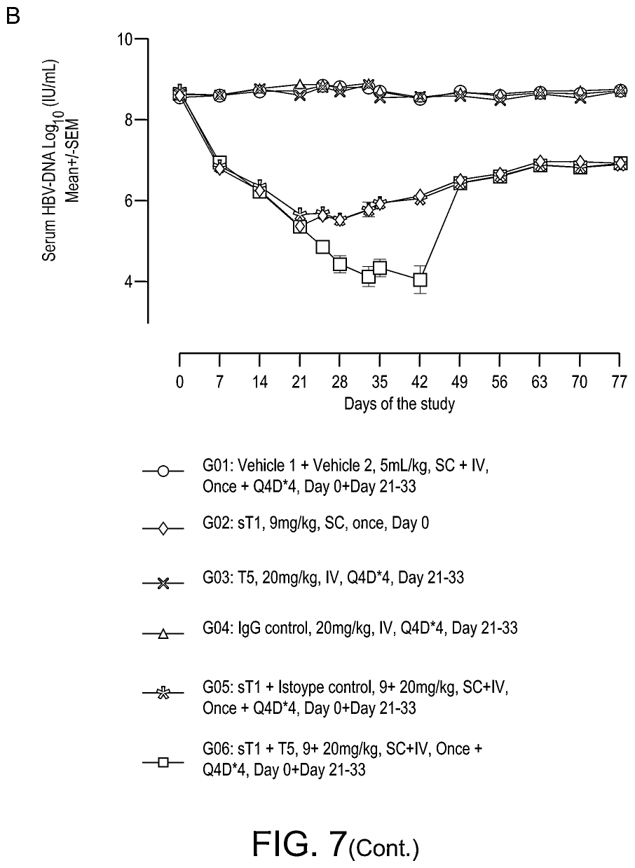

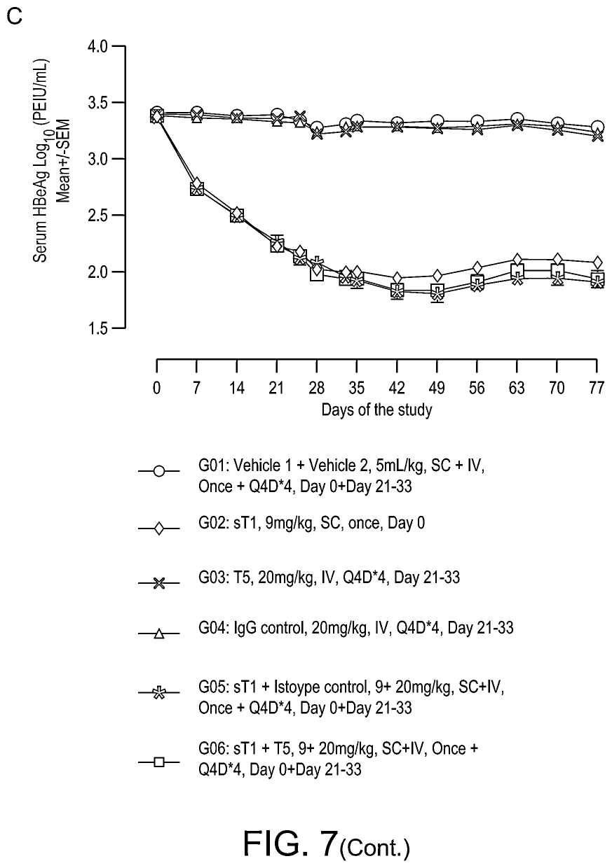

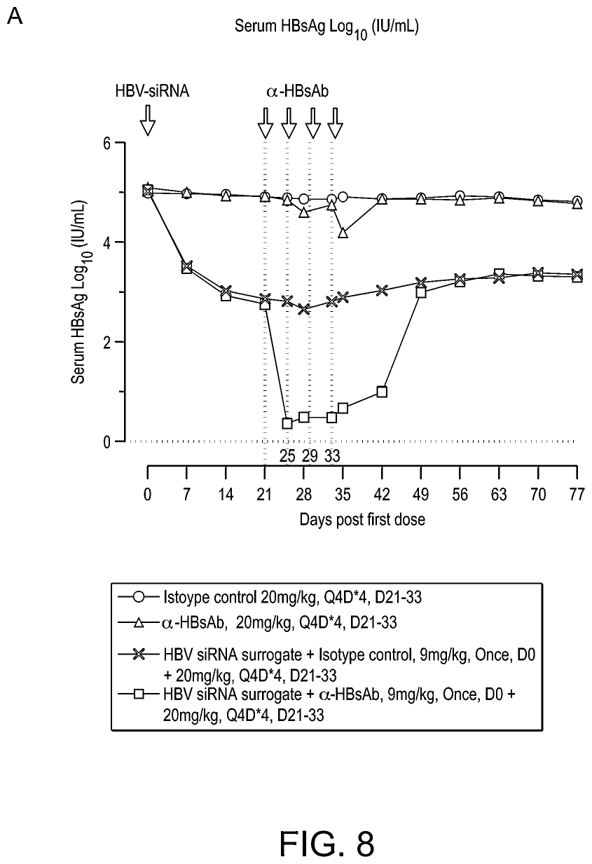

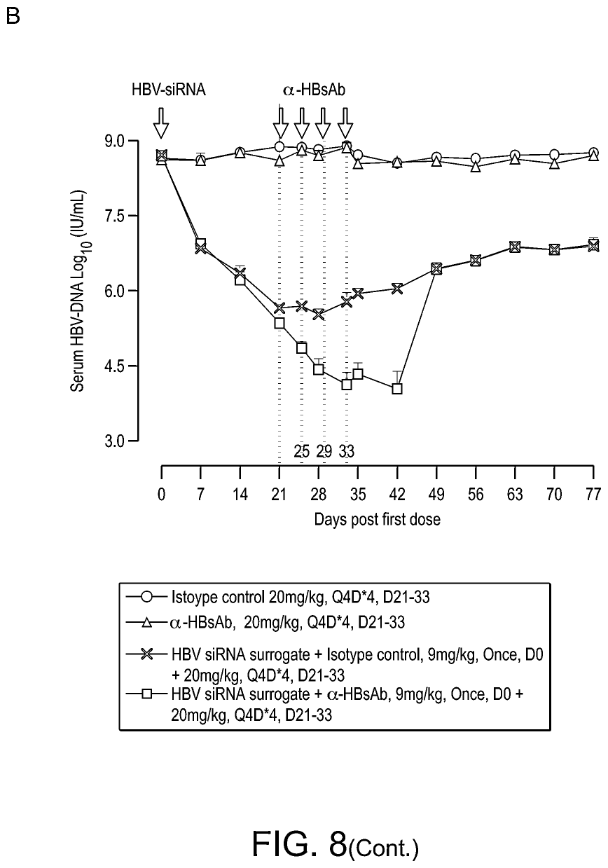

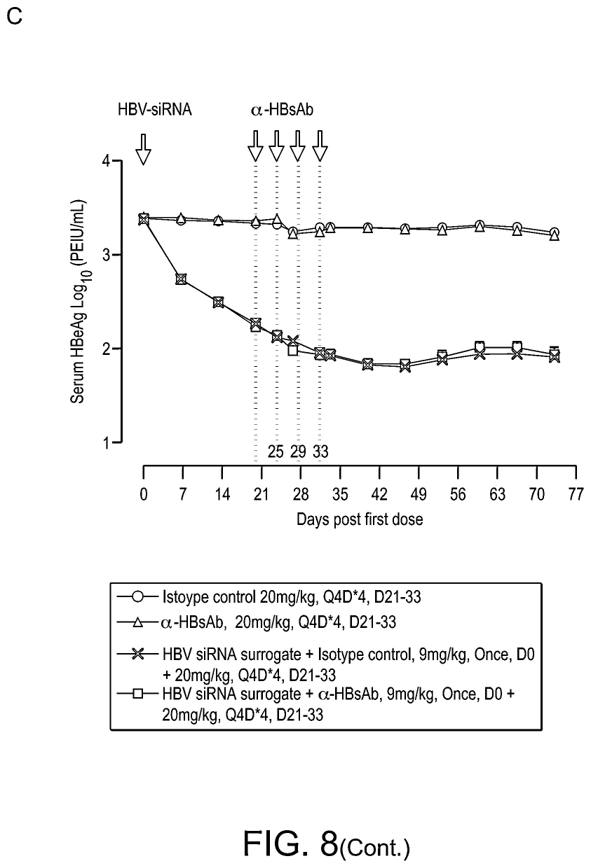

As shown in FIGS. 7 and 8, the serum levels of HBsAg, HBeAg and HBV-DNA in the vehicle control group maintained stable during Day 0-77. Compared to the group siRNA oligonucleotide targeting HBV (sT1) combined with the IgG control (Group 5), the combination with of a siRNA oligonucleotide targeting HBV (sT1) combined with an anti-HBV antibody (T5) (Group 06) significantly decreased serum levels of HBsAg and HBV-DNA during the anti-HBs antibody treatment period Day 21-33.

### Discussion

These results show the efficacy of pharmaceutical combinations comprising an RNAi oligonucleotide targeting HBV, such as T1 or sT1, and an anti-HBV antibody (anti-HBs), such as T5 used in this study, in vivo. The pharmaceutical combination comprising both an RNAi oligonucleotide targeting HBV and an anti-HBV antibody (Group 06) achieved rapid and significantly decreased levels of the HBV serum markers HBsAg and HBV-DNA, indicating a strong inhibitory effect on HBV. Now that these effects have been proven for the inventive pharmaceutical combination in vivo in mice (when utilizing the surrogate therapeutic sT1), it is to be understood that equivalent results can be obtained for the inventive pharmaceutical combination when using the equivalent therapeutics, such as T1, in vivo in other cells/tissues/subjects, such as that of a human.

1-2. (canceled)

3. A pharmaceutical combination for treating HBV, wherein the combination comprises an RNAi oligonucleotide targeting HBV and an anti-PDL1 antisense oligonucleotide, wherein the RNAi oligonucleotide is a siRNA oligonucleotide that targets HBsAg mRNA and reduces the expression of HBsAg mRNA.

4. (canceled)

5. The pharmaceutical combination of claim 3, wherein the RNAi oligonucleotide is an oligonucleotide comprising an antisense strand of 19 to 30 nucleotides in length, wherein the antisense strand comprises a region of complementarity to a sequence of HBsAg mRNA set forth as ACAANAAUCCUCACAAUA (SEQ ID NO: 1).

6. The pharmaceutical combination of claim 3, wherein the RNAi oligonucleotide comprises a sense strand that has a region of complementarity to the sequence set forth as UUNUUGUGAGGAUUN (SEQ ID NO: 2).

7. The pharmaceutical combination of claim 3, wherein the RNAi oligonucleotide comprises a sense strand comprising a sequence GACAANAAUCCUCACAAUAAGCAGCCGAAAGGCUGC (SEQ ID NO: 8) wherein:
(a) one or more of the nucleotides of the -GAAA- sequence on the sense strand are conjugated to a GalNac moiety; or
(b) wherein one or more of the nucleotides of the -GAAA- sequence on the sense strand are conjugated to a GalNac moiety and the RNAi oligonucleotide further comprises an antisense strain comprising a sequence UUAUUGUGAGGAUUNUUGUCGG (SEQ ID NO: 4).

8. The pharmaceutical combination of claim 3, wherein the RNAi oligonucleotide is an oligonucleotide comprising a sense strand forming a duplex region with an antisense strand, wherein:
(a) the sense strand consists of a sequence GACAAAAAUCCUCACAAUAAGCAGCCGAAAGGCUGC (SEQ ID NO: 9) and comprising 2′-fluoro modified nucleotides at positions 3, 8-10, 12, 13, and 17, 2′-O-methyl modified nucleotides at positions 1, 2, 4-7, 11, 14-16, 18-26, and 31-36, and a phosphorothioate linkage between the nucleotides at positions 1 and 2, wherein:
(i) each of the nucleotides of the -GAAA- sequence on the sense strand is conjugated to a monovalent GalNac moiety; or
(ii) each of the nucleotides of the -GAAA- sequence on the sense strand is conjugated to a monovalent GalNac moiety and the -GAAA- sequence comprises the structure: 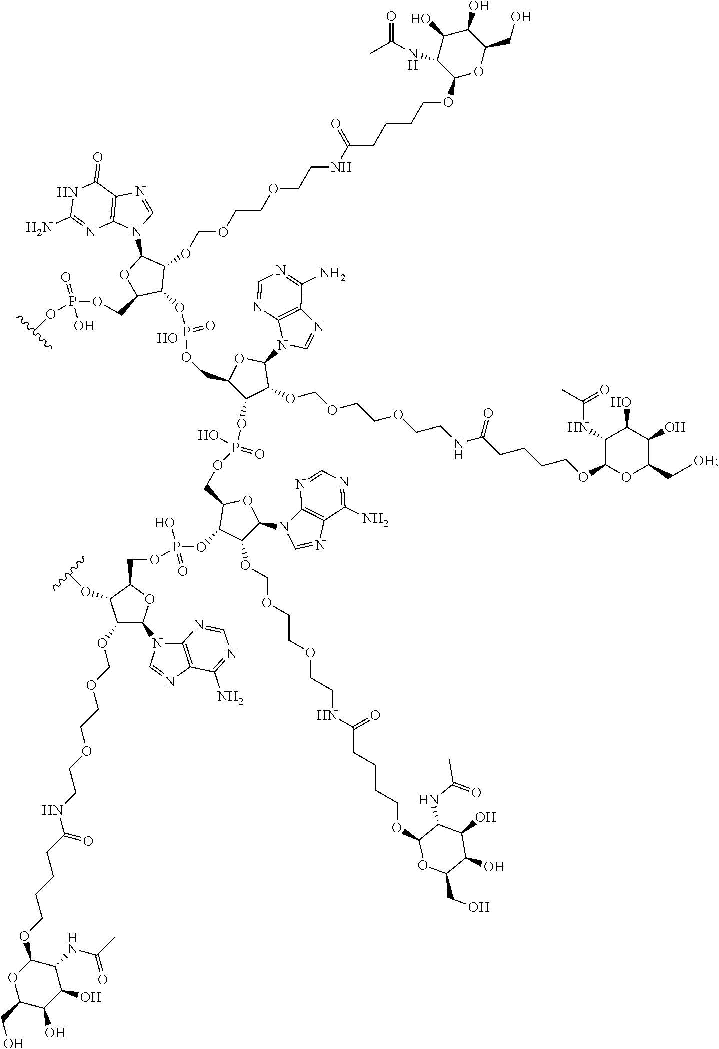 [SMILES: *O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(=O)[nH]c(N)nc32)O[C@@H]1COP(=O)(O)O*]
and
(b) the antisense strand consists of a sequence UUAUUGUGAGGAUUUUUGUCGG (SEQ ID NO: 6) and comprising 2′-fluoro modified nucleotides at positions 2, 3, 5, 7, 8, 10, 12, 14, 16, and 19, 2′-O-methyl modified nucleotides at positions 1, 4, 6, 9, 11, 13, 15, 17, 18, and 20-22, and phosphorothioate linkages between nucleotides at positions 1 and 2, between nucleotides at positions 2 and 3, between nucleotides at positions 3 and 4, between nucleotides at positions 20 and 21, and between nucleotides at positions 21 and 22, wherein:
(i) the 4′-carbon of the sugar of the 5′-nucleotide of the antisense strand comprises a methoxy phosphonate (MOP); or
(ii) the 4′-carbon of the sugar of the 5′-nucleotide of the antisense strand comprises a methoxy phosphonate (MOP) and the 5′-nucleotide of the antisense strand has the following structure: 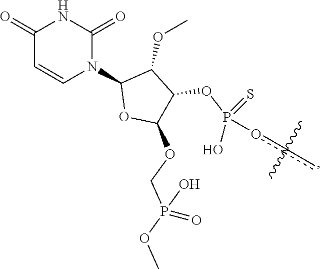
or a pharmaceutically acceptable salt thereof.

9-11. (canceled)

12. The pharmaceutical combination of claim 3, wherein:
(a) the anti-PDL1 antisense oligonucleotide comprises the sequence CCTATTTAACATCAGAC (SEQ ID NO: 11); or
(b) the anti-PDL1 antisense oligonucleotide comprises the sequence CCTATTTAACATCAGAC (SEQ ID NO: 11) and the anti-PDL1 antisense oligonucleotide has the formula GN2-C6ₒcₒaₒCCtatttaacatcAGAC, wherein C6 represents an amino alkyl group with 6 carbons, capital letters represent beta-D-oxy LNA nucleosides, lowercase letters represent DNA nucleosides, all LNA C are 5-methyl cytosine, subscript ₒ represents a phosphodiester nucleoside linkage, and unless otherwise indicated, all internucleoside linkages are phosphorothioate internucleoside linkages, and wherein GN2 represents the following trivalent GalNAc cluster: 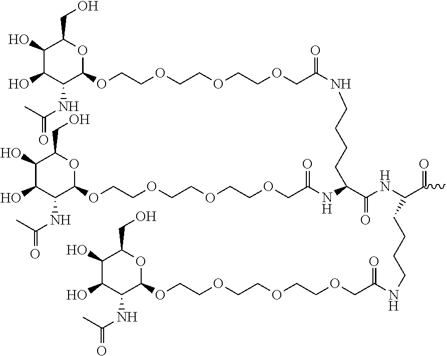
and further wherein the wavy line of the trivalent GalNAc cluster illustrates the site of conjugation of the trivalent GalNAc cluster to the C6 amino alkyl group;
or a pharmaceutically acceptable salt thereof.

13-15. (canceled)

16. The pharmaceutical combination of claim 3, wherein the combination is capable of reducing serum HBsAg, HBeAg, and/or HBV-DNA in a patient, wherein the reduction is greater than the sum of a) the reduction provided by the same dose of the RNAi oligonucleotide targeting HBV when administered without an anti-PDL1 antisense oligonucleotide, and b) the reduction provided by the same dose of the anti-PDL1 antisense oligonucleotide when administered without an RNAi oligonucleotide targeting HBV.

17. The pharmaceutical combination of claim 3, wherein:
(a) the RNAi oligonucleotide targeting HBV is present in an amount which will result in a dose of at least about 0.1 mg/kg to about 12 mg/kg, or a dose of at least about 0.5 mg/kg, or a dose of at least about 1 mg/kg, or a dose of at least about 1.5 mg/kg, or a dose of at least about 2 mg/kg, or a dose of at least about 3 mg/kg, or a dose of at least about 6 mg/kg, or a dose of at least about 9 mg/kg; and/or
(b) the anti-PDL1 antisense oligonucleotide is present in an amount which will result in a dose of at least about 0.1 mg/kg to about 35 mg/kg, or a dose of at least about 0.5 mg/kg, or a dose of at least about 1 mg/kg, or a dose of at least about 1.5 mg/kg, or a dose of at least about 2 mg/kg, or a dose of at least about 3 mg/kg, or a dose of at least about 6 mg/kg, or a dose of at least about 9 mg/kg.

18-24. (canceled)

25. The pharmaceutical combination of claim 3, further comprising:
(a) a further, different HBV therapeutic; or
(b) a TLR7 agonist, interferon-alpha, an anti-HBV antibody, an antibody which inhibits PD1 signalling, or a nucleotide analogue.

26-30. (canceled)

31. A composition comprising the pharmaceutical combination of claim 3.

32. A kit of parts comprising the RNAi oligonucleotide targeting HBV of claim 3, wherein the kit further comprises:
(A) instructions for administration of the RNAi oligonucleotide and the anti-PDL1 antisense oligonucleotide to treat a hepatitis B virus infection;
(B) instructions for subcutaneous administration of the RNAi oligonucleotide and subcutaneous administration of the anti-PDL1 antisense oligonucleotide to treat a hepatitis B virus infection; or
(C) instructions for administration of the RNAi oligonucleotide and the anti-PDL1 antisense oligonucleotide to treat a hepatitis B virus infection; and wherein:
(a) the RNAi oligonucleotide is an oligonucleotide comprising a sense strand forming a duplex region with an antisense strand, wherein:
(i) the sense strand consists of a sequence GACAAAAAUCCUCACAAUAAGCAGCCGAAAGGCUGC (SEQ ID NO: 9) and comprising 2′-fluoro modified nucleotides at positions 3, 8-10, 12, 13, and 17, 2′-O-methyl modified nucleotides at positions 1, 2, 4-7, 11, 14-16, 18-26, and 31-36, and a phosphorothioate linkage between the nucleotides at positions 1 and 2, wherein:
(1) each of the nucleotides of the -GAAA- sequence on the sense strand is conjugated to a monovalent GalNac moiety; or
(2) each of the nucleotides of the -GAAA- sequence on the sense strand is conjugated to a monovalent GalNac moiety and the -GAAA- sequence comprises the structure: 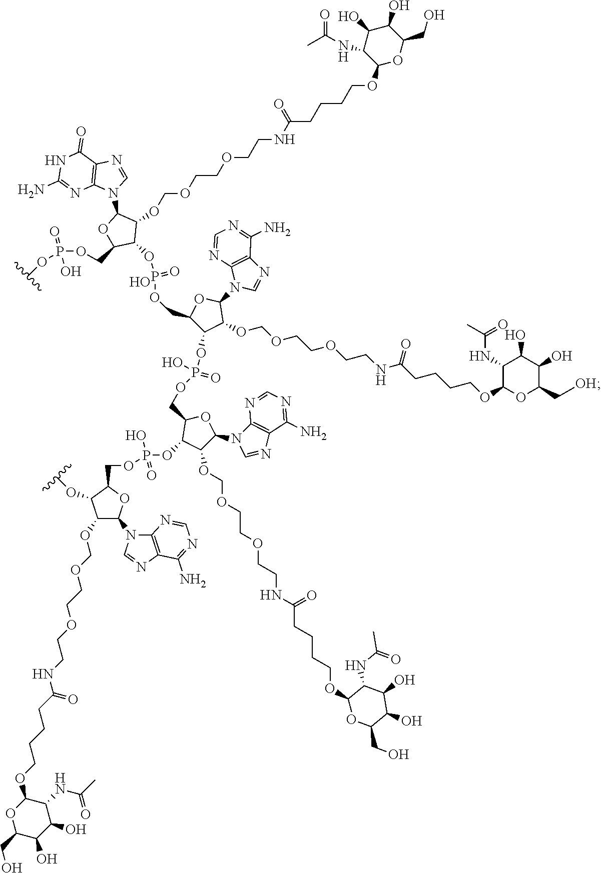 [SMILES: *O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(=O)[nH]c(N)nc32)O[C@@H]1COP(=O)(O)O*]
and
(ii) the antisense strand consists of a sequence UUAUUGUGAGGAUUUUUGUCGG (SEQ ID NO: 6) and comprising 2′-fluoro modified nucleotides at positions 2, 3, 5, 7, 8, 10, 12, 14, 16, and 19, 2′-O-methyl modified nucleotides at positions 1, 4, 6, 9, 11, 13, 15, 17, 18, and 20-22, and phosphorothioate linkages between nucleotides at positions 1 and 2, between nucleotides at positions 2 and 3, between nucleotides at positions 3 and 4, between nucleotides at positions 20 and 21, and between nucleotides at positions 21 and 22, wherein:
(1) the 4′-carbon of the sugar of the 5′-nucleotide of the antisense strand comprises a methoxy phosphonate (MOP); or
(2) the 4′-carbon of the sugar of the 5′-nucleotide of the antisense strand comprises a methoxy phosphonate (MOP) and the 5′-nucleotide of the antisense strand has the following structure: 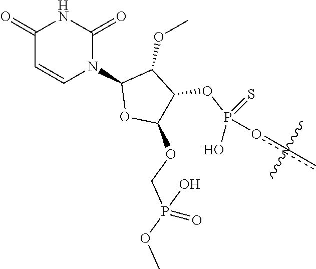
or a pharmaceutically acceptable salt thereof; and/or
(b) the anti-PDL1 antisense oligonucleotide comprises:
(i) the sequence CCTATTTAACATCAGAC (SEQ ID NO: 11); or
(ii) the sequence CCTATTTAACATCAGAC (SEQ ID NO: 11), and the anti-PDL1 antisense oligonucleotide has the formula GN2-C6ₒcₒaₒCCtatttaacatcAGAC, wherein C6 represents an amino alkyl group with 6 carbons, capital letters represent beta-D-oxy LNA nucleosides, lowercase letters represent DNA nucleosides, all LNA C are 5-methyl cytosine, subscript ₒ represents a phosphodiester nucleoside linkage, and unless otherwise indicated, all internucleoside linkages are phosphorothioate internucleoside linkages, and wherein GN2 represents the following trivalent GalNAc cluster: 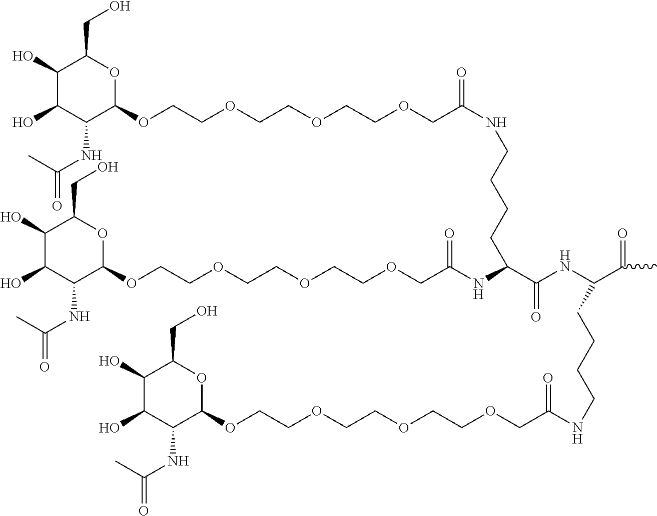
and further wherein the wavy line of the trivalent GalNAc cluster illustrates the site of conjugation of the trivalent GalNAc cluster to the C6 amino alkyl group;
or a pharmaceutically acceptable salt thereof.

33-36. (canceled)

37. The pharmaceutical combination, composition or kit of claim 3, wherein the RNAi oligonucleotide targeting HBV and/or the anti-PDL1 antisense oligonucleotide are in the form of a transgene engineered to express the oligonucleotide in a cell.

38-114. (canceled)

115. A method for treating a hepatitis B virus infection comprising administering a therapeutically effective amount of the pharmaceutical combination of claim 3 to a subject infected with a hepatitis B virus infection.

116. The method of claim 115, wherein:
(a) a single or initial dose of the RNAi oligonucleotide targeting HBV is administered prior to the administering of a single or initial dose of the anti-PDL1 antisense oligonucleotide; or
(b) a single or initial dose of the anti-PDL1 antisense oligonucleotide is administered at least about a week, at least about two weeks, at least about three weeks, at least about four weeks, at least about five weeks, at least about six weeks, at least about seven weeks, at least about eight weeks or longer than eight weeks after a single or initial dose of the RNAi oligonucleotide targeting HBV.

117-118. (canceled)

119. The method of claim 115, wherein the RNAi oligonucleotide targeting HBV is administered in weekly doses, and at least two doses are administered; and/or the anti-PDL1 antisense oligonucleotide is administered in weekly doses, and at least two doses or at least 5 doses are administered.

120-121. (canceled)

122. The method of claim 115, wherein the pharmaceutical combination is administered over the course of 48 weeks.

123. (canceled)

124. The method of claim 115, wherein:
(a) the RNAi oligonucleotide targeting HBV is (i) in a dosage form for subcutaneous administration and/or administered in a dose of at least about 0.1 mg/kg, or a dose of at least about 0.5 mg/kg, or a dose of at least about 1 mg/kg, or a dose of at least about 1.5 mg/kg, or a dose of at least about 2 mg/kg, or a dose of at least about 3 mg/kg, or a dose of at least about 6 mg/kg, or a dose of at least about 9 mg/kg; and/or
(b) the anti-PDL1 antisense oligonucleotide is (i) in a dosage form for subcutaneous administration; (ii) administered in up to give doses; (iii) administered at least every two weeks; and/or (iv) administered in a dose of at least about 0.1 mg/kg to about 35 mg/kg, or a dose of at least about 0.5 mg/kg, or a dose of at least about 1 mg/kg, or a dose of at least about 1.5 mg/kg, or a dose of at least about 2 mg/kg, or a dose of at least about 3 mg/kg, or a dose of at least about 6 mg/kg, or a dose of at least about 7 mg/kg, or a dose of about 7 mg/kg to about 35 mg/kg.

125-129. (canceled)

130. The method of claim 115, wherein two, five, or more doses of anti-PDL1 antisense oligonucleotide are administered, once weekly, with the initial dose of anti-PDL1 antisense oligonucleotide being administered at least about 7 days after the dose of the RNAi oligonucleotide targeting HBV; and the doses of the anti-PDL1 antisense oligonucleotide are at least about 3 mg/kg.

131-138. (canceled)

139. The method of claim 115, wherein the RNAi oligonucleotide targeting HBV and/or the anti-PDL1 antisense oligonucleotide are delivered in the form of a transgene that is engineered to express the oligonucleotide in a cell.

140-145. (canceled)
